(19) United States
(12) Patent Application Publication
(10) Pub. No.: US 2010/0048604 A1
(43) Pub. Date: Feb. 25, 2010

(54) Ligands for Aldoketoreductases

(76) Inventors: Dominic J. Yee, San Francisco, CA (US); Vojtech Balsanek, Trhovy Stepanov (CZ); Dalibor Sames, New York, NY (US)

Correspondence Address: COOPER & DUNHAM, LLP, 30 Rockefeller Plaza, 20th Floor, NEW YORK, NY, 10112, US

(21) Appl. No.: 11/660,687

(22) PCT Filed: Aug. 19, 2005
(86) PCT No.: PCT/US05/29722

§ 371 Date: Aug. 13, 2007

Related U.S. Application Data
(60) Provisional application No. 60/603,311, filed on Aug. 20, 2004.

(51) Int. Cl. A61K 31/4375; C07D 471/16; C07C 211/45; C07D 311/02; A61K 31/136; A61K 31/352; C12Q 1/68

(52) U.S. Cl. 514/285; 546/62; 564/429; 549/399; 514/657; 514/457; 435/6

(57) ABSTRACT

The present invention relates to compounds useful for detecting the activity of human aldoketoreductase 1Cs, compounds useful for competitively inhibiting human aldoketoreductase 1Cs and compounds useful for treating human aldoketoreductase 1C-related cancers, as well as pharmaceutical compositions and methods of manufacture thereof.

This application is a continuation-in-part and claims priority of U.S. Provisional Application No. 60/603,311, filed Aug. 20, 2004, the contents of which are hereby incorporated by reference.

Throughout this application, various publications are referenced by complete citation in parentheses. The disclosures of these publications in their entireties are hereby incorporated by reference into this application in order to more fully describe the state of the art as known to those skilled therein as of the date of the invention described and claimed herein.

### BACKGROUND OF THE INVENTION

Molecular imaging of metabolic and signaling events in living systems represents an important frontier in life sciences and medicine. The ability to observe functioning cells, tissues, and organs with high levels of molecular and dynamic resolution will propel a wide spectrum of human activities, including scientific, philosophical, and medicinal fields (Weissleder, R. and Ntziachristos, V. Nature Med. 2003, 9, 123-128). One promising approach for non-invasive metabolic measurements stands on the use of small molecule reporters, such as fluorogenic probes which provide a measurable optical signal for a particular enzyme facilitated molecular process ((a) Moreira R., Havranek M., Sames D. J. Am. Chem. Soc. 2001, 123, 3927-3931. (b) Chen, C.-A.; Yeh, R.-H.; Lawrence, D. S. J. Am. Chem. Soc. 2002, 124, 3840-3841).

Many fluorogenic probes consist of an organic dye attached at the periphery of a natural substrate wherein the emission change is usually achieved via fluorescence energy transfer (FRET) (Boonacker E., Van Noorden C. J. F.: J. Histochem. Cytochem. 2001, 49, 1473-1486. (b) Handbook of Fluorescent Probes and Research Chemicals, Molecular Probes, 9^{th} ed.; Haugland, R. P., Ed.; 2002) or a phenol- or aniline-releasing reaction ((a) Wang, G. T.; Matayoshi, E.; Huffaker, H. J.; Krafft, G. A. Tetrahedron Lett. 1990, 31, 6493-6496; (b) Rotman, B.; Zderic, J. A.; Edelstein, M. Proc. Natl. Acad. Sci. USA 1963, 50, 1-6. (c) Zimmerman, M,; Ashe, B.; Yurewicz, E.; Patel, G. Anal. Biochem. 1977, 78, 47-51). For instance, a short peptide equipped with an appropriate dye attached at the N-terminus illustrates a common design for protease probes Alcohol dehydrogenase probes which require two catalytic steps (oxidation and β-elimination), (Klein, G.; Reymond, J.-L. Bioorg. Med. Chem. Lett. 1998, 8, 1113-1116). In these cases the enzyme recognizes the natural substrate while the organic dye resides outside the enzyme's perimeter, thereby minimizing reporter-enzyme interactions (Rettig, W. Angew. Chem. Int. Ed. 1986, 25, 971-988). However, in cases where these mechanisms are not applicable, the organic dye may become an integral part of the recognized substrate. In this latter instance, a synthetic molecule, bearing minimal resemblance to a physiological substrate, would have to function as a competitive substrate (previous examples of carbonyl-alcohol fluorogenic probes suffered from short excitation/emission wavelengths in the near UV region. (a) Wierzchowski, J.; Dafeldecker, W. P.; Holmquist, B.; Vallee, B. L. Anal. Biochem. 1989, 178, 57-62. (b) List, B.; Barbas III, C. F.; Lerner, R. A. Proc. Natl. Acad. Sci. USA 1998, 95, 15351-15355).

The enzymes of interest discussed here, oxidoreductases, including alcohol dehydrogenases, play essential roles in maintaining the balance of metabolic energy and regulating the concentration of critical metabolites, hormones, and xenobiotics. Redox optical probes must have a built-in mechanism for coupling the chemical redox event to a switch in emission properties. However, two mechanisms frequently used for construction of fluorogenic substrates (e.g. probes for hydrolases), namely fluorescence energy transfer (FRET) and phenol- or anilin-releasing reactions are generally not suitable for alcohol dehydrogenase probes.

Hydroxysteroid dehydrogenases (HSDs) that belong to the aldo-keto reductase superfamily (AKR) (Fang, J.-M.; Lin, C.-H.; Bradshaw, C. W.; Wong, C.-H. J. Chem. Soc. Perkin Trans. 1 1995, 967-978) may play important roles in steroid hormone action. There are four known human isozymes, designated as AKR1C1, AKR1C2, AKR1C3, and AKR1C4, which exhibit different expression levels in various tissues (Penning, T. M.; Burczynski, M. E.; Jez, J. M.; Hung, C.-F.; Lin, H.-K.; Ma, H.; Moore, M.; Palackal, N.; Ratnam, K. Biochem. J. 2000, 351, 67-77). It has been proposed that these HSDs function as pre-receptor switches by activating/deactivating steroid hormones via redox chemistry. For example, the occupancy of androgen receptors in the prostate may be regulated by reducing the highly potent androgen 5α-dihydrotestosterone to the inactive metabolite 3α-androstanediol. Similarly, reduction of 5α-dihydroprogesterone to 3α,5α-tetrahydroprogesterone (allopregnanolone) produces an allosteric regulator of the GABA receptor in the brain. Both reactions are catalyzed by human type 3 3α-hydroxysteroid dehydrogenase (AKR1C2). By contrast, AKR1C3 contains high 17β-HSD activity and it is involved in the peripheral formation of androgens and estrogens, reactions that may be important in prostate and breast cancer. Moreover, AKR1C3 also exhibits prostaglandin synthase activity.

AKR1C2 and AKR1C3 are of particular interest. In fact, AKR1C2 levels are elevated in epithelial cells from prostate cancer; and this may contribute to the development of androgen independent tumors. These findings together with the physiological functions of HSDs provide a strong impetus for the development of selective imaging probes for these enzymes as well as competitive inhibitors of the enzymes.

The structure-function relationship of 3α-hydroxysteroid dehydrogenases has been studied in both rat and human isoforms (e.g. see Penning et al., J. Steroid Biochem. and Mol. Biol. 85, 247-255 (2003)). Furthermore, AKR1C3 has been identified as a suppressor of cell differentiation in myeloid cells, and has been suggested as an antineoplastic target (e.g. in HL-60 cells, see Desmond et al. Cancer Res. 63, 505-512, (2003)). Overexpression of AKR1C3 resulted in diminished sensitivity to the differentiation promoter ATRA. Inhibition of the activity of the enzyme, such as by competitive inhibition, could therefore be a useful cancer therapy. The capacity of NSAIDs to protect against certain tumors has been suggested to be due to the influence of NSAIDs on inhibition of AKR1C3 coupled with the wide tissue distribution of the enzyme. In addition, gut (e.g. colon) and prostate cancers share a common etiology and diets high in vegetable content can offer protection. It has been suggested that such protection may arise from dietary plant constituents shown to inhibit AKR1C3 (see Desmond et al. 2003).

More generally, Hsu et al. (Cancer Research 61, 2727-2731, 2001), using mRNA differential display, have demonstrated that overexpression of dihydriol dehydrogenase (DDH) (an AKR 1C) can be used as a prognostic marker of human non-small cell lung cancer, and that DDH overexpression was correlated with tumor recurrence, metastasis and patient survival.

Here, in the context of aldoketo-reductases, design, chemical synthesis, enzymatic screening, identification of leads, and development of new fluorogenic probes for 3a-hydroxysteroid dehydrogenases (AKR1Cs) are disclosed, as well as competitive inhibitors of the AKR1Cs and nonphysiological substrates.

### SUMMARY OF THE INVENTION

One embodiment of this invention provides a compound of the structure:

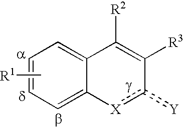

wherein
   Y is O, X is O, and bond γ is a single bond, or
   Y is absent, X is CH and bond γ is a double bond,

wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl—C(O)H, -aryl-CH₂OH, -aryl-C(O)OH, -alkynyl-C(O)H, -alkynyl-CH₂OH, or -alkynyl-C(O)OH; or R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,

or R¹ is bound to carbon α and is —N(alkyl)₂, R² is —C(O)H, —CH₂OH, —C(O)OH, —C(O)CH₃, —CH(OH)CH₃, and R³ is H, or R¹ is bound to carbon α and is —O-alkyl, R² is —CH(OH)CH₃ or —C(O)OH, and R³ is H,

or R¹ is bound to carbon β and is —O-alkyl, R² is —C(O)H, —C(O)OH, —CH₂OH, —C(O)CH₃, —CH(OH)CH₃, and R³ is H,

or R¹ is bound to carbon β and is —N(alkyl)₂, R² is —C(O)H, —C(O)OH, —CH₂OH, —C(O)CH₃, —CH(OH)CH₃, and R³ is H,

or R¹ is bound to carbon 8 and is —N< which is covalently bound to both carbon α and carbon β and either R² is —H and R³ is —C(O)H, —CH₂OH, -aryl—C(O)H, -aryl—CH₂OH, -aryl-C(O)OH, -alkynyl—C(O)H, -alkynyl—CH₂OH, —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where —X is a halide, —C(CHX)(aryl) where X is a halide, —C(═NOH)(aryl), —CH(CH₃)(aryl), —CH₂-(aryl), or —C(CH₂)(aryl); or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl,

wherein when R¹ is —N(CH₃)₂ and is bound at carbon δ and R³ is —C(O)CH₃ or —CH(OH)(CH₃), or R¹ is —O-alkyl and is bound at carbon 5 and R³ is —C(O)H, then R² is OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴, Y is O, X is O and bond γ is a single bond,

or a salt or stereoisomer thereof.

### BRIEF DESCRIPTION OF THE FIGURES

FIG. 1. Design of an optical switch based on carbonyl-alcohol redox chemistry. EDG=Electron-Donating Group, EWG=Electron-Withdrawing Group.

FIG. 2. Synthesis of compound arrays based on three fluorophore cores (the corresponding alcohols are not shown).

FIG. 3. Selected probes 1-7.

FIG. 4. Screening of probes 1-7 against a panel of oxidoreductases Percentage fluorescence increase after 12 hour incubation of 30-50 μM substrate, 100 mM phosphate buffer (pH 7), 250 μM NAD(P)H, and 100 nM enzyme. Substrates 1, 2, 3, and 4 monitored at λ_{exc}=340 nm, λₑₘ=440 nm. Substrates 5, 6, and 7 monitored at λ_{exc}=440 nm, λₑₘ=510 nm. 3HSD, 3α-hydroxysteroid dehydrogenase (PT, Pseudomonas testosteroni), HLAD, horse liver alcohol dehydrogenase, TBAD, Thermoanaerobium brockii alcohol dehydrogenase, BS 12HSD, Bacillus sp. 12α-hydroxysteroid dehydrogenase, ABAD, amyloid-β binding alcohol dehydrogenase (human), GDH, glycerol dehydrogenase, YADH, yeast alcohol dehydrogenase, LDH, lactate dehydrogenase.

FIG. 5. Kinetic parameters for probe 5 and the physiological substrate for human 3α-HSD (type 2, AKR1C3).

FIG. 6. Enzyme kinetic data for AKR1C3.

FIG. 7. Fluorescence spectra for probe 1 (trace A is alcohol, trace B is ketone).

FIG. 8. Fluorescence spectra for probe 2 (trace A is alcohol, trace B is ketone).

FIG. 9. Fluorescence spectra for probe 3 (trace A is alcohol, trace B is ketone).

FIG. 10. Fluorescence spectra for probe 4 (trace A is alcohol, trace B is ketone).

FIG. 11. Fluorescence spectra for probe 5 (trace A is alcohol, trace B is ketone).

FIG. 12. Fluorescence spectra for probe 6 (trace A is alcohol, trace B is ketone).

FIG. 13. Fluorescence spectra for probe 7 (trace A is alcohol, trace B is ketone).

FIG. 14. Proposed physiological roles for hydroxysteroid dehydrogenases.

FIG. 15. Probe 5-derived fluorogenic substrates.

FIG. 16. Graphical representation of the selectivity profile of the probe 5-derived active fluorogenic substrates against four known human 3α-HSD isozymes. Legend: (a) AKR1C1; (b) AKR1C2; (c) AKR1C3; (d) AKR1C4.

FIG. 17. Reactivity of HepG2 cell fractions with 5c. Assays were performed in 50 mM Tris-HCl buffer containing 1 mM NADPH, 10 μM 5c and 80 μg protein/mL, incubated for 60 minutes. Legend: (+) without flufenamic acid; (−) with 100 μM flufenamic acid; HepG2=hepatoma cell line.

### DETAILED DESCRIPTION

This invention provides a compound of the structure:

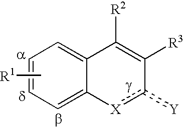

wherein
   Y is O, X is O, and bond γ is a single bond, or
   Y is absent, X is CH and bond γ is a double bond,

wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl—CH₂OH, -aryl-C(O)OH, -alkynyl—C(O)H, -alkynyl—CH₂OH, or -alkynyl-C(O)OH; or R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,

or R¹ is bound to carbon α and is —N(alkyl)₂, R² is —C(O)H, —CH₂OH, —C(O)OH, —C(O)CH₃, —CH(OH)CH₃, and R³ is H,

or R¹ is bound to carbon α and is —O-alkyl, R² is —CH(OH)CH₃ or —C(O)OH, and R³ is H,

or R¹ is bound to carbon β and is —O-alkyl, R² is —C(O)H, —C(O)OH, —CH₂OH, —C(O)CH₃, —CH(OH)CH₃, and R³ is H,

or R¹ is bound to carbon β and is —N(alkyl)₂, R² is —C(O)H, —C(O)OH, —CH₂OH, —C(O)CH₃, —CH(OH)CH₃, and R³ is H,

or R¹ is bound to carbon β and is —N< which is covalently bound to both carbon α and carbon β and either R² is —H and R³ is —C(O)H, —CH₂OH, -aryl—C(O)H, -aryl—CH₂OH, -aryl-C(O)OH, -alkynyl—C(O)H, -alkynyl—CH₂OH, —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where —X is a halide, —C(CHX)(aryl) where X is a halide, —C(═NOH)(aryl), —CH(CH₃)(aryl), —CH₂-(aryl), or —C(CH₂)(aryl); or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl) , —CH(CH₃)(aryl), —CH₂-(aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl,

wherein when R¹ is —N(CH₃)₂ and is bound at carbon δ and R³ is —C(O)CH₃ or —CH(OH)(CH₃), or R¹ is —O-alkyl and is bound at carbon δ and R³ is —C(O)H, then R² is OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴, Y is O, X is O and bond γ is a single bond,

or a salt or stereoisomer thereof.

This invention provides the instant compound wherein when R¹ is —O—CH₃ and bound at carbon δ and R³ is —C(O)H, —C(O)CH₃ or —CH(OH)(CH₃), Y is absent, X is CH and bond γ is a double bond, then R² is OH, a C₂-C7 alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴, where R^{4,} is ethyl, alkenyl, alkynyl, substituted aryl or unsubstituted aryl.

This invention provides the instant compound wherein when R¹ is bound to carbon α and is —O-alkyl, R² is —CH(OH)CH₃ or —C(O)OH, and R³ is H, then Y is O, X is O, and bond γ is a single bond. This invention provides the instant compound wherein when R¹ is bound to carbon β and is —O-alkyl, R² is —C(O)H, —CH₂OH, —C(O)CH₃ or C(O)OH, and R³ is H, then Y is O, X is O, and bond γ is a single bond. This invention provides the instant compound wherein when R¹ is bound to carbon β and is —N(alkyl)₂, R² is —C(O)H, or —CH₂OH, and R³ is H, then Y is O, X is O, and bond γ is a single bond. This invention provides the instant compound wherein when R¹ is bound to carbon δ and is H, R² is —H or —O—CH₃, and R³ is —C(O)H, then Y is O, X is O, and bond γ is a single bond. This invention provides the instant compound wherein R¹ is bound at carbon δ and is >NH which is covalently bound to carbon α or to carbon β and the nitrogen atom and/or a carbon atom is substituted with one or more of an alkyl, alkylene-X where X is a halide, alkylene-C(O)OH, alkenyl, alkynyl, alkoxy, or alcohol.

This invention provides the instant compound, having the structure:

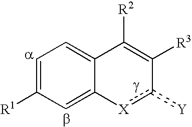

wherein
Y is O, X is O, and bond γ is a single bond, or
Y is absent, X is CH and bond γ is a double bond,

wherein R¹ is —H, —OH, —O-alkyl, —NH-alkyl, —N(alkyl)₂, —NH2, aryl, heteroaryl, -alkyl—C(O)(OH), -alkyl-OH, or R¹ is >NH which is covalently bound to carbon α or to carbon β; R² is H, OH, a C₂-C₇ alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, or —R⁵—CH(OH)R⁴; or R² and R³ together form a ring substituted with ═O,
where R⁴ is methyl, alkenyl, alkynyl, or aryl; R⁵ is alkyl, alkenyl, alkynyl, aryl, or cycloalkyl; and R⁶ is alkenyl, alkynyl, aryl, or cycloalkyl,

or R¹ is —N< which is covalently bound to both carbon a and carbon β and either R² is —H and R³ is —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where X is a halide, —C(CHX)(aryl) where X is a halide, —C(═NOH)(aryl), —CH(CH₃)(aryl), —CH₂-(aryl), or —C(CH₂)(aryl); or R³ is —H and R² is —C(O)R¹¹, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl); or R² and R³ together form a ring substituted with ═O,
where R⁷ is cycloalkyl, C₂-C₇ alkyl, alkenyl, alkynyl, aryl, or heteroaryl, R⁸ is hydroxyl, alkyl, cycloalkyl, alkenyl, alkynyl, aryl, or heteroaryl, R⁹ is alkyl, cycloalkyl, alkenyl, alkynyl, aryl, or heteroaryl, R¹⁰ is alkynyl, aryl, or heteroaryl, and R¹¹ is methyl, hydroxyl, alkenyl, alkynyl, aryl, or heteroaryl,

wherein when R¹ is —N(CH₃)₂ and R³ is —C(O)CH₃ or —CH(OH)(CH₃), then R² is OH, a C₂-C₇ alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁴, —CH(OH)R⁴, —R⁵—C(O)R⁴, or —R⁵—CH(OH)R4, Y is O, X is O and bond γ is a single bond,

or a salt or stereoisomer thereof.

This invention provides the instant compound wherein R¹ is —H, —OH, —O-alkyl, —NH-alkyl, —N(alkyl)₂, —NH₂, aryl, heteroaryl, -alkyl—C(O)(OH), -alkyl-OH, or R¹ is >NH which is covalently bound to carbon α or to carbon β; R² is —C(O)R⁴, —CH(OH)R⁴, —R⁵—C(O)R⁴, or —R⁵—CH(OH)R⁴; and R³ is —C(O)R⁶, —CH(OH)R⁴, —R⁵—C(O)R⁴, or —R⁵—CH(OH)R⁴; or R² and R³ together form a ring substituted with ═O,
where R⁴ is methyl, alkenyl, alkynyl, or aryl, R⁵ is alkyl, alkenyl, alkynyl, aryl, or cycloalkyl, and R⁶ is alkenyl, alkynyl, aryl, or cycloalkyl,

or R¹ is —N< which is covalently bound to both carbon a and carbon β and either R² is —H and R³ is —C(O)R⁷, —CH(OH)R⁸, —R¹⁰—C(O)R⁹, or —R¹⁰—CH(OH)R⁹; or R³ is —H and R² is —C(O)R¹¹, —CH(OH)R⁷, —R¹⁰—C(O)R⁹, or —R¹⁰—CH(OH)R⁹,

where R⁷ is cycloalkyl, C₂-C₇ alkyl, alkenyl, alkynyl, aryl, or heteroaryl; R⁸ is hydroxy, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, alkenyl, alkynyl, aryl, or heteroaryl.

This invention provides the instant compound wherein R¹ is —H, —OH, —O-alkyl, —NH-alkyl, —N(alkyl)₂, —NH₂, aryl, heteroaryl, -alkyl—C(O)(OH), -alkyl-OH, or R¹ is >NH which is covalently bound to carbon α or to carbon β; R² is H, OH, a C₂-C₇ alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide,

or R¹ is —N< which is covalently bound to both carbon a and carbon β and either R² is —H and R³ is —C(CX₂)(aryl) where X is a halide, —C(CX₂)(alkyl) where X is a halide, —C(CHX)(aryl) where X is a halide, —C(═NOH)(aryl), —CH(CH₃)(aryl), —CH₂-(aryl), or —C(CH₂)(aryl); or R³ is —H and R² is —C(CX₂)(aryl) where X is a halide, —C(CHX)(aryl) where X is a halide, —C(═NOH)(aryl), —CH(CH₃)(aryl), —CH₂-(aryl), —C(CH₂)(aryl), or —C(O)R¹¹ where R¹¹ is hydroxy.

This invention provides the instant compound having the structure:

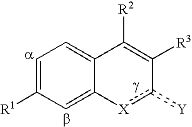

wherein Y is absent, X is CH, and γ is a double bond.

This invention provides the instant compound wherein R² is —C(O)R⁴, —CH(OH)R⁴, —R⁵—C(O)R⁴, or —R⁵—CH(OH)R⁴, and R³ is —C(O)R⁶, —CH(OH)R⁴, —R⁵—C(O)R⁴, or —R⁵—CH(OH)R⁴; or R² and R³ together form a ring substituted with ═O,
where R⁴ is methyl, alkenyl, alkynyl, or aryl; R⁵ is alkyl, alkenyl, alkynyl, aryl, or cycloalkyl; and R⁶ is alkenyl, alkynyl, aryl, or cycloalkyl.

This invention provides the instant compound, having the structure:

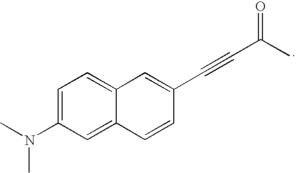

This invention provides the instant compound wherein R¹ is —H, —OH, —O-alkyl, —NH-alkyl, —N(alkyl)₂, —NH₂, aryl, heteroaryl, -alkyl—C(O)(OH), -alkyl—OH, or R¹ is >NH which is covalently bound to carbon α or to carbon β; R² is H, OH, a C₂-C₇ alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide; and R³ is —H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, or halide.

This invention provides the instant compound having the structure:

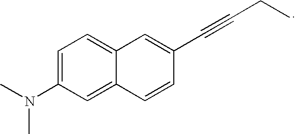

This invention provides the instant compound having the structure:

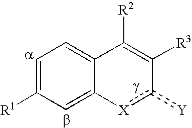

wherein X is O, Y is O, and γ is a single bond,
wherein R¹ is —H, —OH, —O-alkyl, —NH-alkyl, —N(alkyl)₂, —NH₂, aryl, heteroaryl, -alkyl—C(O)(OH), —alkyl—OH, or R¹ is >NH which is covalently bound to carbon α or to carbon β.

This invention provides the instant compound, wherein R² is —C(O)R⁴, —CH(OH)R⁴, —R⁵—C(O)R⁴, or —R⁵—CH(OH)R⁴, and R³ is —C(O)R⁶, —CH(OH)R⁴, —R⁵—C(O)R⁴, or —R⁵—CH(OH)R⁴; or R² and R³ together form a ring substituted with ═O,
where R⁴ is methyl, alkenyl, alkynyl, or aryl; R⁵ is alkyl, alkenyl, alkynyl, aryl, or cycloalkyl; and R⁶ is alkenyl, alkynyl, aryl, or cycloalkyl.

This invention provides the instant compound, having the structure:

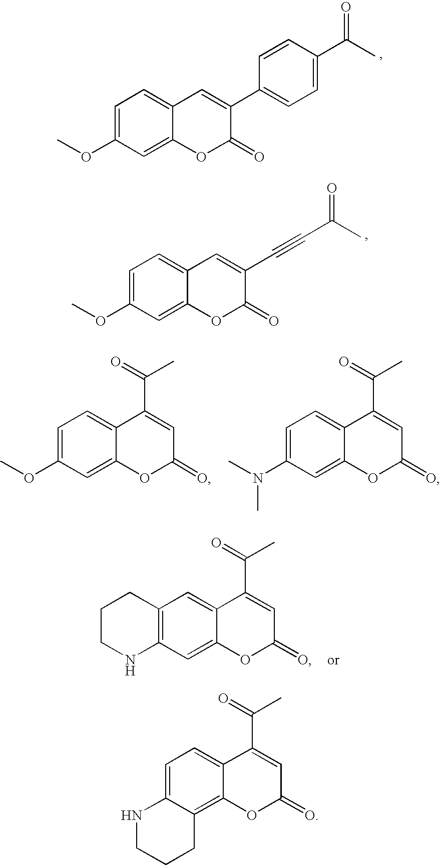

This invention provides the instant compound wherein R¹ is —H, —OH, —O-alkyl, —NH-alkyl, —N(alkyl)₂, —NH₂, aryl, heteroaryl, -alkyl—C(O)(OH), -alkyl—OH, or R¹ is >NH which is covalently bound to carbon α or to carbon β; R² is H, OH, a C₂-C₇ alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide; R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, or halide.

This invention provides the instant compound having the structure:

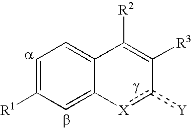

wherein X is O, Y is O, and γ is a single bond,

wherein R¹ is —N< which is covalently bound to both carbon α and carbon β.

This invention provides the instant compound, where R² is —H, and R³ is —C(O)R⁷, —CH(OH)R⁸, —R¹⁰—C(O)R⁹, or —R¹⁰—CH(OH)R⁹, and R³ is —H, and R² is —C(O)R¹¹, —CH(OH)R⁷, —R¹⁰—C(O)R⁹, —R¹⁰—CH(OH)R⁹; or R² and R³ together form a ring substituted with ═O,

where R⁷ is cycloalkyl, C₂-C₇ alkyl, alkenyl, alkynyl, aryl, or heteroaryl; R⁸ is hydroxy, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, alkenyl, alkynyl, aryl, or heteroaryl.

This invention provides the instant compound, having the structure:

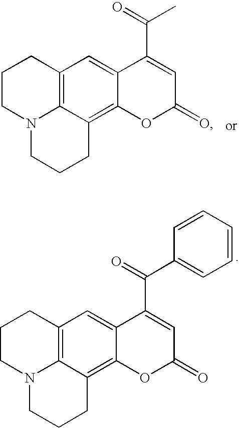

This invention provides the instant compound having the structure:

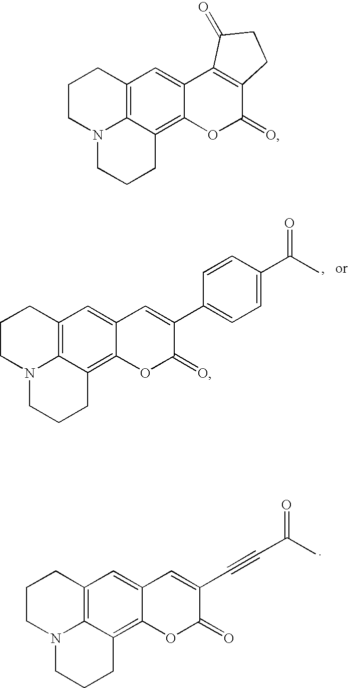

This invention provides the instant compound, where either R² is —H and R³ is —C(CX₂)(aryl) where X is a halide, —C(CHX)(aryl) where X is a halide, —C(CX₂)(alkyl) where X is a halide, —C(═NOH)(aryl), —CH(CH₃)(aryl), —CH₂-(aryl), or —C(CH₂)(aryl); or R³ is —H and R² is —C(CX₂)(aryl) where X is a halide, —C(CHX)(aryl) where X is a halide, —C(═NOH)(aryl), —CH(CH₃)(aryl), —CH₂-(aryl), —C(CH₂)(aryl), or —C(O)R¹¹ where R¹¹ is hydroxy.

This invention provides the instant compound, having the structure:

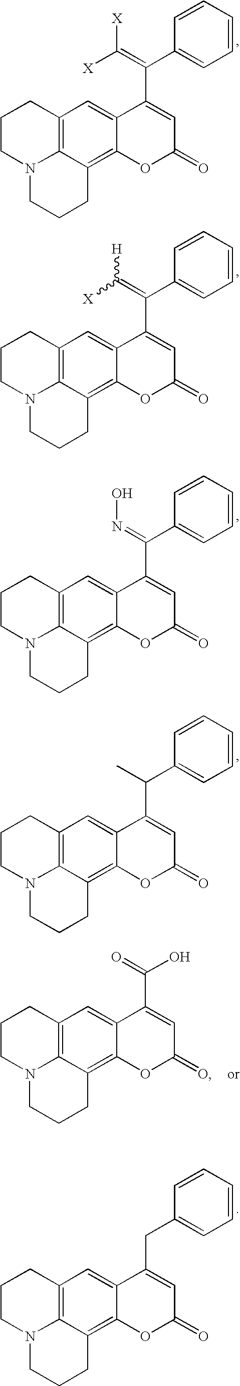

This invention provides the instant compound having the structure:

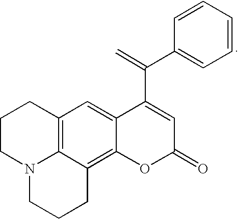

This invention provides the a compound of the structure:

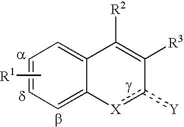

wherein
Y is O, X is O, and bond γ is a single bond, or
Y is absent, X is CH and bond γ is a double bond,

wherein R¹ is bound at carbon β and is —H, —OH, —O-alkyl, —NH-alkyl, —N(alkyl)₂, —NH₂, aryl, heteroaryl, -alkyl-C(O)(OH), -alkyl—OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl—CH₂OH, -aryl-C(O)OH, -alkynyl—C(O)H, -alkynyl—C(O)OH, or -alkynyl-CH₂OH; or R² and R³ together form a ring substituted with ═O,
where R⁴ is methyl, ethyl, alkenyl, alkynyl, a substituted aryl or an unsubstituted aryl, R⁵ is alkyl, alkenyl, alkynyl, substituted aryl or an unsubstituted aryl, or cycloalkyl, and R⁶ is hydrogen, methyl, a C₃-C₇ alkyl, alkenyl, alkynyl, aryl, or cycloalkyl,

or R¹ is bound to carbon α and is —N(alkyl)₂, R² is —C(O)H, —CH₂OH, —C(O)OH, —C(O)CH₃, —CH(OH)CH₃, and R³ is H, or R¹ is bound to carbon α and is —O-alkyl, R² is —CH(OH)CH₃ or —C(O)OH, and R³ is H,

or R¹ is bound to carbon β and is —O-alkyl, R² is —C(O)H, —C(O)OH, —CH₂OH, —C(O)CH₃, —CH(OH)CH₃, and R³ is H, or R¹ is bound to carbon β and is —N(alkyl)₂, R² is —C(O)H, —C(O)OH, —CH₂OH, —C(O)CH₃, —CH(OH)CH₃, and R³ is H,

or R¹ is bound to carbon δ and is —N< which is covalently bound to both carbon α and carbon β and either R² is —H and R³ is —C(O)H, —CH₂OH, -aryl—C(O)H, -aryl—CH₂OH, -aryl-C(O)OH, -alkynyl—C(O)H, -alkynyl—CH₂OH, —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where X is a halide, —C(CHX)(aryl) where X is a halide, —C(═NOH)(aryl), —CH(CH₃)(aryl), —CH₂-(aryl), or —C(CH₂)(aryl); or R³ is —H or X where X is a halide, alkyl, alkenyl, alkoxy, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl; or heteroaryl, R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl,

wherein when R¹ is —N(CH₃)₂ and is bound at carbon δ and R³ is —C(O)CH₃, -alkynyl—C(O)CH₃, -alkynyl—C(O)CH₃, or —CH(OH)(CH₃), or R¹ is —O-alkyl and is bound at carbon δ and R³ is —C(O)H, then R² is OH, a C₂-C₇ alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁴, —CH(OH)R⁴, —R⁵—C(O)R⁴, or —R⁵—CH(OH)R⁴, Y is O, X is O and bond γ is a single bond, and

wherein when R¹ is —N(propyl)₂ wherein one propyl is covalently bound to carbon α and the other propyl is covalently bound to carbon β, and R² is —C(O)CH₃ or —C(O)OH, and R³ is —H, then Y is absent, X is CH and bond γ is a double bond,

wherein when R¹ is —N(propyl)₂ wherein one propyl is covalently bound to carbon α and the other propyl is covalently bound to carbon β, and R³ is -methylcarbonylphenyl, methylhydroxyphenyl, —C≡C—C(O)CH₃, or —C≡C—CH(OH)—CH₃, and R² is —H, then Y is absent, X is CH and bond γ is a double bond,

wherein when R¹ is —OCH₃ and is bound to carbon δ, and R³ is methylcarbonylphenyl, methylhydroxyphenyl, —C≡C—C(O)CH₃, or —C≡C—CH(OH)—CH₃, and R² is —H, then Y is absent, X is CH and bond γ is a double bond,

or a salt or stereoisomer thereof.

This invention provides the instant compound wherein when R¹ is —O—CH₃ and bound at carbon δ and R³ is —C(O)H, —C(O)CH₃ or —CH(OH)(CH₃), Y is absent, X is CH and bond γ is a double bond, then R² is OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R^{4′}, —R⁵—C(O)R⁴, or —R⁵—CH(OH)R⁴, where R⁴′ is ethyl, alkenyl, alkynyl, substituted aryl or unsubstituted aryl.

This invention provides the instant compound wherein when R¹ is bound to carbon α and is —O-alkyl, R² is —CH(OH)CH₃ or —C(O)OH, and R³ is H, then Y is O, X is O, and bond γ is a single bond. This invention provides the instant compound wherein when R¹ is bound to carbon β and is —O-alkyl, R² is —C(O)H, —CH₂OH, —C(O)CH₃ or C(O)OH, and R³ is H, then Y is O, X is O, and bond γ is a single bond. This invention provides the instant compound wherein when R¹ is bound to carbon β and is —N(alkyl)₂, R² is —C(O)H, or —CH₂OH, and R³ is H, then Y is O, X is O, and bond γ is a single bond. This invention provides the instant compound wherein when R¹ is bound to carbon δ and is H, R² is —H or —O—CH₃, and R³ is —C(O)H, then Y is O, X is O, and bond γ is a single bond. This invention provides the instant compound wherein R¹ is bound at carbon δ and is >NH which is covalently bound to carbon α or to carbon β and the nitrogen atom and/or a carbon atom is substituted with one or more of an alkyl, alkylene-X where X is a halide, alkylene-C(O)OH, alkenyl, alkynyl, alkoxy, or alcohol.

This invention provides the instant compound having the structure:

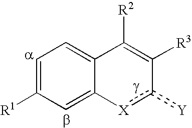

wherein
Y is O, X is O, and bond γ is a single bond, or
Y is absent, X is CH and bond γ is a double bond,

wherein R¹ is —H, —OH, —O-alkyl, —NH-alkyl, —N(alkyl)₂, —NH₂, aryl, heteroaryl, -alkyl—C(O)(OH), -alkyl—OH, or R¹ is >NH which is covalently bound to carbon α or to carbon β; R² is H, OH, a C₂-C₇ alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R6, —CH(OH)R⁴, —R⁵—C(O)R⁴, or —R⁵—CH(OH)R⁴; or R² and R³ together form a ring substituted with ═O,
where R⁴ is methyl, alkenyl, alkynyl, or aryl; R⁵ is alkyl, alkenyl, alkynyl, aryl, or cycloalkyl; and R⁶ is alkenyl, alkynyl, aryl, or cycloalkyl,

or R¹ is —N< which is covalently bound to both carbon α and carbon β and either R² is —H and R³ is —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where X is a halide, —C(CHX)(aryl) where X is a halide, —C(═NOH)(aryl), —CH(CH₃)(aryl), —CH₂-(aryl), or —C(CH₂)(aryl); or R³ is —H and R² is —C(O)R¹¹, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl); or R² and R³ together form a ring substituted with ═O,
where R⁷ is cycloalkyl, C₂-C₇ alkyl, alkenyl, alkynyl, aryl, or heteroaryl; R⁸ is hydroxy, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is hydroxy, alkenyl, alkynyl, aryl, or heteroaryl,

wherein when R¹ is —N(CH₃)₂ and R³ is —C(O)CH₃, alkynyl-C(O)CH₃, or —CH(OH)(CH₃), then R² is OH, a C₂-C₇ alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁴, —CH(OH)R⁴, —R⁵—C(O)R⁴, or —R⁵—CH(OH)R⁴, Y is O, X is O and bond γ is a single bond,

wherein when R¹ is —N(propyl)₂ wherein one propyl is covalently bound to carbon α and the other propyl is covalently bound to carbon β, and R² is —C(O)CH₃ or —CH(OH)CH₃, and R³ is —H, then Y is absent, X is CH and bond γ is a double bond, and

wherein when R¹ is —N(propyl)₂ wherein one propyl is covalently bound to carbon α and the other propyl is covalently bound to carbon β, and R³ is —methylcarbonylphenyl, methylhydroxyphenyl, —C≡C—C(O)CH₃, or —C≡C—CH(OH)—CH₃, and R² is —H, then Y is absent, X is CH and bond γ is a double bond,

wherein when R¹ is —OCH₃, R³ is methylcarbonylphenyl, methylhydroxyphenyl, —C≡C—C(O)CH₃, or —C≡C—CH(OH)—CH₃, and R² is —H, then Y is absent, X is CH and bond γ is a double bond.

This invention provides the instant compound wherein R¹ is —H, —OH, —O-alkyl, —NH-alkyl, —N(alkyl)₂, —NH₂, aryl, heteroaryl, -alkyl-C(O)(OH), -alkyl-OH, or R¹ is >NH which is covalently bound to carbon α or to carbon β, and wherein R² or R³ is —C(O)R⁴, —CH(OH)R⁴, —R⁵—C(O)R⁴, or —R⁵—CH(OH)R⁴; or R² and R³ together form a ring substituted with ═O,
where R⁴ is methyl, alkenyl, alkynyl, or aryl; R⁵ is alkyl, alkenyl, alkynyl, aryl, or cycloalkyl; and R⁶ is alkenyl, alkynyl, aryl, or cycloalkyl,

or R¹ is —N< which is covalently bound to both carbon α and carbon β and either R² is —H and R³ is —C(O)R⁷, —CH(OH)R⁸, —R¹⁰—C(O)R⁹, or —R¹⁰—CH(OH)R⁹; or R³ is —H and R² is —C(O)R¹¹, —CH(OH)R⁷, —R¹⁰—C(O)R⁹, —R¹⁰—CH(OH)R⁹; or R² and R³ together form a ring substituted with ═O,
where R⁷ is cycloalkyl, C₂-C₇ alkyl, alkenyl, alkynyl, aryl, or heteroaryl; R⁸ is hydroxy, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is hydroxy, alkenyl, alkynyl, aryl, or heteroaryl.

This invention provides the instant compound wherein when R¹ is —N(propyl)₂ wherein one propyl is covalently bound to carbon α and the other propyl is covalently bound to carbon β, and R² is —C(O)(phenyl), —C(OH)(phenyl), then R³ is —H, or R² and R³ join together to form a ring substituted with ═O,

wherein when R¹ is —N(CH₃)₂, then R² is —C(O)CH₃, or —CH(OH)CH₃, and R³ is —H, and

wherein when R¹ is —NH which is covalently bound to either carbon α or carbon β, then R² is —C(O)CH₃, or —CH(OH)CH₃, and R³ is —H.

This invention provides the instant compound having the structure:

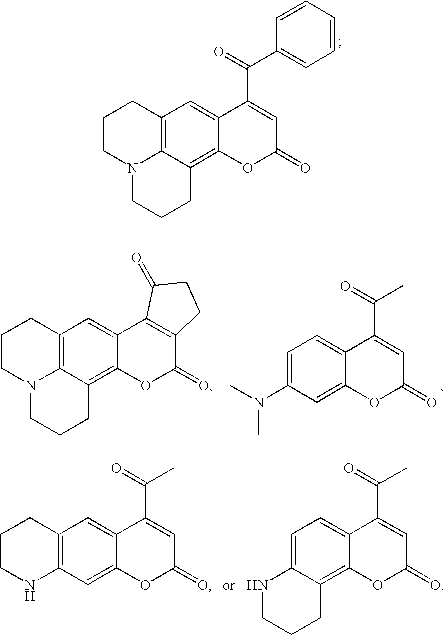

This invention provides the instant compound wherein R¹ is —H, —OH, —O-alkyl, —NH-alkyl, —N(alkyl)₂, —NH₂, aryl, heteroaryl, -alkyl-C(O)(OH), -alkyl-OH, or R¹ is >NH which is covalently bound to carbon α or to carbon β; R² is H, OH, a C₂-C₇ alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, or halide; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide,

or R¹ is —N< which is covalently bound to both carbon α and carbon β and either R² is —H and R³ is —C(CX₂)(aryl) where X is a halide, —C(CX₂)(alkyl) where X is a halide, —C(CHX)(aryl) where X is a halide, —C(═NOH)(aryl), —CH(CH₃)(aryl), —CH₂-(aryl), or —C(CH₂)(aryl); or R³ is —H and R² is —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl).

This invention provides the instant compound having the structure:

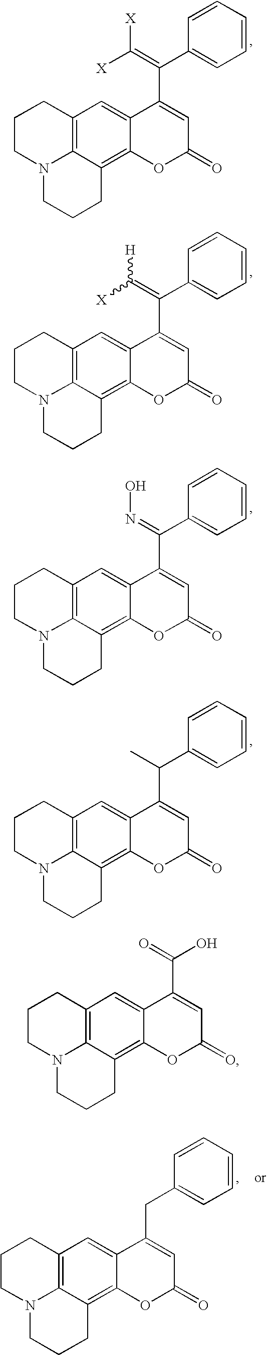

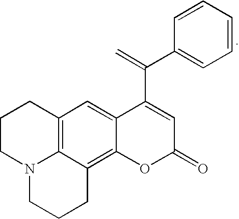

This invention provides the instant compound having the structure:

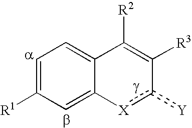

wherein X is CH, Y is CH, and γ is a double bond;

This invention provides the instant compound having the structure:

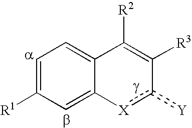

wherein X is O, Y is O, and γ is a single bond, wherein R¹ is —NH-alkyl, —N(alkyl)₂, —NH₂, or R¹ is >NH which is covalently bound to carbon α or to carbon β.

This invention provides the instant compound having the structure:

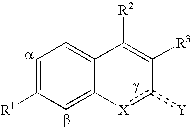

wherein R¹ is —N< which is covalently bound to both carbon α and carbon β, X is O, Y is O, and γ is a single bond.

This invention provides the instant compound having the structure:

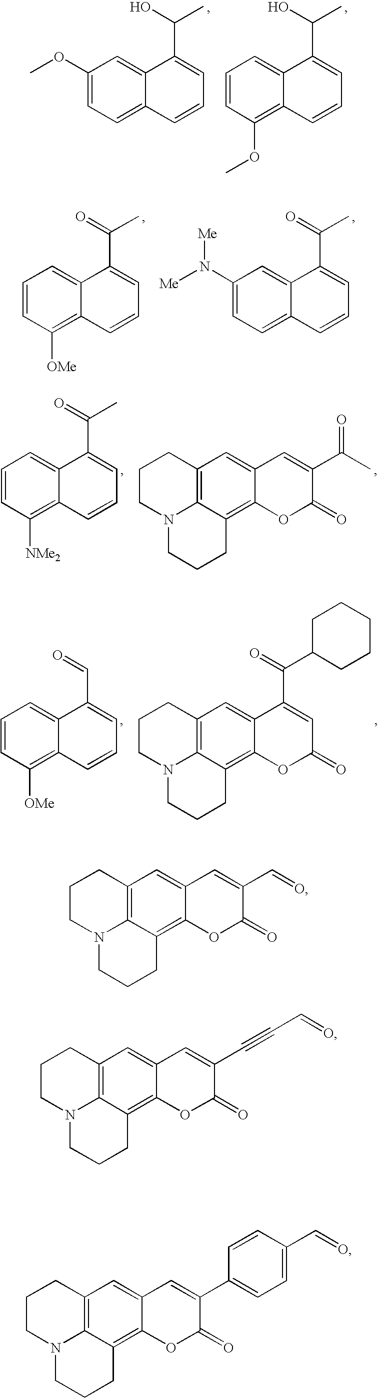

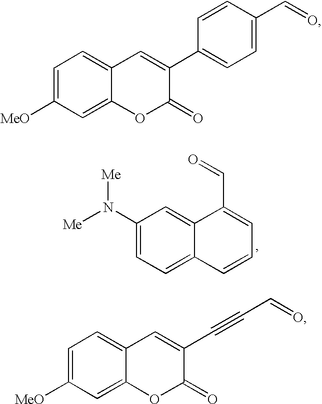

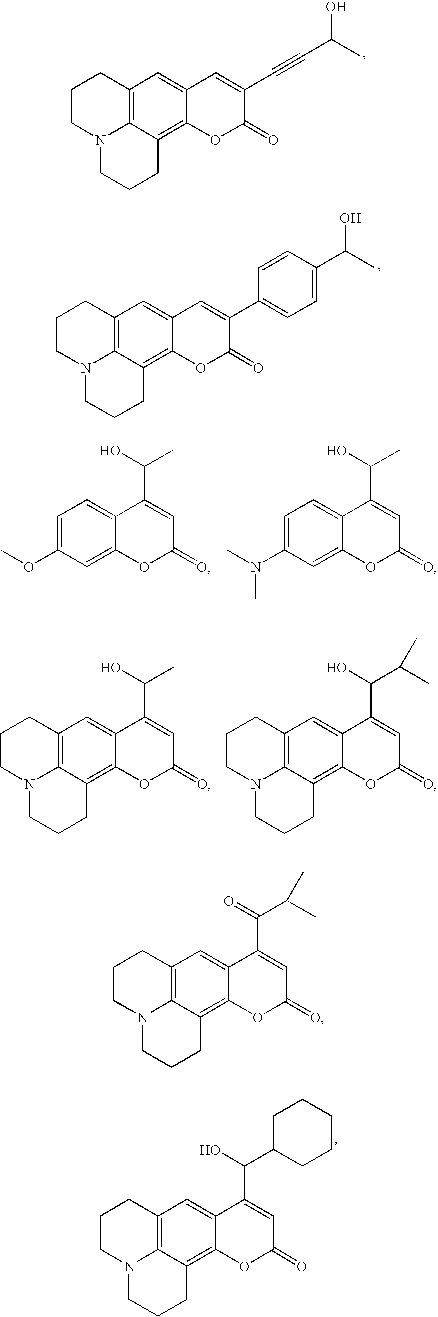

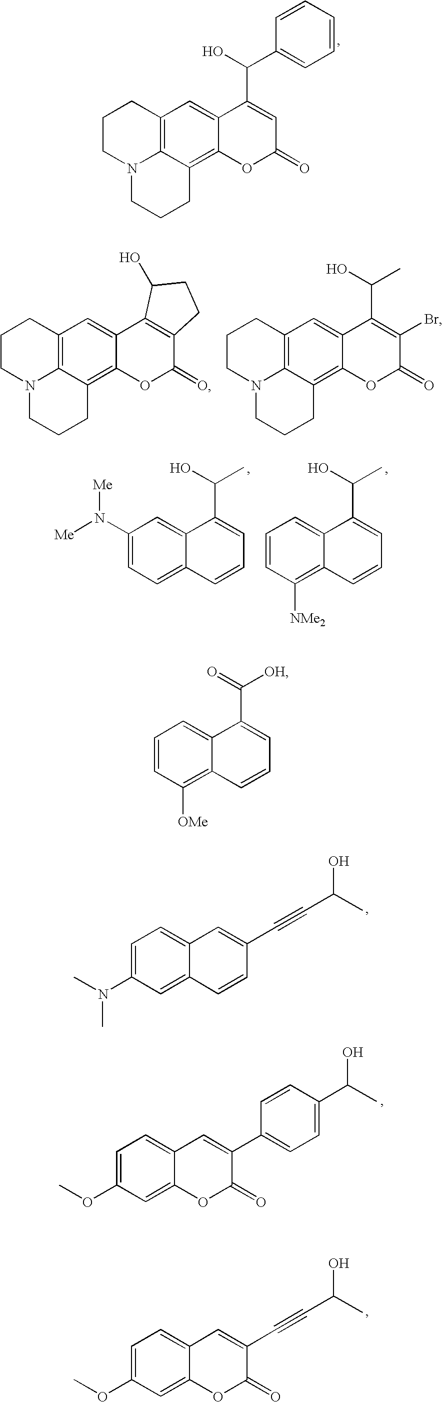

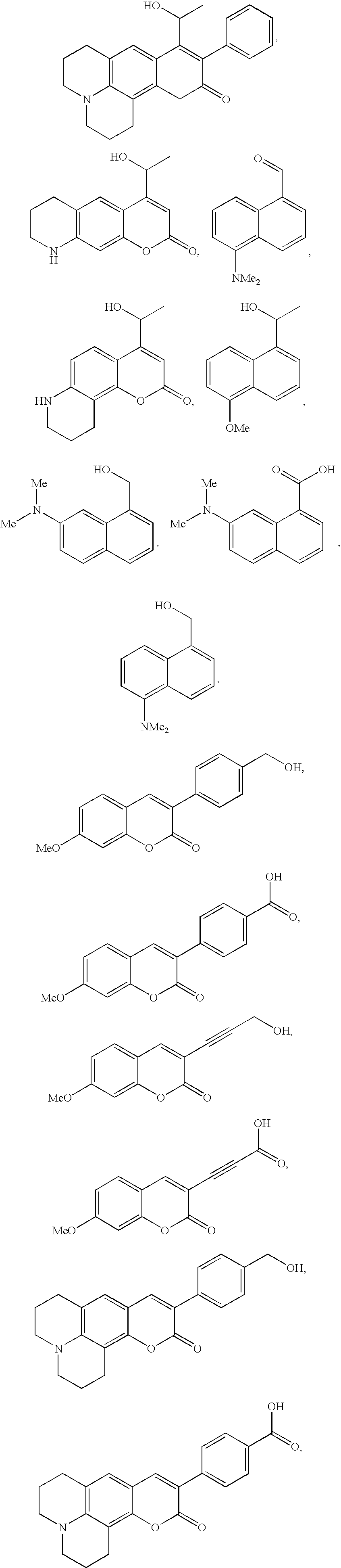

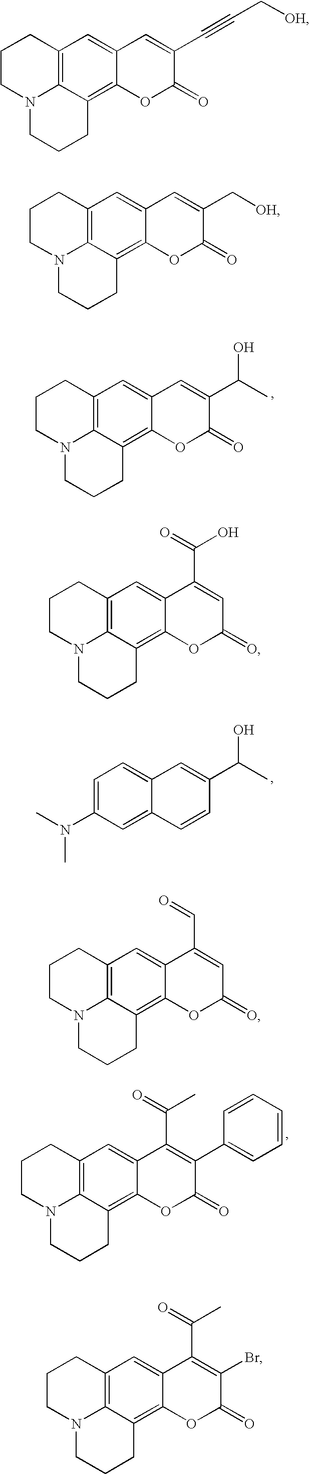

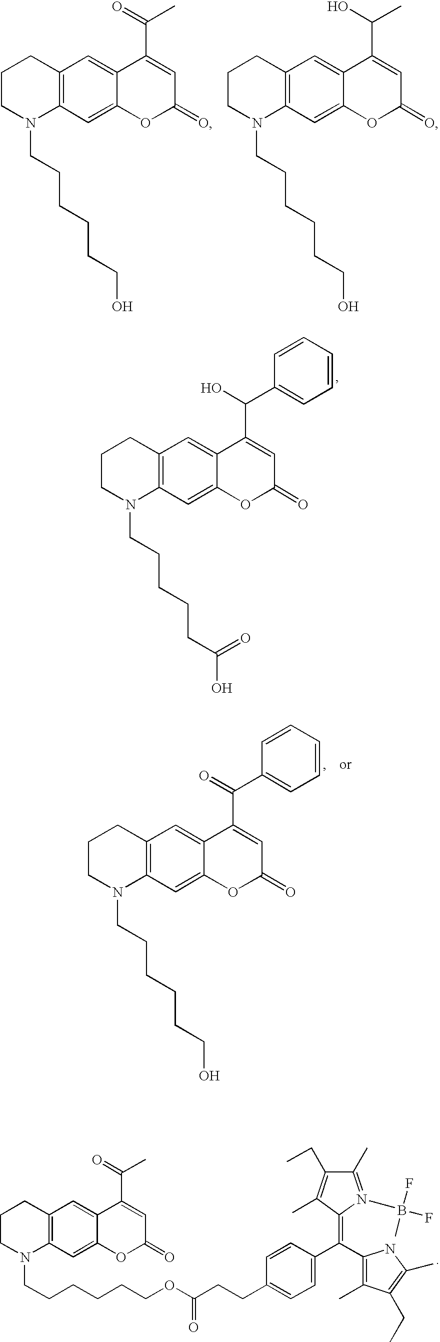

This invention provides a process for preparing the instant compound comprising:
reacting a compound having the structure:

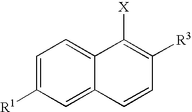

wherein X is —Br, —I, or —OTf
with any one of
   (i) a compound having the structure:

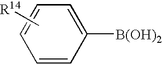

or
(ii) a compound having the structure:

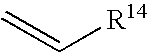

or
(iii) a compound having the structure:

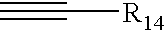

in the presence of palladium of a zero oxidation state to produce a compound having the structure:

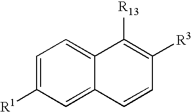

wherein R¹³ is:

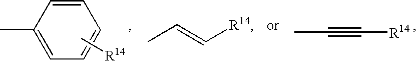

wherein R¹⁴ is any of R² or R³,

wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl—C(O)H, -aryl—CH₂OH, -aryl-C(O)OH, -alkynyl—C(O)H, -alkynyl—CH₂OH, or -alkynyl-C(O)OH; or R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,

or R¹ is bound to carbon α and is —N(alkyl)₂, R² is —C(O)H, —CH₂OH, —C(O)OH, —C(O)CH₃, —CH(OH)CH₃, and R³ is H, or R¹ is bound to carbon α and is —O-alkyl, R² is —CH(OH)CH₃ or —C(O)OH, and R³ is H,

or R¹ is bound to carbon β and is —O-alkyl, R² is —C(O)H, —C(O)OH, —CH₂OH, —C(O)CH₃, —CH(OH)CH₃, and R³ is H,

or R¹ is bound to carbon β and is —N(alkyl)₂, R² is —C(O)H, —C(O)OH, —CH₂OH, —C(O)CH₃, —CH(OH)CH₃, and R³ is H,

or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl—C(O)H, -alkynyl—CH₂OH, —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where —X is a halide, —C(CHX)(aryl) where X is a halide, —C(═NOH)(aryl), —CH(CH₃)(aryl), —CH₂-(aryl), or —C(CH₂)(aryl); or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl.

This invention provides a process for preparing the instant compound comprising:
reacting a compound having the structure:

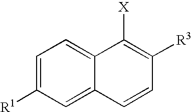

wherein X is —Br, —I, or —OTf
with any one of
   (i) a compound having the structure:

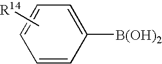

or
(ii) a compound having the structure:

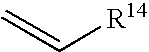

or
(iii) a compound having the structure:

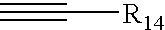

in the presence of palladium of a zero oxidation state to produce a compound having the structure:

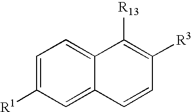

wherein R¹³ is:

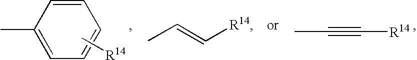

wherein R¹⁴ is any of R² or R³,

wherein R¹ is bound at carbon β and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl—C(O)H, -alkynyl—C(O)OH, or -alkynyl-CH₂OH; or R² and R³ together form a ring substituted with ═O,
where R⁴ is methyl, ethyl, alkenyl, alkynyl, a substituted aryl or an unsubstituted aryl, R⁵ is alkyl, alkenyl, alkynyl, substituted aryl or an unsubstituted aryl, or cycloalkyl, and R⁶ is hydrogen, methyl, a C₃-C₇ alkyl, alkenyl, alkynyl, aryl, or cycloalkyl,

or R¹ is bound to carbon α and is —N(alkyl)₂, R² is —C(O)H, —CH₂OH, —C(O)OH, —C(O)CH₃, —CH(OH)CH₃, and R³ is H,

or R¹ is bound to carbon α and is —O-alkyl, R² is CH(OH)CH₃ or —C(O)OH, and R³ is H,

or R¹ is bound to carbon β and is —O-alkyl, R² is —C(O)H, —C(O)OH, —CH₂OH, —C(O)CH₃, —CH(OH)CH₃, and R³ is H,

or R¹ is bound to carbon β and is —N(alkyl)₂, R² is —C(O)H, —C(O)OH, —CH₂OH, —C(O)CH₃, —CH(OH)CH₃, and R³ is H,

or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl—C(O)H, -alkynyl—CH₂OH, —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where X is a halide, —C(CHX)(aryl) where X is a halide, —C(═NOH)(aryl), —CH(CH₃)(aryl), —CH₂-(aryl), or —C(CH₂)(aryl); or R³ is —H or X where X is a halide, alkyl, alkenyl, alkoxy, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl; or heteroaryl, R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl.

This invention provides a process for preparing the instant compound comprising:
reacting a compound having the structure:

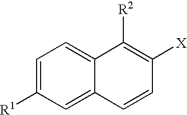

wherein X is —Br, —I, or —OTf
with any one of
   (i) a compound having the structure:

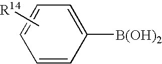

or
(ii) a compound having the structure:

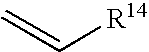

or
(iii) a compound having the structure:

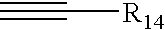

in the presence of palladium of a zero oxidation state to produce a compound having the structure:

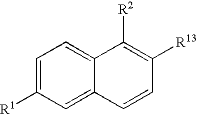

wherein R¹³ is:

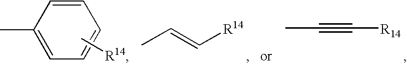

wherein R¹⁴ is any of R² or R³,

wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl—C(O)H, -alkynyl—CH₂OH, or -alkynyl-C(O)OH; or R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,

or R¹ is bound to carbon α and is —N(alkyl)₂, R² is —C(O)H, —CH₂OH, —C(O)OH, —C(O)CH₃, —CH(OH)CH₃, and R³ is H,

or R¹ is bound to carbon α and is —O-alkyl, R² is —CH(OH)CH₃ or —C(O)OH, and R³ is H,

or R¹ is bound to carbon β and is —O-alkyl, R² is —C(O)H, —C(O)OH, —CH₂OH, —C(O)CH₃, —CH(OH)CH₃, and R³ is H,

or R¹ is bound to carbon β and is —N(alkyl)₂, R² is —C(O)H, —C(O)OH, —CH₂OH, —C(O)CH₃, —CH(OH)CH₃, and R³ is H,

or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl—C(O)H, -alkynyl—CH₂OH, —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where —X is a halide, —C(CHX)(aryl) where X is a halide, —C(═NOH)(aryl), —CH(CH₃)(aryl), —CH₂-(aryl), or —C(CH₂)(aryl); or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl.

This invention provides a process for preparing the instant compound comprising:
reacting a compound having the structure:

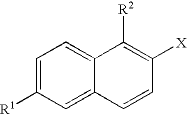

wherein X is —Br, —I, or —OTf
with any one of
   (i) a compound having the structure:

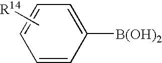

or
(ii) a compound having the structure:

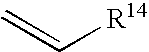

or
(iii) a compound having the structure:

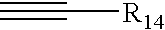

in the presence of palladium of a zero oxidation state to produce a compound having the structure:

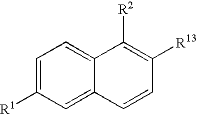

wherein R¹³ is:

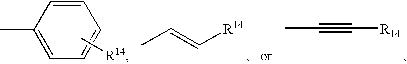

wherein R¹⁴ is any of R² or R³,

wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl—C(O)H, -alkynyl—C(O)OH, or -alkynyl-CH₂OH; or R² and R³ together form a ring substituted with ═O,
where R⁴ is methyl, ethyl, alkenyl, alkynyl, a substituted aryl or an unsubstituted aryl, R⁵ is alkyl, alkenyl, alkynyl, substituted aryl or an unsubstituted aryl, or cycloalkyl, and R⁶ is hydrogen, methyl, a C₃-C₇ alkyl, alkenyl, alkynyl, aryl, or cycloalkyl,

or R¹ is bound to carbon α and is —N(alkyl)₂, R² is —C(O)H, —CH₂OH, —C(O)OH, —C(O)CH₃, —CH(OH)CH₃, and R³ is H,

or R¹ is bound to carbon α and is —O-alkyl, R² is —CH(OH)CH₃ or —C(O)OH, and R³ is H,

or R¹ is bound to carbon β and is —O-alkyl, R² is —C(O)H, —C(O)OH, —CH₂OH, —C(O)CH₃, —CH(OH)CH₃, and R³ is H,

or R¹ is bound to carbon β and is —N(alkyl)₂, R² is —C(O)H, —C(O)OH, —CH₂OH, —C(O)CH₃, —CH(OH)CH₃, and R³ is H,

or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl—C(O)H, -alkynyl—CH₂OH, —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where X is a halide, —C(CHX)(aryl) where X is a halide, —C(═NOH)(aryl), —CH(CH₃)(aryl), —CH₂-(aryl), or —C(CH₂)(aryl); or R³ is —H or X where X is a halide, alkyl, alkenyl, alkoxy, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl; or heteroaryl, R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl.

This invention provides a process for preparing the instant compound comprising:
reacting a compound having the structure:

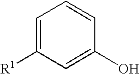

with a compound having the structure:

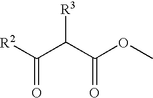

to produce a compound having the structure:

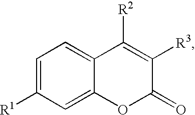

wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl—C(O)H, -alkynyl—CH₂OH, or -alkynyl-C(O)OH; or R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,

or R¹ is bound to carbon α and is —N(alkyl)₂, R² is —C(O)H, —CH₂OH, —C(O)OH, —C(O)CH₃, —CH(OH)CH₃, and R³ is H,

or R¹ is bound to carbon α and is —O-alkyl, R² is —CH(OH)CH₃ or —C(O)OH, and R³ is H,

or R¹ is bound to carbon β and is —O-alkyl, R² is —C(O)H, —C(O)OH, —CH₂OH, —C(O)CH₃, —CH(OH)CH₃, and R³ is H,

or R¹ is bound to carbon β and is —N(alkyl)₂, R² is —C(O)H, —C(O)OH, —CH₂OH, —C(O)CH₃, —CH(OH)CH₃, and R³ is H,

or R¹ is bound to carbon β and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl—C(O)H, -alkynyl—CH₂OH, —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where —X is a halide, —C(CHX)(aryl) where X is a halide, —C(═NOH)(aryl), —CH(CH₃)(aryl), —CH₂-(aryl), or —C(CH₂)(aryl); or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl.

This invention provides a process for preparing the instant compound comprising:
reacting a compound having the structure:

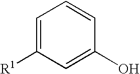

with a compound having the structure:

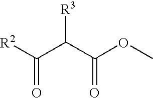

to produce a compound having the structure:

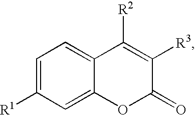

wherein R¹ is bound at carbon β and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl—C(O)H, -alkynyl—C(O)OH, or -alkynyl-CH₂OH; or R² and R³ together form a ring substituted with ═O,
where R⁴ is methyl, ethyl, alkenyl, alkynyl, a substituted aryl or an unsubstituted aryl, R⁵ is alkyl, alkenyl, alkynyl, substituted aryl or an unsubstituted aryl, or cycloalkyl, and R⁶ is hydrogen, methyl, a C₃-C₇ alkyl, alkenyl, alkynyl, aryl, or cycloalkyl,

or R¹ is bound to carbon α and is —N(alkyl)₂, R² is —C(O)H, —CH₂OH, —C(O)OH, —C(O)CH₃, —CH(OH)CH₃, and R³ is H, or R¹ is bound to carbon α and is —O-alkyl, R² is —CH(OH)CH₃ or —C(O)OH, and R³ is H,

or R¹ is bound to carbon β and is —O-alkyl, R² is —C(O)H, —C(O)OH, —CH₂OH, —C(O)CH₃, —CH(OH)CH₃, and R³ is H,

or R¹ is bound to carbon β and is —N(alkyl)₂, R² is —C(O)H, —C(O)OH, —CH₂OH, —C(O)CH₃, —CH(OH)CH₃, and R³ is H,

or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl—C(O)H, -alkynyl—CH₂OH, —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where X is a halide, —C(CHX)(aryl) where X is a halide, —C(═NOH)(aryl), —CH(CH₃)(aryl), —CH₂-(aryl), or —C(CH₂)(aryl); or R³ is —H or X where X is a halide, alkyl, alkenyl, alkoxy, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl; or heteroaryl, R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl.

This invention provides a process for preparing the instant compound comprising:
reacting a compound having the structure:

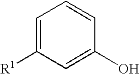

with a compound having the structure:

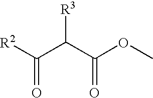

to produce a compound having the structure:

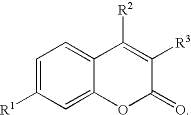

This invention provides a process for preparing the instant compound comprising:
reacting a compound having the structure:

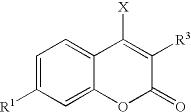

wherein X is —Br, —I, or —OTf
with any one of
   (i) a compound having the structure:

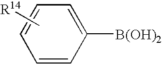

or
(ii) a compound having the structure:

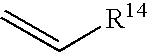

or
(iii) a compound having the structure:

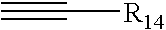

in the presence of palladium of a zero oxidation state to produce a compound having the structure:

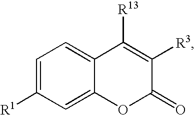

wherein R¹³ is:

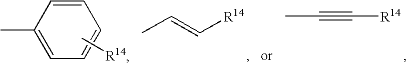

wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl—C(O)H, -alkynyl—CH₂OH, or -alkynyl-C(O)OH; or R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,

or R¹ is bound to carbon α and is —N(alkyl)₂, R² is —C(O)H, —CH₂OH, —C(O)OH, —C(O)CH₃, —CH(OH)CH₃, and R³ is H,

or R¹ is bound to carbon α and is —O-alkyl, R² is —CH(OH)CH₃ or —C(O)OH, and R³ is H,

or R¹ is bound to carbon β and is —O-alkyl, R² is —C(O)H, —C(O)OH, —CH₂OH, —C(O)CH₃, —CH(OH)CH₃, and R³is H,

or R¹ is bound to carbon β and is —N(alkyl)₂, R² is —C(O)H, —C(O)OH, —CH₂OH, —C(O)CH₃, —CH(OH)CH₃, and R³ is H,

or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl—C(O)H, -alkynyl—CH₂OH, —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where —X is a halide, —C(CHX)(aryl) where X is a halide, —C(═NOH)(aryl), —CH(CH₃)(aryl), —CH₂-(aryl), or —C(CH₂)(aryl); or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl.

This invention provides a process for preparing the instant compound comprising:
reacting a compound having the structure:

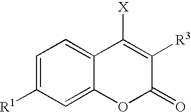

wherein X is —Br, —I, or —OTf
with any one of
   (i) a compound having the structure:

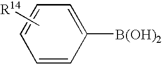

or
(ii) a compound having the structure:

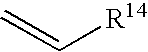

or
(iii) a compound having the structure:

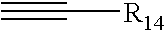

in the presence of palladium of a zero oxidation state to produce a compound having the structure:

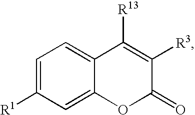

wherein R¹³ is:

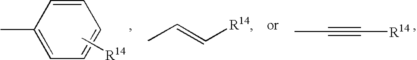

wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl—C(O)H, -alkynyl—C(O)OH, or -alkynyl-CH₂OH; or R² and R³ together form a ring substituted with ═O,
where R⁴ is methyl, ethyl, alkenyl, alkynyl, a substituted aryl or an unsubstituted aryl, R⁵ is alkyl, alkenyl, alkynyl, substituted aryl or an unsubstituted aryl, or cycloalkyl, and R⁶ is hydrogen, methyl, a C₃-C₇ alkyl, alkenyl, alkynyl, aryl, or cycloalkyl,

or R¹ is bound to carbon α and is —N(alkyl)₂, R² is —C(O)H, —CH₂OH, —C(O)OH, —C(O)CH₃, —CH(OH)CH₃, and R³ is H,

or R¹ is bound to carbon α and is —O-alkyl, R² is —CH(OH)CH₃ or —C(O)OH, and R³ is H,

or R¹ is bound to carbon β and is —O-alkyl, R² is —C(O)H, —C(O)OH, —CH₂OH, —C(O)CH₃, —CH(OH)CH₃, and R³ is H,

or R¹ is bound to carbon p and is —N(alkyl)₂, R² is —C(O)H, —C(O)OH, —CH₂OH, —C(O)CH₃, —CH(OH)CH₃, and R³ is H,

or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl—C(O)H, -alkynyl—CH₂OH, —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where X is a halide, —C(CHX)(aryl) where X is a halide, —C(═NOH)(aryl), —CH(CH₃)(aryl), —CH₂-(aryl), or —C(CH₂)(aryl); or R³ is —H or X where X is a halide, alkyl, alkenyl, alkoxy, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl; or heteroaryl, R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl.

This invention provides a process for preparing the instant compound comprising:
reacting a compound having the structure:

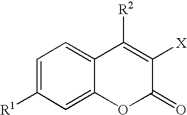

wherein X is —Br, —I, or —OTf
with any one of
   (i) a compound having the structure:

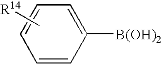

or
(ii) a compound having the structure:

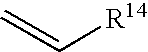

or
(iii) a compound having the structure:

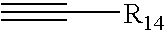

in the presence of palladium of a zero oxidation state to produce a compound having the structure:

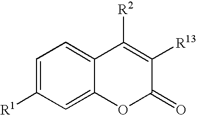

wherein R¹³ is:

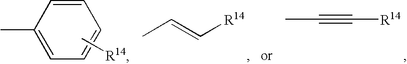

wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH) R⁴, -aryl-C(O)H, -aryl-CH₂OH, -aryl-C(O)OH, -alkynyl—C(O)H, -alkynyl—CH₂OH, or -alkynyl-C(O)OH; or R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,

or R1 is bound to carbon α and is —N(alkyl)₂, R² is —C(O)H, —CH₂OH, —C(O)OH, —C(O)CH₃, —CH(OH)CH₃, and R³ is H,

or R¹ is bound to carbon α and is —O-alkyl, R² is —CH(OH)CH₃ or —C(O)OH, and R³ is H,

or R¹ is bound to carbon β and is —O-alkyl, R² is —C(O)H, —C(O)OH, —CH₂OH, —C(O)CH₃, —CH(OH)CH₃, and R³ is H,

or R¹ is bound to carbon β and is —N(alkyl)₂, R² is —C(O)H, —C(O)OH, —CH₂OH, —C(O)CH₃, —CH(OH)CH₃, and R³ is H,

or R¹ is bound to carbon β and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl—C(O)H, -alkynyl—CH₂OH, —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where —X is a halide, —C(CHX)(aryl) where X is a halide, —C(═NOH)(aryl), —CH(CH₃)(aryl), —CH₂-(aryl), or —C(CH₂)(aryl); or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl.

This invention provides a process for preparing the instant compound comprising:
reacting a compound having the structure:

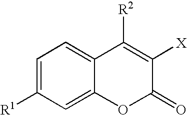

wherein X is —Br, —I, or —OTf
with any one of
   (i) a compound having the structure:

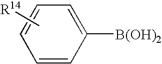

or
(ii) a compound having the structure:

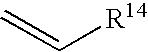

or
(iii) a compound having the structure:

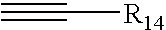

in the presence of palladium of a zero oxidation state to produce a compound having the structure:

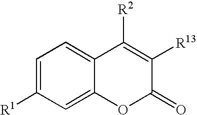

wherein R¹³ is:

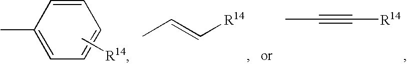

wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl—C(O)H, -alkynyl—C(O)OH, or -alkynyl-CH₂OH; or R² and R³ together form a ring substituted with ═O,
where R⁴ is methyl, ethyl, alkenyl, alkynyl, a substituted aryl or an unsubstituted aryl, R⁵ is alkyl, alkenyl, alkynyl, substituted aryl or an unsubstituted aryl, or cycloalkyl, and R⁶ is hydrogen, methyl, a C₃-C₇ alkyl, alkenyl, alkynyl, aryl, or cycloalkyl,

or R¹ is bound to carbon α and is —N(alkyl)₂, R² is —C(O)H, —CH₂OH, —C(O)OH, —C(O)CH₃, —CH(OH)CH₃, and R³ is H,

or R¹ is bound to carbon α and is —O-alkyl, R² is —CH(OH)CH₃ or —C(O)OH, and R³ is H,

or R¹ is bound to carbon β and is —O-alkyl, R² is —C(O)H, —C(O)OH, —CH₂OH, —C(O)CH₃, —CH(OH)CH₃, and R³ is H,

or R¹ is bound to carbon β and is —N(alkyl)₂, R² is —C(O)H, —C(O)OH, —CH₂OH, —C(O)CH₃, —CH(OH)CH₃, and R³ is H,

or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl—C(O)H, -alkynyl—CH₂OH, —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where X is a halide, —C(CHX)(aryl) where X is a halide, —C(═NOH)(aryl), —CH(CH₃)(aryl), —CH₂-(aryl), or —C(CH₂)(aryl); or R³ is —H or X where X is a halide, alkyl, alkenyl, alkoxy, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl; or heteroaryl, R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl.

This invention provides a process for preparing the instant compound comprising:
reacting a compound having the structure:

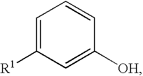

with a compound having the structure:

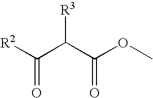

to produce a compound having the structure:

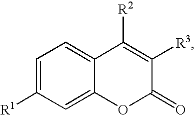

wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl—C(O)H, -alkynyl—CH₂OH, or -alkynyl-C(O)OH; or R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,

or R¹ is bound to carbon α and is —N(alkyl)₂, R² is C(O)H, —CH₂OH, —C(O)OH, —C(O)CH₃, —CH(OH)CH₃, and R³ is H,

or R¹ is bound to carbon α and is —O-alkyl, R² is —CH(OH)CH₃ or —C(O)OH, and R³ is H,

or R¹ is bound to carbon β and is —O-alkyl, R² is —C(O)H, —C(O)OH, —CH₂OH, —C(O)CH₃, —CH(OH)CH₃, and R³ is H,

or R¹ is bound to carbon β and is —N(alkyl)₂, R² is C(O)H, —C(O)OH, —CH₂OH, —C(O)CH₃, —CH(OH)CH₃, and R³ is H,

or R¹ is bound to carbon 5 and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl—C(O)H, -alkynyl—CH₂OH, —C(O)R⁷, CH(OH)R⁸, —R¹⁰—C(O)R⁹, —R¹⁰—CH(OH)R⁹, —C(CX₂)(aryl) where X is a halide, —C(CX₂)(alkyl) where —X is a halide, —C(CHX)(aryl) where X is a halide, —C(═NOH)(aryl), —CH(CH₃)(aryl), —CH₂-(aryl), or —C(CH₂)(aryl); or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, C(═NOH)(aryl), —CH(CH₃)(aryl), —CH₂-(aryl), or C(CH₂)(aryl); or R² and R³ together form a ring substituted with ═O or —OH; or R² is —C(O)CH₃ or CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl.

This invention provides a process for preparing the instant compound comprising:
reacting a compound having the structure:

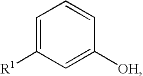

with a compound having the structure:

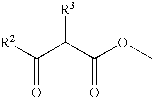

to produce a compound having the structure:

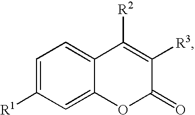

wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl—C(O)H, -alkynyl—C(O)OH, or -alkynyl-CH₂OH; or R² and R³ together form a ring substituted with ═O,
where R⁴ is methyl, ethyl, alkenyl, alkynyl, a substituted aryl or an unsubstituted aryl, R⁵ is alkyl, alkenyl, alkynyl, substituted aryl or an unsubstituted aryl, or cycloalkyl, and R⁶ is hydrogen, methyl, a C₃-C₇ alkyl, alkenyl, alkynyl, aryl, or cycloalkyl,

or R¹ is bound to carbon α and is —N(alkyl)₂, R² is —C(O)H, —CH₂OH, —C(O)OH, —C(O)CH₃, —CH(OH)CH₃, and R³ is H,

or R¹ is bound to carbon α and is —O-alkyl, R² is —CH(OH)CH₃ or —C(O)OH, and R³ is H,

or R¹ is bound to carbon β and is —O-alkyl, R² is —C(O)H, —C(O)OH, —CH₂OH, —C(O)CH₃, —CH(OH)CH₃, and R³ is H,

or R¹ is bound to carbon β and is —N(alkyl)₂, R² is —C(O)H, —C(O)OH, —CH₂OH, —C(O)CH₃, —CH(OH)CH₃, and R³ is H,

or R¹ is bound to carbon δ and is —N< which is covalently bound to both carbon α and carbon β and either R² is —H and R³ is —C(O) H, —CH₂OH, -aryl-C(O)H, -aryl-CH₂OH, -aryl-C(O)OH, -alkynyl—C(O)H, -alkynyl—CH₂OH, —C(O)R⁷, —CH(OH)R⁸, —R¹⁰—C(O)R⁹, —R¹⁰—CH(OH)R⁹, —C(CX₂)(aryl) where X is a halide, —C(CX₂)(alkyl) where X is a halide, —C(CHX)(aryl) where X is a halide, —C(═NOH)(aryl), —CH(CH₃)(aryl), —CH₂-(aryl), or —C(CH₂)(aryl); or R³ is —H or X where X is a halide, alkyl, alkenyl, alkoxy, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂-(aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl; or heteroaryl, R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl.

This invention provides a process for preparing the instant compound comprising:
reacting a compound having the structure:

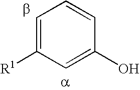

with a compound having the structure:

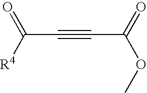

in the presence of triphenylphosphene to produce a compound having the structure:

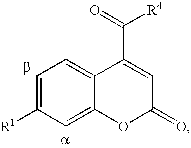

wherein R¹ is —N(CH₃)₂, —N(propyl)₂ wherein one propyl is covalently bound to carbon α and the other propyl is covalently bound to carbon β, or is >NH which is covalently bound to either carbon α or carbon β; and R⁴ is methyl or aryl.

This invention provides a process for preparing the instant compound comprising:
reacting a compound having the structure:

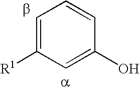

with a compound having the structure:

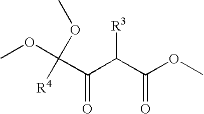

in the presence of Aluminum Chloride (AlCl₃) to produce a compound having the structure:

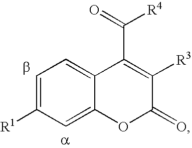

wherein R¹ is —N(CH₃)₂, —N(propyl)₂ wherein one propyl is covalently bound to carbon α and the other propyl is covalently bound to carbon β, R¹ is —N(CH₃)₂, or is >NH which is covalently bound to either carbon α or carbon β, and R⁴ is methyl or aryl.

This invention provides a process for preparing the instant compound comprising:
(a) reacting a compound having the structure:

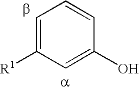

with a compound having the structure:

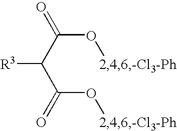

to produce a product;
(b) contacting the product of step (a) with Tf₂O (triflate) and Et₃N (triethylamine) to produce a product;
(c) contacting the product of step (b) with trimethylsilyacetylene, Pd(PPh₃)₂Cl, Copper Iodide and Et₃N to produce a product;
(d) contacting the product of step (c) with K₂CO₃ to produce a product; and
(e) contacting the product of step (d) with H₂O, HgSO₄ and H₂SO₄,
so as to produce a compound having the structure:

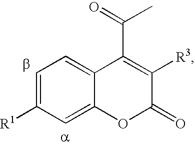

wherein R¹ is —N(propyl)₂ wherein one propyl is covalently bound to carbon α and the other propyl is covalently bound to carbon β, R¹ is —N(CH₃)₂, or is —NH which is covalently bound to either carbon α or carbon β, R³ is H, and R⁴ is methyl or aryl.

This invention provides a composition comprising the instant compound and a pharmaceutically acceptable carrier.

This invention provides a method of identifying a compound not previously known to inhibit human hydroxysteroid dehydrogenase as an inhibitor of human hydroxysteroid dehydrogenase comprising:
a) transfecting a cell which does not express human hydroxysteroid dehydrogenase with a gene encoding for human hydroxysteroid dehydrogenase so that the cell expresses human hydroxysteroid dehydrogenase;
b) providing the cell in a medium;
c) contacting the cell with a reference compound that undergoes a detectable increase in fluorescence when reduced by human hydroxysteroid dehydrogenase under conditions permitting the reference compound to enter the cell;
d) detecting an increase in the fluorescence of the medium;
e) contacting the cell with the compound not previously known to inhibit human hydroxysteroid dehydrogenase under conditions permitting the compound to enter the cell; and
f) detecting a change in the fluorescence of the medium,
wherein a reduced fluorescence of the medium detected in step f) compared to step d) indicates that the compound not previously known to inhibit human hydroxysteroid dehydrogenase is an inhibitor of human hydroxysteroid dehydrogenase, thereby identifying the compound as an inhibitor of human hydroxysteroid dehydrogenase.

This invention provides the instant method wherein the human hydroxysteroid dehydrogenase is aldo-keto reductase 1C1, aldo-keto reductase 1C2, aldo-keto reductase 1C3, or aldo-keto reductase 1C4. This invention provides the instant method wherein the reference compound is one of the instant compounds. This invention provides the instant method wherein the human hydroxysteroid dehydrogenase is aldo-keto reductase 1C3, and the first compound is one of the instant compounds. This invention provides the instant method wherein the human hydroxysteroid dehydrogenase is aldo-keto reductase 1C2, and the first compound is one of the instant compounds.

This invention provides the instant method wherein the cell is a transformed simian cell. This invention provides the instant method wherein the cell is a COS cell.

This invention provides a method of diagnosing a subject as suffering from a cancer of a tissue comprising:
a) obtaining a sample of the tissue which sample comprises a cell of the tissue;
b) providing the sample in a medium;
c) contacting the sample with a compound that undergoes a detectable increase in fluorescence when reduced by human hydroxysteroid dehydrogenase under conditions permitting the compound to enter the cell of the tissue;
d) detecting an increase in the fluorescence of the medium; and
e) comparing the fluorescence detected in step d) with a predetermined fluorescence,
wherein fluorescence of the medium detected in step
d) greater than that of the predetermined fluorescence indicates that the subject is suffering from the cancer of the tissue.

This invention provides the instant method wherein the tissue is prostate tissue or colon tissue and the human hydroxysteroid dehydrogenase is aldo-keto reductase 1C3. This invention provides the instant method wherein the tissue is lung tissue the human hydroxysteroid dehydrogenase is aldo-keto reductase 1C1. This invention provides the instant method wherein the compound is any one of the instant compounds

This invention provides a method of diagnosing a subject as suffering from a cancer of a tissue comprising:
a) obtaining a sample of the tissue which sample comprises a cell of the tissue;
b) obtaining a cellular fraction from the sample;
c) contacting the cellular fraction with a compound that undergoes a detectable increase in fluorescence when reduced by human hydroxysteroid dehydrogenase;
d) detecting an increase in the fluorescence of the cellular fraction; and
e) comparing the fluorescence detected in step d) with a predetermined fluorescence,
wherein fluorescence of the cellular fraction detected in step d) greater than that of the predetermined fluorescence indicates that the subject is suffering from the cancer of the tissue.

This invention provides the instant method wherein the cellular fraction is a whole lysate, a microsomal fraction or a cytosolic fraction. This invention provides the instant method wherein the cellular fraction is a cytosolic fraction. This invention provides the instant method wherein the compound is any one of the instant compounds. This invention provides the instant method wherein the tissue is prostate tissue or colon tissue and the human hydroxysteroid dehydrogenase is aldo-keto reductase 1C3. This invention provides the instant method wherein the human hydroxysteroid dehydrogenase is aldo-keto reductase 1C1, and the tissue is lung tissue.

This invention provides a method of treating a cancer in a subject comprising administering to the cancer in the subject an amount of the compound of any one the instant compounds effective to treat the cancer. This invention provides the instant method wherein the cancer is a prostate cancer, a colon cancer, or a lung cancer.

This invention provides a method of making a composition for use in the treatment of a cancer comprising admixing an effective amount of any one of the instant compounds and a pharmaceutically acceptable carrier.

This invention provides a method of identifying a compound not previously known to inhibit human hydroxysteroid dehydrogenase as an inhibitor of human hydroxysteroid dehydrogenase comprising:
a) providing a human hydroxysteroid dehydrogenase in a medium;
b) contacting the human hydroxysteroid dehydrogenase with a reference compound that undergoes a detectable increase in fluorescence when reduced by human hydroxysteroid dehydrogenase under conditions permitting the reduction of the reference compound by the human hydroxysteroid dehydrogenase;
d) detecting an increase in the fluorescence of the medium;
e) contacting the human hydroxysteroid dehydrogenase with the compound not previously known to inhibit human hydroxysteroid dehydrogenase; and
f) detecting a change in the fluorescence of the medium,
wherein a reduced fluorescence of the medium detected in step f) compared to step d) indicates that the compound not previously known to inhibit human hydroxysteroid dehydrogenase is an inhibitor of human hydroxysteroid dehydrogenase, thereby identifying the compound as an inhibitor of human hydroxysteroid dehydrogenase.

This invention provides the instant method wherein the human hydroxysteroid dehydrogenase is aldo-keto reductase 1C1, aldo-keto reductase 1C2, aldo-keto reductase 1C3, or aldo-keto reductase 1C4. This invention provides the instant method wherein the first compound is any one of the instant compounds. This invention provides the instant method wherein the human hydroxysteroid dehydrogenase is aldo-keto reductase 1C3, and the first compound is of the formula set forth in any one the instant compounds. This invention provides the instant method wherein the human hydroxysteroid dehydrogenase is aldo-keto reductase 1C2, and the first compound is any one of the instant compounds. This invention provides the instant method wherein the human hydroxysteroid dehydrogenase is a component of, or is purified from, a cell lysate. This invention provides the instant method wherein the conditions permitting the reduction of the first compound by the human hydroxysteroid dehydrogenase comprise the presence of NADH or NADPH.

This invention provides a method of identifying a compound not previously known to inhibit human hydroxysteroid dehydrogenase as an inhibitor of human hydroxysteroid dehydrogenase comprising:
a) providing a human hydroxysteroid dehydrogenase in a medium;
b) contacting the human hydroxysteroid dehydrogenase with a reference compound that undergoes a detectable decrease in fluorescence when oxidized by human hydroxysteroid dehydrogenase under conditions permitting the oxidation of the reference compound by the human hydroxysteroid dehydrogenase;
d) detecting an decrease in the fluorescence of the medium;
e) contacting the human hydroxysteroid dehydrogenase with the compound not previously known to inhibit human hydroxysteroid dehydrogenase; and
f) detecting a change in the fluorescence of the medium,
wherein a reduction in the decrease of fluorescence of the medium detected in step f) compared to step
d) indicates that the compound not previously known to inhibit human hydroxysteroid dehydrogenase is an inhibitor of human hydroxysteroid dehydrogenase.

This invention provides a method of identifying a compound not previously known to inhibit human hydroxysteroid dehydrogenase as an inhibitor of human hydroxysteroid dehydrogenase comprising:
a) transfecting a cell which does not express human hydroxysteroid dehydrogenase with a gene encoding for human hydroxysteroid dehydrogenase so that the cell expresses human hydroxysteroid dehydrogenase;
b) providing the cell in a medium;
c) contacting the cell with a reference compound that undergoes a detectable decrease in fluorescence when oxidized by human hydroxysteroid dehydrogenase under conditions permitting the reference compound to enter the cell;
d) detecting a decrease in the fluorescence of the medium;
e) contacting the cell with the compound not previously known to inhibit human hydroxysteroid dehydrogenase under conditions permitting the compound to enter the cell; and
f) detecting a change in the fluorescence of the medium,
wherein a reduction in the decrease of fluorescence of the medium detected in step f) compared to step d) indicates that the compound not previously known to inhibit human hydroxysteroid dehydrogenase is an inhibitor of human hydroxysteroid dehydrogenase.

This invention provides the instant methods wherein the human hydroxysteroid dehydrogenase is a 3α-hydroxysteroid dehydrogenase, a 17β-hydroxysteroid dehydrogenase, or a 20α-hydroxysteroid dehydrogenase.

This invention provides a method of quantitating the amount of a reductase in a sample comprising:
a) providing a sample;
b) contacting the sample with a compound that undergoes a detectable change in fluorescence when reduced by the reductase under conditions permitting reduction;
c) detecting a change in the fluorescence of the sample; and
d) quantifying the amount of reductase in the sample by comparing the fluorescence detected in step c) against a predetermined relationship between fluorescence and reductase amount.

This invention provides a method of quantitating the amount of an oxidase in a sample comprising:
a) providing a sample;
b) contacting the sample with a compound that undergoes a detectable change in fluorescence when oxidized by an oxidase under conditions permitting oxidation;
c) detecting a change in the fluorescence of the sample; and
d) quantifying the amount of oxidase in the sample by comparing the fluorescence detected in step c) against a predetermined relationship between fluorescence and oxidase amount.

This invention provides the instant methods wherein the compound is any one of the instant compounds. This invention provides the instant methods wherein predetermined relationship is a calibration curve determined by plotting fluorescence versus a plurality of product concentrations. This invention provides the instant method wherein the product is an alcohol or a carboxylic acid. This invention provides the instant method wherein the predetermined relationship is a calibration curve determined by plotting fluorescence versus a plurality of starting compound concentrations. This invention provides the instant method wherein the starting compound is a ketone or an aldehyde. This invention provides the instant method wherein the oxidase or reductase is a hydroxysteroid dehydrogenase. This invention provides the instant method wherein the alcohol dehydrogenase is a human hydroxysteroid dehydrogenase. This invention provides the instant method wherein the human hydroxysteroid dehydrogenase is aldo-keto reductase 1C1, aldo-keto reductase 1C2, aldo-keto reductase 1C3, or aldo-keto reductase 1C4. This invention provides the instant method wherein the conditions permitting reduction comprise presence of NADH or NADPH. This invention provides the instant method wherein the sample is an in vitro solution, a cell, a cell lysate, a tissue, or a tissue homogenate. This invention provides the instant methods wherein the compound is any one of the instant compounds.

This invention also provides a composition comprising any one or more of the competitive inhibitor compounds and a pharmaceutically acceptable carrier.

This invention further provides the instant methods wherein the human hydroxysteroid dehydrogenase is aldo-keto reductase 1C3, and the first compound is of the formula set forth in 5c, 5g, or 5h of table 5. This invention also provides the instant method wherein the human hydroxysteroid dehydrogenase is aldo-keto reductase 1C3, and the first compound is of the formula set forth in 5c of table 5. This invention also provides the instant method wherein the human hydroxysteroid dehydrogenase is aldo-keto reductase 1C2, and the first compound is of the formula set forth in 5i of table 5. This invention also provides the instant method wherein the human hydroxysteroid dehydrogenase is a component of, or is purified from, a cell lysate.

Fluorescence measured from tested samples can be compared to predetermined fluorescence as measured from one or more standard samples (i.e. non-cancerous). The predetermined fluorescence is determined under the same conditions as the test sample fluorescence is determined, and for the same tissue type as the tested sample tissue. In addition, the predetermined fluorescence can be a normalized fluorescence of multiple measurements in samples from one or more subjects. In the case of one subject, the non-cancerous standard sample may be from a non-cancerous section of tissue of the same subject as the suspected cancerous sample. In one embodiment the predetermined fluorescence is a normalized fluorescence of multiple non-cancerous tissue samples obtained by averaging the fluorescence values of the samples as quantified under the same conditions that the test sample fluorescence is quantified. In differing embodiments the presence of a cancerous sample is indicated by the test fluorescence being 1%, 2% or n % greater than the predetermined fluorescence, wherein n is any integer between 2 and 1000, or n is an integer greater than 999.

This invention further provides the instant methods, wherein the cancer is a prostate cancer, a myeloid cell cancer, a colon cancer, or a lung cancer. In one embodiment the cancer is a myeloid cell cancer and the compound is a competitive inhibitor of human AKR 1C3. In a further embodiment the myeloid cell cancer is acute myeloid leukemia.

This invention provides the instant methods, wherein the compound is one of the instant compounds. In one embodiment, the predetermined relationship is a calibration curve determined by plotting fluorescence versus a plurality of product concentrations. In an embodiment, the product is an alcohol or a carboxylic acid. In another embodiment, the predetermined relationship is a calibration curve determined by plotting fluorescence versus a plurality of starting compound concentrations. In an embodiment, the starting compound is a ketone or an aldehyde.

As used herein, “AKR” means aldoketoreductase. The terms “aldo-keto reductase” and “aldoketo reductase” are synonymous with aldoketoreductase.

As used herein, “hydroxysteroid dehydrogenase” includes, without limitation, short chain dehydrogenase reductases, 3α-hydroxysteroid dehydrogenases, 20α-hydroxysteroid dehydrogenases, and 17β-hydroxysteroid dehydrogenases.

As used herein, “reference standard” means a normalized value obtained from a normal sample, and in the case of fluorescence means the normalized fluorescence measured form a non-cancerous or other standardized sample as measured by a parallel assay with the same steps and conditions to which the tested or cancerous sample is being subjected.

As used herein, a “competitive inhibitor” in relation to an enzyme is a substance capable of binding to the enzyme's active site in place of the physiological substrate.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. For example, an amount effective to delay the growth of or to cause a cancer to shrink or not metastasize. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

As used herein, the “cancer” of a tissue refers to cancers where human aldo-keto reductase 1Cs activities are enhanced beyond the activity of that enzyme in a non-pathological cell of that tissue. Non-limiting examples of the cancers are prostate, lung, and colon cancer.

As used herein, “diagnosing” a cancer means identifying a cell or a tissue as cancerous, in any cancerous stage, or as predisposed to cancer, based on detecting over-expression of aldo-keto reductase 1Cs, including specific isoforms, or detection of an aldo-keto reductase 1C isoform enzyme activity level enhanced beyond the level of activity of that enzyme in a non-pathological or non-cancerous cell of that tissue.

As used herein, “treatment” of a cancer encompasses inducing inhibition, regression, or stasis/prevention of metastasis of a cancer. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “salt” is salt of the instant compounds which has been modified by making acid or base salts of the compounds. In the case of compounds used for treatment of cancer, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutical carrier.

As used herein “medium” shall include any physiological medium or artificial medium of that supports hydroxysteroid dehydrogenase activity, whether the hydroxysteroid dehydrogenase is cellular or is contained within a lysate or in a purified form. Preferably, the fluorescence of the medium should be negligible or constant.

As used herein, a “reduction” when pertaining to fluorescence can mean either a reduction in the absolute amount of fluorescence, or a reduction in the rate of change of fluorescence, whether the rate of change be positive or negative.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds may comprise a single compound or mixtures thereof with other anti-cancer compounds, other cancer or tumor growth inhibiting compounds. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection or other methods, into the cancer, all using dosage forms well known to those of ordinary skill in the pharmaceutical arts.

The compounds can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone but are generally mixed with a pharmaceutically acceptable carrier. This carrier can be a solid or liquid, and the type of carrier is generally chosen based on the type of administration being used. The carrier can be a monoclonal antibody. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Specific examples of pharmaceutical acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modem Pharmaceutics Drugs and the Pharmaceutical Sciences, Vol 40 (Gilbert S. Banker, Christopher T. Rhodes, Eds.).

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds can also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The active ingredient can be administered orally in solid dosage forms, such as capsules, tablets, and powders, or in liquid dosage forms, such as elixirs, syrups, and suspensions. It can also be administered parentally, in sterile liquid dosage forms.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The instant compounds may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

The present invention also includes pharmaceutical kits useful, for example, for the treatment of cancer, which comprise one or more containers containing a pharmaceutical composition comprising an effective amount of one or more of the compounds. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 . . . , n-1 or n carbons in a linear or branched arrangement. For example, C₁-C₆, as in “C₁-C₆ alkyl” is defined to include groups having 1, 2, 3, 4, 5, or 6 carbons in a linear or branched arrangement, and specifically includes methyl, ethyl, propyl, butyl, pentyl, hexyl, and so on. “Alkoxy” represents an alkyl group of indicated number of carbon atoms attached through an oxygen bridge.

The term “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

If no number of carbon atoms is specified, the term “alkenyl” refers to a non-aromatic hydrocarbon radical, straight or branched, containing at least I carbon to carbon double bond, and up to the maximum possible number of non-aromatic carbon-carbon double bonds may be present. For example, “C₂-C₆ alkenyl” means an alkenyl radical having 2, 3, 4, 5, or 6 carbon atoms, and 1, 2, 3, 4, or 5 carbon-carbon double bonds respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated.

The term “cycloalkenyl” shall mean cyclic rings of 3 to 10 carbon atoms and at least 1 carbon to carbon double bond (i.e., cycloprenpyl, cyclobutenyl, cyclopenentyl, cyclohexenyl, cycloheptenyl or cycloocentyl).

The term “alkynyl” refers to a hydrocarbon radical straight or branched, containing at least 1 carbon to carbon triple bond, and up to the maximum possible number of non-aromatic carbon-carbon triple bonds may be present. Thus, “C₂-C₆ alkynyl” means an alkynyl radical radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated.

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic or bicyclic ring of up to 10 atoms in each ring, wherein at least one ring is aromatic and contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

As appreciated by those of skill in the art, “halo” or “halogen” as used herein is intended to include chloro, fluoro, bromo and iodo.

The term “heterocycle” or “heterocyclyll” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

The alkyl, alkenyl, alkynyl, cycloalkyl, aryl, heteroaryl and heterocyclyl substituents may be unsubstituted or unsubstituted, unless specifically defined otherwise. For example, a (C1-C6)alkyl may be substituted with one or more substituents selected from OH, oxo, halogen, alkoxy, dialkylamino, or heterocyclyl, such as morpholinyl, piperidinyl, and so on.

In the compounds of the present invention, alkyl, alkenyl, alkynyl, cycloalkyl, cycloalkenyl, heterocyclyl and heteroaryl groups can be further substituted by replacing one or more hydrogen atoms be alternative non-hydrogen groups. These include, but are not limited to, halo, hydroxy, mercapto, amino, carboxy, cyano and carbamoyl.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing compounds of the present invention, one of ordinary skill in the art will recognize that the various substituents, i.e. R1, R2, R′, R″, and R are to be chosen in conformity with well-known principles of chemical structure connectivity.

The compounds of the present invention are available in racemic form or as individual enantiomers. For convenience, some structures are graphically represented as a single enantiomer but, unless otherwise indicated, is meant to include both racemic and enantiomerically pure forms. Where cis and trans sterochemistry is indicated for a compound of the present invention, it should be noted that the stereochemistry should be construed as relative, unless indicated otherwise. For example, a (+) or (−) designation should be construed to represent the indicated compound with the absolute stereochemistry as shown.

Racemic mixtures can be separated into their individual enantiomers by any of a number of conventional methods. These include, but are not limited to, chiral chromatography, derivatization with a chiral auxiliary followed by separation by chromatography or crystallization, and fractional crystallization of diastereomeric salts. Deracemization procedures may also be employed, such as enantiomeric protonation of a pro-chiral intermediate anion, and the like.

The methods of the present invention when pertaining to cells, and samples derived or purified therefrom, including enzyme containing fractions, may be performed in vitro. The methods of treatment may, in different embodiments, be performed in vivo, in situ, or in vitro. The methods of diagnosis may, in different embodiments, be performed in vivo, in situ, or in vitro.

The compounds disclosed herein that change their fluorescence characteristics after being reduced or oxidized are useful as competitive substrates for, inter alia, determining the expression level of enzymes in vitro, in situ in cells, in homogenates and cell lysates, and in tissue samples. For example, compounds disclosed here that are reduced by alcohol dehydrogenase to a corresponding fluorescent alcohol are useful for determining the level of alcohol dehydrogenase expression in a sample. A “competitive substrate” in relation to an enzyme is a substance capable of binding to the enzyme's active site in place of the physiological substrate and being converted to product.

The compounds disclosed here that can compete with the physiological substrate for the enzyme's active site are useful as inhibitors of the enzyme's activity on the physiological substrate.

This invention will be better understood by reference to the Experimental Details which follow, but those skilled in the art will readily appreciate that the specific experiments detailed are only illustrative of the invention as described more fully in the claims which follow thereafter.

Experimental Details

Preparation of Suitable Probes:

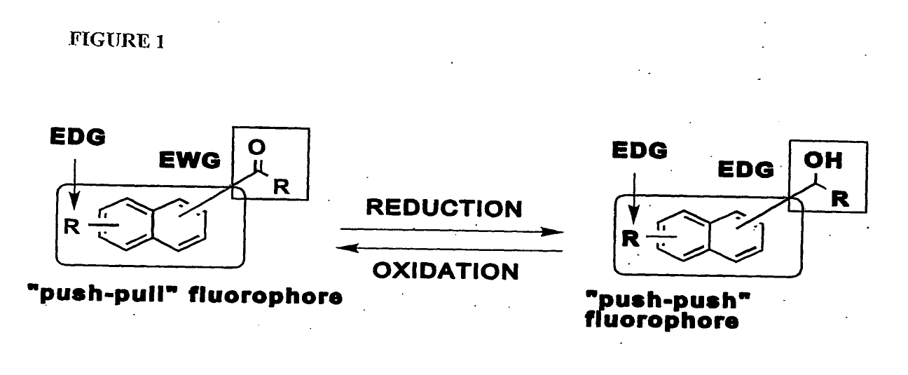

Many organic fluorophores are based on the “push-pull” structural feature wherein an electron-donating and electron-withdrawing groups are electronically connected via an extended π-conjugated system (“push-pull” system) (Rettig, W. Angew. Chem. Xnt. Ed. 1986, 25, 971-988) This class of fluorophores seemed particularly suitable for design of redox probes wherein the ketone carbonyl would be a part of the “push-pull” system. Reduction of the carbonyl group to an alcohol converts an electron-withdrawing group (and often a quenching group) to an electron-donating group, resulting in a profound electronic change of the system, which in turn may lead to a change in the emission profile (FIG. 1) (Previous examples of carbonyl-alcohol fluorogenic probes suffered from short excitation/emission wavelengths in the near UV region. See (a) Wierzchowski, J.; Dafeldecker, W. P.; Holmquist, B.; Vallee, B. L. Anal. Biochem. 1989, 178, 57-62. (b) List, B.; Barbas III, C. F.; Lerner, R. A. Proc. Natl. Acad. Sci. USA 1998, 95, 15351-15355).

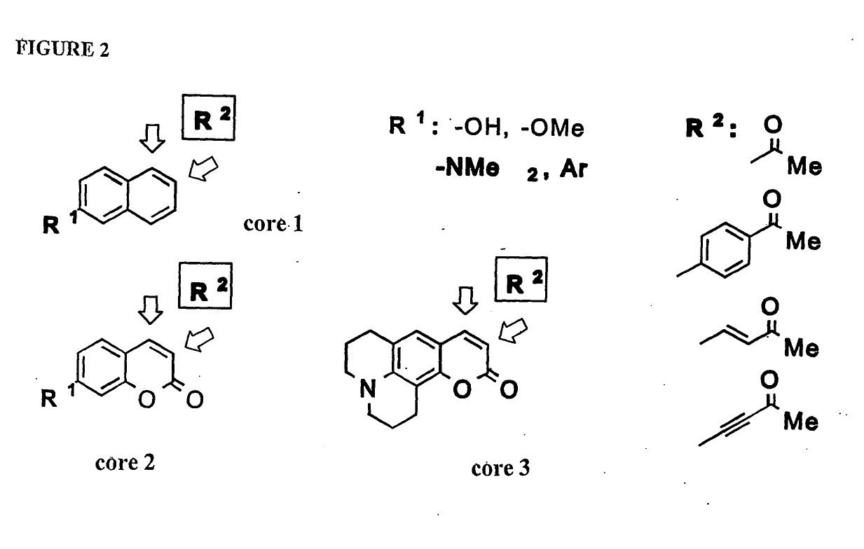

An array of compounds was synthesized according to the design shown in FIG. 1, founded on three aromatic cores (FIG. 2). The ketone group was attached to the core at two positions either directly or via a linker. The linker (benzene, alkene, and alkyne) was introduced to explore the consequences of spatial separation of the ketone and the fluorophore while maintaining the conjugation between these two components. Specifically, the effect of length and nature of the π-conjugation system on emission properties and on the enzyme activity and selectivity (accessibility of the carbonyl group to the enzyme active site) were investigated. A general synthesis schemes is shown below:

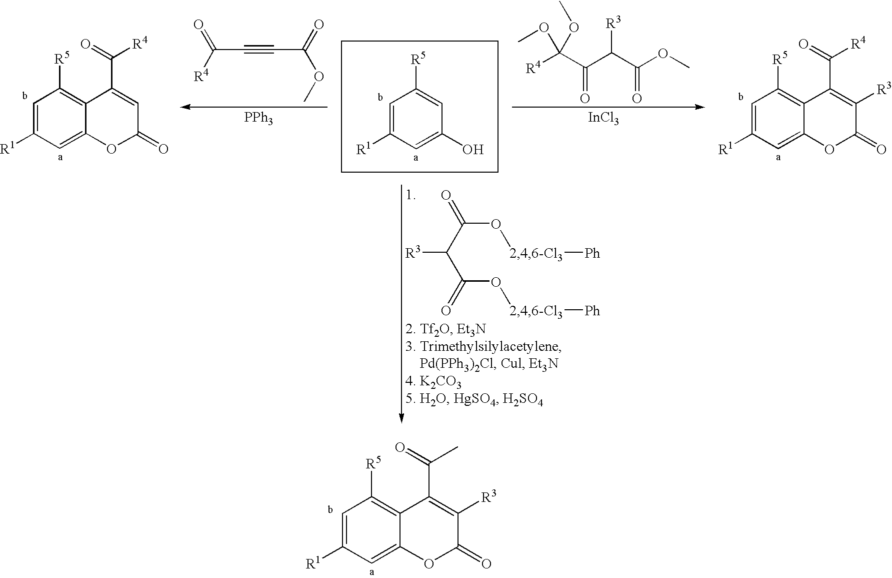

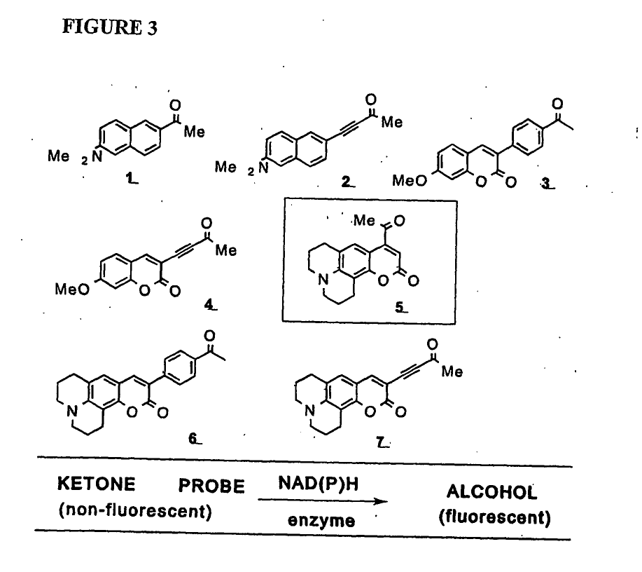

Approximately fifty compounds were synthesized and evaluated in terms of the following physical and chemical properties in an aqueous solution: (1) emission switching between the oxidized (ketone) and reduced form (alcohol); (2) emission wavelength (λₑₘ>430 nm) and quantum yield (Φ>0.1); (3) photochemical stability and chemical stability (including stability to intracellular reductants). Following these strict criteria, seven fluorogenic probes (FIG. 3) were identified with suitable properties; in all cases (except for probe 1) the alcohols were highly fluorescent while the corresponding ketones showed only background level of emission, thus constituting an optical redox switch (FIG. 3). The selected candidates contained three different cores and a variety of linkers, increasing the structural diversity of the set. (See Table 1, and Materials and Methods regarding excitation properties).

TABLE 1
Photochemical Characterization of Compounds

| | Abs. | Fluor. | | Abs. | Fluor. |
|---|---|---|---|---|---|
| | Max | Max | | Max | Max |
| Ketone | (nm) | (nm) | Alcohol | (nm) | (nm) |
| 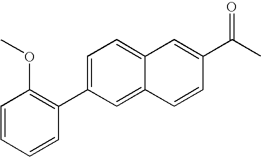 | 312 | 349 | 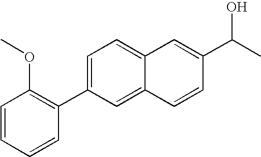 | 274 | 366 |
| 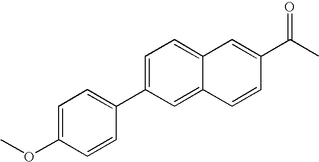 | 316 | 354 | 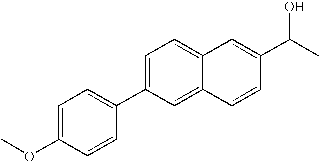 | 279 | 380 |
| 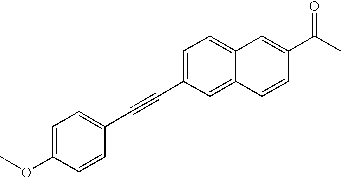 | 334 | 454 | 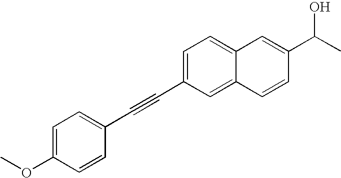 | 346 | 411# |
| 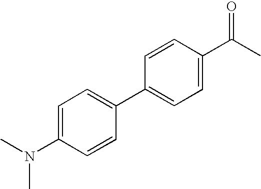 | 360 | 410 | 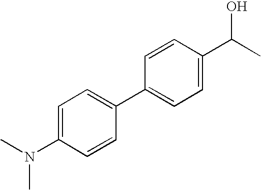 | 295 | 404 |
| 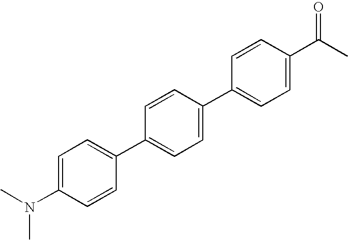 | 346 | 392 | 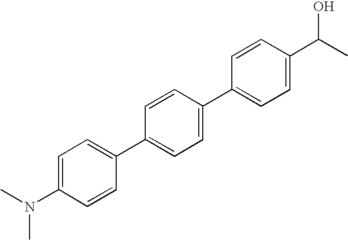 | 318 | 474 |
| 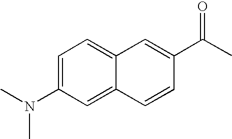 | 368 | 521 | 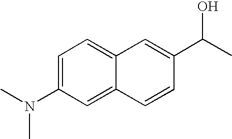 | 348 | 429 |
| 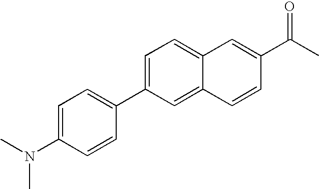 | 361 | — | 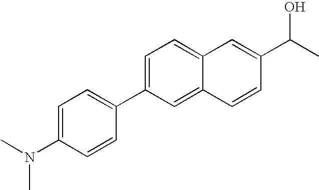 | 316 | 498 |
| 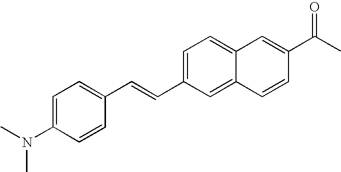 | 396 | 416 | 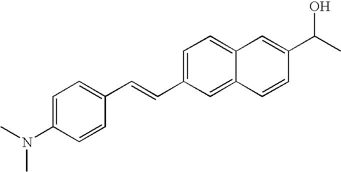 | 347 | 512# |
| 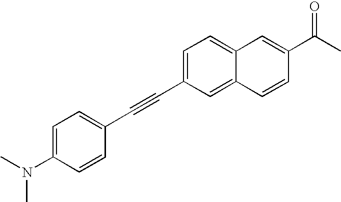 | 377 | 516 | 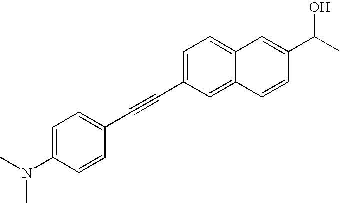 | 335 | 461 |
| 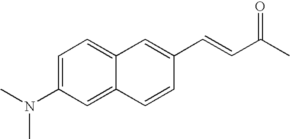 | 395 | — | 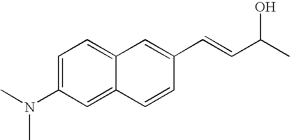 | 318 | 453 |
| 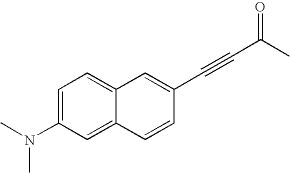 | 389 | 448 | 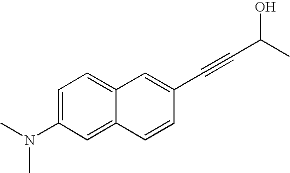 | 361 | 440 |
| 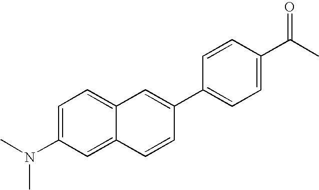 | 364 | — | 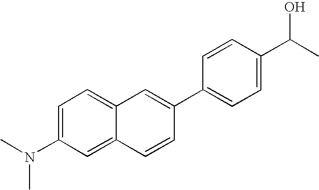 | 315 | 447 |
| 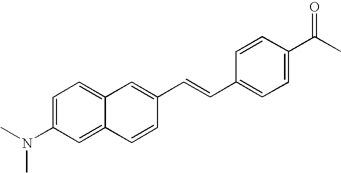 | 392 | 452 | 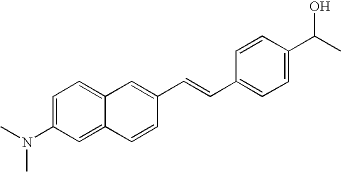 | 348 | 510# |
| 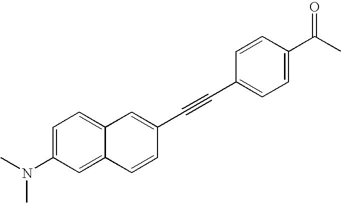 | 378 | — | 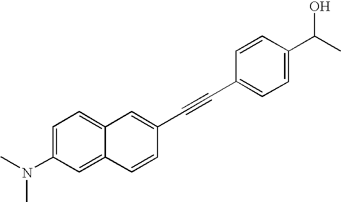 | 331 | — |
| 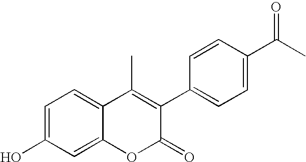 | 333 | 668 | 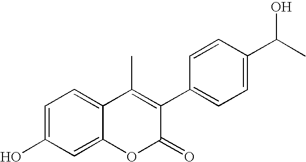 | 329 | 461 |
| 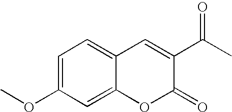 | 359 | 429 | 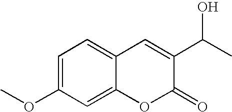 | 320 | 400 |
| 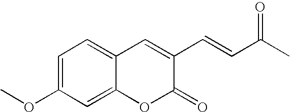 | 322 | 443 | 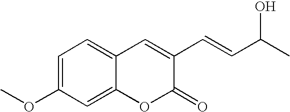 | 278 | 455 |
| 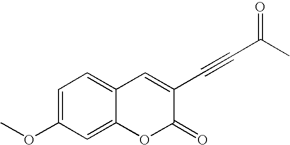 | 368 | 416 | 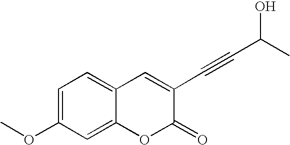 | 346 | 420 |
| 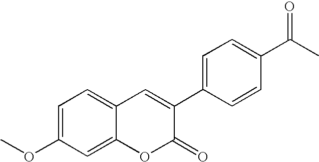 | 348 | 462 | 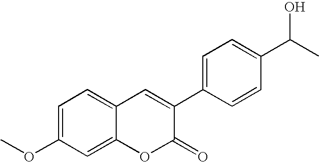 | 342 | 429 |
| 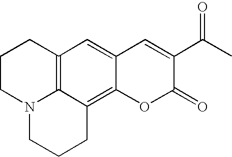 | 449 | 504 | 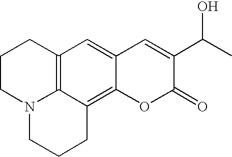 | 378 | 501* |
| 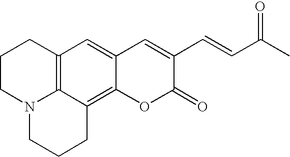 | 465 | 587 | 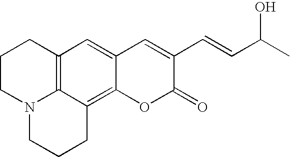 | 416 | 519\hat{ } |
| 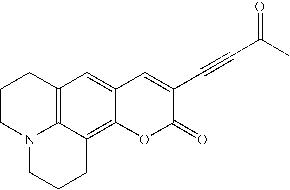 | 464 | 512 | 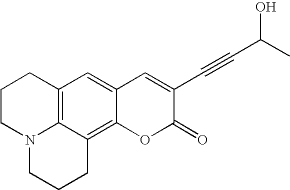 | 429 | 508 |
| 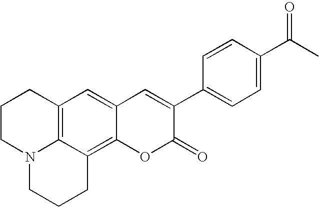 | 435 | 511 | 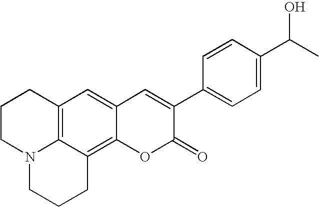 | 422 | 509 |
| 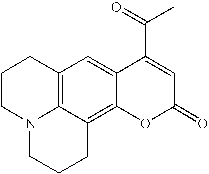 | 418 | 520 | 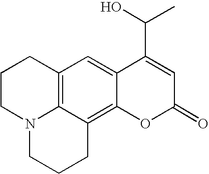 | 398 | 509 |
| 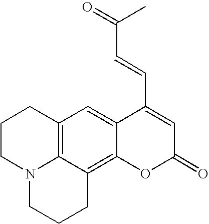 | — | — | 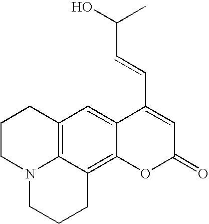 | 402 | 502# |
| 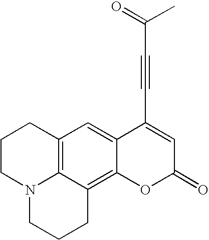 | 458 | 539 | 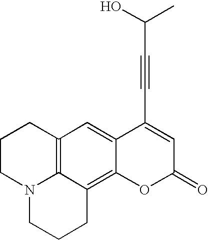 | 433 | 450\hat{ } |
| 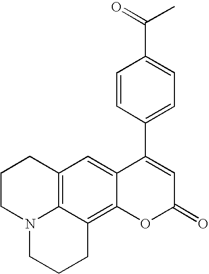 | 410 | 474 | 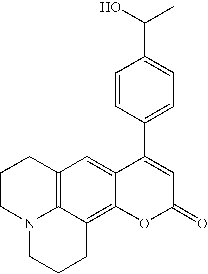 | 405 | 550# |

#low quantum yield,
\hat{ }reactivity with cellular reductants,
*no change in wavelength of emission

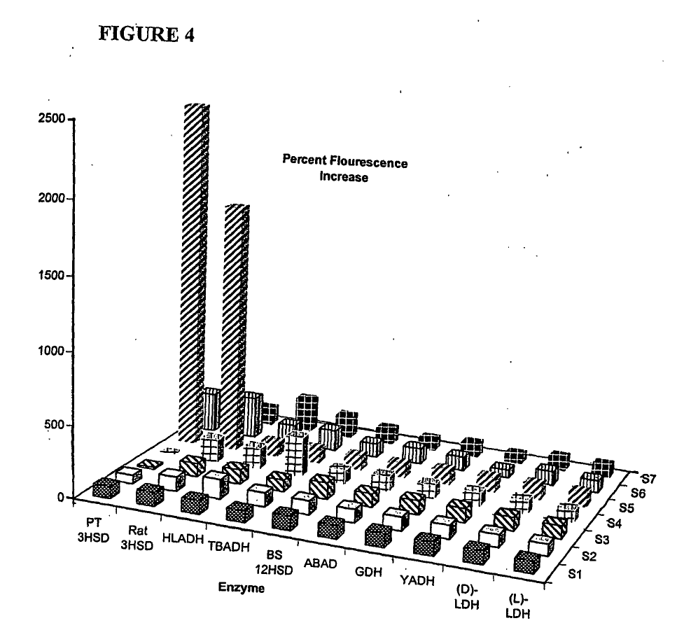

Probes 1-7 (see FIG. 3) were subsequently tested against a collection of dehydrogenases in the presence of NAD(P)H; the extent of reduction was assessed by the measurement of fluorescence intensity at the emission maximum of each probe (FIG. 4). This assay included enzymes from two major oxidoreductase superfamilies, the short chain alcohol dehydrogenases (SDR) and the aldo-keto reductases (AKR), ranging from bacterial to mammalian and human enzymes.

Probe 5 (λₑₘ=510 nm for the corresponding alcohol) was converted rapidly and selectively by 3α-hydroxysteroid dehydrogenases (3α-HSD), namely the bacterial (Pseudomonas) and the rat liver enzymes (FIG. 4).

No other enzymes examined in this assay catalyzed the reduction of probe 5. Similarly, both 3α-HSD enzymes demonstrated high selectivity for 5 among the tested probes. Probe 6 showed good conversion, however at significantly slower rate in comparison to probe 5 (FIG. 4). Surprisingly, horse liver alcohol dehydrogenase (HLAD) and Thermoanaerobium brockii alcohol dehydrogenase (TBAD), both well known for their substrate promiscuity, were not acceptant of probe 5. In contrast, these two latter enzymes catalyzed reduction of alkynyl-ketone probes 4 and 7.

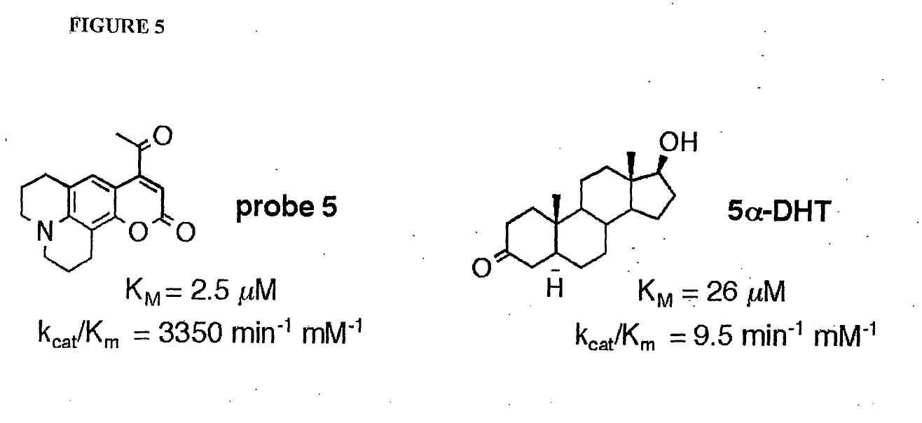

Whether the activity of human enzymes may be imaged by probe 5 was investigated. Type 2 isozyme of 3α-HSD (AKR 1C3) was selected for this study owing to its important physiological role. Probe 5 was rapidly converted by this enzyme and the subsequent quantitative measurements afforded the kinetic parameters (Km=2.5 μM, kcat=8.2 min-1). Remarkably, comparison to 5α-dihydrotestosterone (5α-DHT, Kₘ=26 μM, k_{cat}=0.25 min-1, FIG. 5), a likely physiological substrate in prostate, revealed that synthetic probe 5 is in fact a far better substrate for this enzyme.

Materials and Methods

Spectra

¹H and ¹³C NMR spectra were recorded on Bruker 300 or 400 Fourier transform NMR spectrometers. Spectra were recorded in CDCl₃ solutions referenced to TMS or the solvent residual peak unless otherwise indicated. IR spectra were taken as neat for liquids on NaCl plates or as KBr pellets for solids using a Perkin-Elmer 1600 FTIR spectrometer. High Resolution Mass Spectra were obtained on a JOEL JMS-HX110 HF mass spectrometer. Flash chromatography was performed on SILICYCLE silica gel (230-400 mesh). All chemicals were purchased from Aldrich and used as received. All reactions were monitored by Thin Layer Chromatography.

Ultraviolet spectra were measured on a Cary 100 UV-Visible spectrophotometer and recorded in EtOH solutions. Recorded λₘₐₓ is that of the longest wavelength transition. Fluorescence measurements were taken on a Jobin Yvon Fluorolog fluorescence spectrofluorometer in potassium phosphate pH 7.0 buffer unless otherwise indicated. Quantum yields were measured relative to 9,10 diphenylanthracene in EtOH (Heinrich, G.; Schoof, S.; Gusten, H. J. Photochem. 1974/75, 3, 312-320) for probes 1-4 and alcohols 8, 11, 13, and 15, or Coumarin 6 in EtOH (Reynolds, G. A.; Drexhage, K. H. Opt. Commun. 1975, 13, 222) for probes 5-7 and alcohols 19, 21, and 22. Reported quantum efficiencies are the average of at least three independent preparations of the probes and their cognate alcohols.

Synthesis of Probes 1-7 and the Corresponding Alcohols

Synthesis of Probe 1

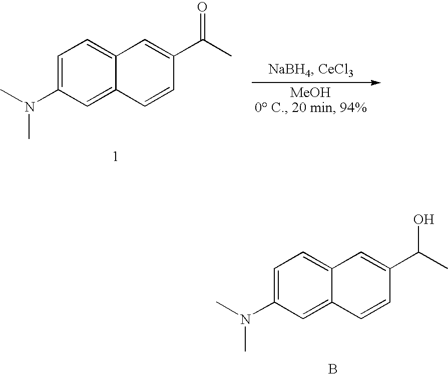

### 1-(6-Dimethylamino-naphthalen-2-yl)-ethanone (1)

This compound was prepared by a literature procedure and spectral data are consistent with those previously published (Jacobson, A.; Petric, A.; Hogenkamp, D.; Sinur, A.; Barrio, J. R. J. Am. Chem. Soc. 1996, 118, 5572-55790).

### 1-(6-Dimethylamino-naphthalen-2-yl)-ethanol (8)

CeCl₃.7H₂O (116 mg, 0.31 mmol) was added to a solution of 1 (50 mg, 0.23 mmol) in MeOH (10 ml) at 0° C., followed by addition of NaBH₄ (46 mg, 1.22 mmol). After 20 minutes, the reaction was quenched with a saturated aqueous solution of NH₄Cl and extracted with CHCl₃. Organic layer was dried over MgSO₄, evaporated and the crude product was purified by column chromatography on silica gel (CH₂Cl₂-EtOAc 98:2) to provide pure alcohol (47 mg, 94%).

NMR ¹H (300 MHz, CDCl₃) δ ppm:

7.67 (d, 1H, J1=9.0 Hz); 7.63 (bs, 1H); 7.63 (d, 1H, J1=8.5 Hz); 7.37 (dd, 1H, J1=8.5 Hz, J2=1.7 Hz); 7.15 (dd, 1H, J1=9.0 Hz, J2=2.5 Hz); 6.90 (d, 1H, J1=2.5 Hz); 4.99 (m, 1H); 3.03 (s, 6H); 1.79 (d, 1H, J1=3.5 Hz), 1.59 (d, 3H, J1=6.4 Hz).

NMR ¹³C (300 MHz, CDCl₃) δ ppm:

148.7; 139.3; 134.5; 128.7; 126.6; 126.5; 124.2; 123.6; 116.7; 106.5; 70.6; 40.9; 24.9.

IR (NaCl, cm⁻¹): 3358, 2969, 2875, 1632, 1606, 1507, 1444, 1382, 1334, 1171, 1069, 968, 845, 804, 676.

HRMS (FAB): 215.1308 (C₁₄H₁₇ON, M; calc 215.1310).

UV (EtOH): λₘₐₓ=348 nm.

Fluorescence (potassium phosphate pH 7.0): λₑₘ=429 nm, Φ_{f}=0.07.

Synthesis of Probe 2

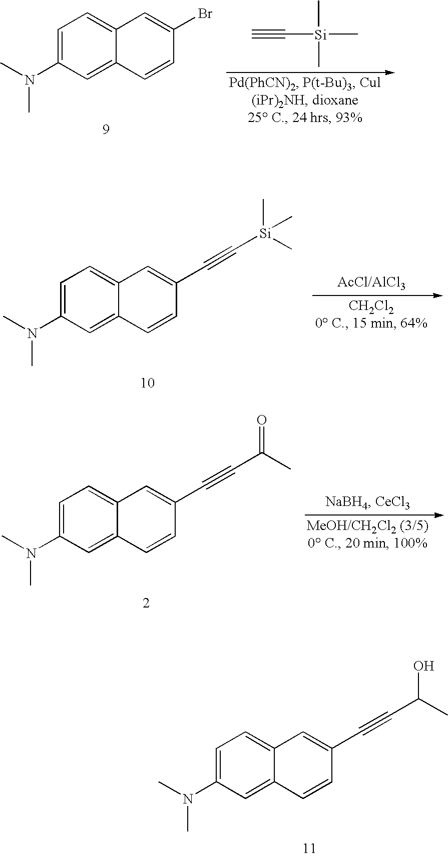

### Dimethyl-(6-trimethylsilanylethynyl-naphthalen-2-yl)-amine (10)

This compound was prepared by the procedure of Buchwald and Fu (Hundertmark, T.; Littke, A. F.; Buchwald, S. L.; Fu, G. C. Org. Lett. 2000, 2, 1729-1731) from bromide 9, which was obtained from 2-bromo-6-naphthol according to literature (Balo, C.; Fernandez, F.; Garcia-Mera, X.; Lopez, C. Org. Prep. Proced. Int. 2000, 32, 367-372). Pd(PhCN)₂Cl₂ (4.6 mg, 0.012 mmol), CuI (1.5 mg, 0.008 mmol), 9 (100 mg, 0.400 mmol), dioxane (1 ml), diisopropylamine (68 μl, 0.024 mmol) and (trimethylsilyl)acetylene (110 μl, 0.800 mmol) were mixed in a vial under argon and allow to stir 24 hrs at room temperature. The resultant mixture was diluted with EtOAc, washed with brine and dried over MgSO₄. Following solvent evaporation and product purification by column chromatography using silica gel and hexanes-EtOAc 98:2, 10 was yielded (99 mg, 93%).

NMR ¹H (300 MHz, CDCl₃) δ ppm:

7.81 (bs, 1H); 7.61 (d, 1H. J1=9.1 Hz); 7.52 (d, 1H, J1=8.5 Hz); 7.36 (dd, 1H, J1=8.5 Hz, J2=1.6 Hz); 7.11 (dd, 1H, J1=9.1 Hz, J2=2.5 Hz); 6.83 (d, 1H, J1=2.5 Hz); 3.05 (s, 6H); 0.27 (s, 9H).

NMR ¹³C (300 MHz, acetone-d) δ ppm:

150.4; 135.8; 132.3; 129.5; 129.3; 127.0; 126.8; 117.6; 116.5; 107.4; 106.5; 92.9; 40.6; 0.1.

IR (NaCl, cm⁻¹): 2960, 2901, 2812, 2147, 1629, 1598, 1247, 894, 850, 838, 809.

HRMS (FAB): 267.1442 (C₁₇H₂₁NSi, M; calc 267.1443).

### 4-(6-Dimethylamino-naphthalen-2-yl)-but-3-yn-2-one (2)

AcCl (13 μl, 0.18 mmol) was added to a solution of 10 (43 mg, 0.16 mmol) in CH₂Cl₂ (2 ml) at 0° C., followed by addition of AlCl₃ (107 mg, 0.80 mmol). After 15 minutes, the reaction was quenched with H₂O and extracted with EtOAc. After the organic layer was dried over MgSO₄, the solvent was removed, and the residue was purified by column chromatography on silica gel (hexanes-EtOAc 98:2) to yield ketone 2 (55 mg, 64%).

NMR ¹H (300 MHz, CDCl₃) δ ppm:

7.96 (bs, 1H); 7.66 (d, 1H, J1=9.1 Hz); 7.56 (d, 1H, J=8.5 Hz); 7.41 (dd, 1H, J1=8.5 Hz, J2=1.6 Hz); 7.14 (dd, 1H, J1=9.1 Hz, J2=2.5 Hz); 6.82 (d, 1H, J1=2.5 Hz); 3.09 (s, 6H); 2.46 (s, 3H).

NMR ¹³C (300 MHz, CDCl₃) δ ppm:

1184.6; 149.8; 135.9; 134.6; 129.3; 129.1; 126.3; 125.5; 116.5; 111.9; 105.4; 93.1; 88.5; 40.4; 32.7.

IR (NaCl, cm⁻¹): 2892, 2817, 2180, 1667, 1625, 1507, 1354, 1280, 1190, 1168, 896, 851, 810.

HRMS (FAB): 237.1138 (C₁₆H₁₅ON, M; calc 237.1154).

UV (EtOH): λₘₐₓ=389 nm.

Fluorescence (potassium phosphate pH 7.0): 448 nm, Φ_{f}=0.00.

### 4-(6-Dimethylamino-naphthalen-2-yl)-but-3-yn-2-ol (11)

Reduction of 2 (20 mg, 0.084 mmol) in MeOH—CH₂Cl₂ 3:5 (5 ml) followed a procedure analogous to that used for the preparation of 8. Column chromatography on silica gel (CH₂Cl₂) afforded alcohol 11 (20 mg, 100%).

NMR ¹H (300 MHz, CDCl₃) δ ppm:

7.77 (bs, 1H); 7.61 (d, 1H, J1=9.1 Hz); 7.54 (d, 1H, J1=8.5 Hz); 7.33 (dd, 1H, J1=8.5 Hz, J2=1.5 Hz); 7.12 (dd, 1H; J1=9.1 Hz, J2=2.4 Hz); 6.83 (d, 1H, J1=2.4 Hz); 4.78 (m, 1H); 3.05 (s, 6H); 1.88 (d, 1H, J1=4.8 Hz); 1.57 (d, 3H, J1=6.5 Hz).

NMR ¹³C (300 MHz, CDCl₃) δ ppm:

149.1; 134.5; 131.4; 128.8; 128.7; 126.1; 126.0; 116.6; 115.3; 105.9; 89.9; 85.0; 59.0; 40.6; 24.5.

IR (NaCl, cm⁻¹): 3346, 2982, 2930, 2882, 1628, 1598, 1505, 1389, 1101, 1072, 1035, 893, 848, 809.

HRMS (FAB): 239.1305 (C₁₆H₁₇ON, M; calc 239.1310).

UV (EtOH): λₘₐₓ=361 nm.

Fluorescence (potassium phosphate pH 7.0): 440 nm, Φ_{f}=0.08.

Synthesis of Probe 3

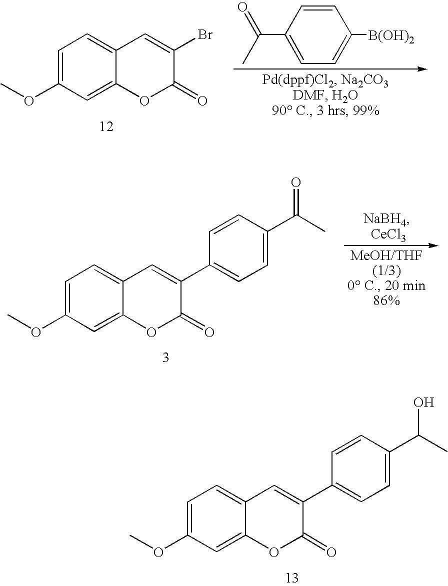

### 3-(4-Acetyl-phenyl)-7-methoxy-coumarin (3)

Bromide 12 (400 mg, 1.57 mmol), obtained by bromination of 7-methoxycoumarin, was mixed with 4-acetylphenylboronic acid (283 mg, 1.72 mmol), PdCl₂dppf (40 mg, 0.047 mmol), Na₂CO₃ (831 mg, 7.84 mmol), H₂O (3.92 ml) and DMF (16 ml) under argon. The resulting mixture was heated to 90° C. and stirred until completion (3 hrs) The cooled mixture was then diluted with water and extracted with CH₂Cl₂. Combined organic fractions were dried over MgSO₄. Following the evaporation of solvent, the residue was purified by column chromatography on silica gel (CH₂Cl₂) to afford desired product 3 (456 mg, 99%).

NMR ¹H (300 MHz, CDCl₃) δ ppm:

8.00 (m, 2H); 7.83 (m, 2H); 7.77 (bs, 1H); 7.46 (d, 1H, J1=8.4 Hz); 6.88 (m, 2H); 3.90 (s, 3H); 2.64 (s, 3H).

NMR ¹³C (300 MHz, CDCl₃) δ ppm:

197.6; 163.1; 160.5; 155.6; 141.0; 139.6; 136.6; 129.2; 128.5; 128.4; 123.5; 113.1; 113.1; 100.4; 55.9; 26.7.

IR (NaCl, cm⁻¹): 3070, 2962, 1710, 1670, 1613, 1505, 1442, 1360, 1275, 1198, 1122, 1022, 929, 859, 829, 776.

HRMS (FAB): 295.0967 (C₁₈H₁₅O₄, M+1; calc 295.0970).

UV (EtOH): λₘₐₓ=348 nm.

Fluorescence (potassium phosphate pH 7.0): 462 nm, Φ_{f}=0.00.

### 3-[4-(1-Hydroxy-ethyl)-phenyl]-7-methoxy-coumarin (13)

Reduction of 3 (42 mg, 0.14 mmol) in MeOH-THF 1:3 (15 ml) proceeded as described for the preparation of 8. Column chromatography on silica gel (eluent gradient: CH₂Cl₂ to CH₂Cl₂-EtOAc 8:2) afforded alcohol 13 (36 mg, 86%).

NMR ¹H (300 MHz, CDCl₃) δ ppm:

7.75 (s, 1H); 7.67 (m, 2H); 7.44 (m, 3H); 4.95 (m, 1H); 3.89 (s, 3H); 1.85. (d, 1H, J1=3.4 Hz); 1.52 (d, 3H, J1=6.4 Hz).

NMR ¹³C (300 MHz, CDCl₃) δ ppm:

162.5; 160.9; 155.2; 146.1; 139.8; 134.0; 128.8; 128.4; 125.4:; 124.4; 113.3; 112.7; 100.3; 70.0; 55.7; 25.1.

IR (NaCl, cm⁻¹): 3415, 2971, 1719, 1611, 1057, 1443, 1364, 1271, 1202, 1163, 1120, 1089, 1026, 832.

HRMS (FAB): 297.1112 (C₁₈H₁₇O₄, M+1; calc 297.1127).

UV (EtOH): λₘₐₓ=342 nm.

Fluorescence (potassium phosphate pH 7.0): 429 nm, Φ_{f}=0.12.

Synthesis of Probe 4

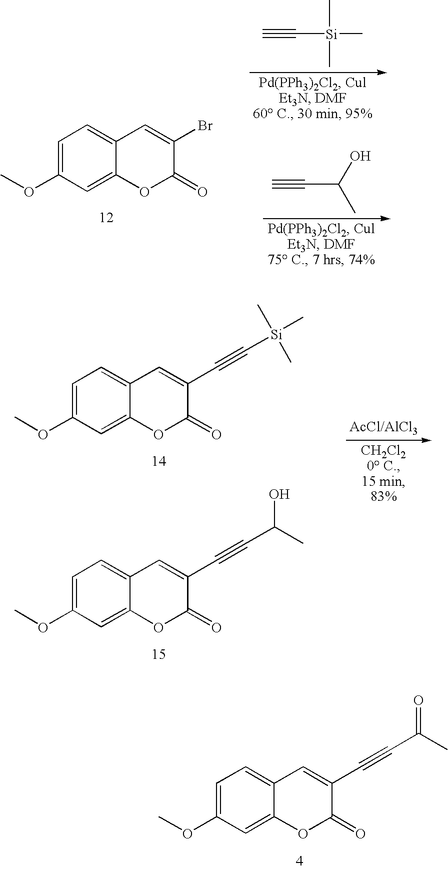

### 7-Methoxy-3-trimethylsilanylethynyl-coumarin (14)

PdCl₂(PPh₃)₂ (28 mg, 0.04 mmol), CuI (8 mg, 0.04 mmol), Et₃N (278 μl, 2.00 mmol) and (trimethylsilyl)acetylene (138 μl, 1.50 mmol) were added to a solution of bromide 12 (255 mg, 1.00 mmol) in dry DMF (10 ml) under argon. The resulting solution was heated to 60° C. and allowed to react 30 minutes. The mixture was then cooled, diluted with water, and extracted with CH₂Cl₂. The organic fractions were then combined and dried over MgSO₄. Removal of solvent in vacuo and purification of the residue by column chromatography on silica gel (CH₂Cl₂) afforded product 14 (259 mg, 95%).

NMR ¹H (300 MHz, CDCl₃) δ ppm:

7.82 (s, 1H); 7.32 (d, 1H, J1=8.6 Hz); 6.83 (dd, 1H, J1=8.6 Hz, J2=2.4 Hz); 6.78 (d, 1H, J1=2.4 Hz); 3.86 (s, 3H); 0.26 (s, 9H).

NMR ¹³C (300 MHz, acetone-d) δ ppm:

164.6; 159.5; 156.5; 147.5; 130.4; 113.8; 113.2; 109.3; 101.3; 100.2; 99.8; 56.5; −0.2.

IR (NaCl, cm⁻¹): 3040, 2961, 2840, 1721, 1600, 1441, 1368, 1272, 1247, 1034, 973, 831, 807, 765.

HRMS (FAB): 272.0869 (C₁₅H₁₆O₃Si, M; calc 272.0869).

### 7-Methoxy-3-(3-oxo-but-1-ynyl)-coumarin (4)

Compound 14 (103 mg, 0.38 mmol) was converted into ketone 4 by the procedure used for the preparation of 2. Column chromatography of the crude product on silica gel (CH₂Cl₂) provided 4 (76 mg, 83%).

NMR ¹H (300 MHz, CDCl₃) δ ppm:

8.00 (bs, 1H); 7.40 (d, 1H, J1=8.7 Hz); 6.88 (dd, 1H, J1=8.7 Hz, J2=2.3 Hz); 6.81 (d, 1H, J1=2.3 Hz); 3.90 (s, 3H); 2.47 (s, 3H).

NMR ¹³C (300 MHz, CDCl₃) δ ppm:

184.1, 164.7; 158.9; 156.3; 149.8; 129.7; 113.8; 112.1; 106.0; 100.9; 92.2; 84.1; 56.0; 32.6.

IR (NaCl, cm⁻¹): 3046, 2197, 1725, 1664, 1617, 1596, 1557, 1504, 1368, 1273, 1250, 1152, 1116, 1019, 836.

HRMS (FAB): 242.0572 (C₁₄H₁₀O₄, M+1; calc. 242.0579).

UV (EtOH): λₘₐₓ=368 nm.

Fluorescence (potassium phosphate pH 7.0): 416 nm, Φ_{f}=0.00.

### 3-(3-Hydroxy-but-1-ynyl)-7-methoxy-coumarin (15)

Alcohol 15 was prepared by Sonogashira coupling of bromide 12 (100 mg, 0.39 mmol) and but-3-yn-2-ol (32 μl, 0.43 mmol) under conditions similar to that used for the preparation of 14. After 7 hours at 75° C., the reaction was complete. The crude alcohol was purified by column chromatography on silica gel (CH₂Cl₂-EtOAc 95:5) to afford product 15 (96 mg, 74%).

NMR ¹H (300 MHz, CDCl₃) δ ppm:

7.81 (bs, 1H); 7.35 (d, 1H, J1=8.6 Hz); 6.86 (dd, 1H, J1=8.6 Hz, J2=2.4 Hz); 6.81 (d, 1H, J1=2.4 Hz); 4.79 (m, 1H); 3.88 (s, 3H); 2.26 (d, 1H, J1=5.2 Hz); 1.56 (d, 3H, J1=6.6 Hz).

NMR ¹³C (300 MHz, CDCl₃) δ ppm:

163.3; 160.1; 155.2; 145.5; 128.8; 113.2; 112.4; 108.6; 100.7; 96.7; 77.9; 58.7; 55.8; 24.0.

IR (NaCl, cm⁻¹): 3414, 2983, 2939, 2843, 1733, 1618, 1506, 1365, 1269, 1121, 1024, 768.

HRMS (FAB): 244.0744 (C₁₄H₁₂O₄, M; calc 244.0736).

UV (EtOH): λₘₐₓ=346 nm.

Fluorescence (potassium phosphate pH 7.0): 420 nm, Φ_{f}=0.18.

Synthesis of Probe 5

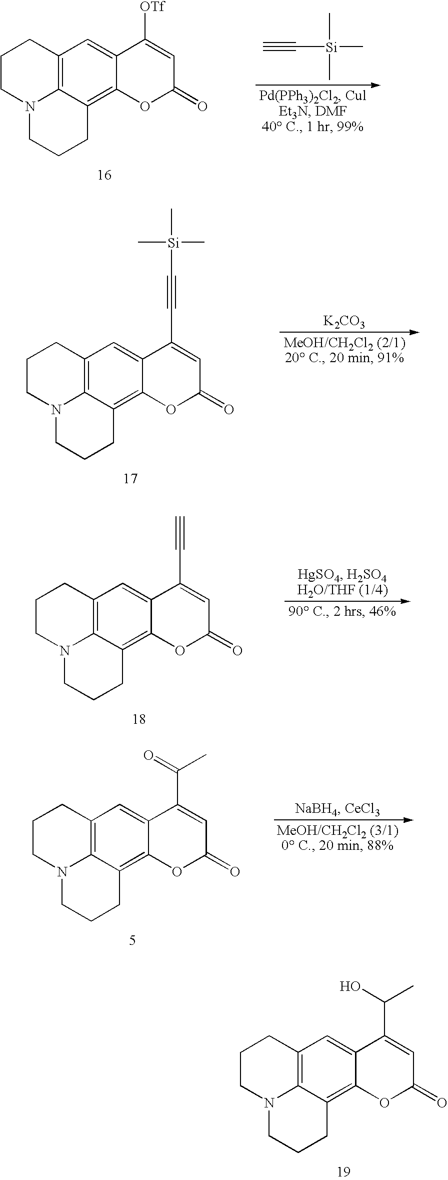

### 8-Trimethylsilanylethynyl-2,3,5,6-tetrahydro-1H,4H-11-oxa-3a-aza-benzo[de]anthracen-10-one (17)

Triflate 16 (707 mg, 1.82 mmol), obtained from 8-hydroxyjulolidine according to the literature (Coleman, R. S.; Madaras, M. L. J. Org. Chem. 1998, 63, 5700-5703), was coupled with (trimethylsilyl)acetylene (377 μl, 2.72 mmol) under conditions described for the preparation of 14. The reaction was complete after 1 hr at 40° C. Column chromatography on silica gel (CH₂Cl₂) provided desired product 17 (607 mg, 99%).

NMR ¹H (300 MHz, CDCl₃) δ ppm:

7.16 (s, 1H); 6.11 (s, 1H); 3.26 (m, 4H); 2.83 (m, 4H); 1.97 (m, 4H); 0.31 (s, 9H).

NMR ¹³C (300 MHz, CDCl₃) δ ppm:

161.8; 151.1; 146.1; 137.0; 123.5; 118.3; 110.8; 107.6; 106.6; 106.3; 98.8; 49.9; 49.4; 27.6; 21.4; 20.4; 20.2; −0.4.

IR (NaCl, cm⁻¹): 2946, 2848, 1701, 1612, 1546, 1511, 1421, 1367, 1310, 1245, 1184, 843.

HRMS (FAB): 338.1574 (C₂₀H₂₄O₂NSi, M+1; calc 338.1576).

### 8-Ethynyl-2,3,5,6-tetrahydro-1H,4H-11-oxa-3a-aza-benzo[de]anthracen-10-one (18)

Powdered K₂CO₃ (600 mg) was added to a solution of 17 (580 mg, 1.72 mmol) in MeOH—CH₂Cl₂ 2:1 (30 ml). The mixture was stirred at room temperature until the reaction was complete (20 min). Reaction mixture was diluted with CHCl₃, filtered, and washed with brine. The resultant organic layers were combined and dried over MgSO₄, after which the solvent was removed in vacuo. Purification by column chromatography on silica gel (eluent gradient: CH₂Cl₂ to CH₂Cl₂-EtOAc 95:5) afforded terminal alkyne 18 (416 mg, 91%).

NMR ¹H (300 MHz, CDCl₃) δ ppm:

7.19 (s, 1H); 6.16 (s, 1H); 3.58 (s, 1H); 3.27 (m, 4H); 2.87 (m, 2H); 2.78 (m, 2H); 1.97 (m, 4H).

NMR ¹³C (300 MHz, CDCl₃) δ ppm:

161.6; 151.1; 146.3.; 136.3; 123.4; 118.5; 111.7; 107.6; 106.4; 87.5; 78.0; 49.9; 49.5; 27.5; 21.3; 20.4; 20.2.

IR (NaCl, cm⁻¹): 3221, 2931, 2838, 2103, 1699, 1616, 1519, 1428, 1371, 1311, 1176, 826.

HRMS (FAB): 266.1193 (C₁₇H₁₆O₂N, M+1; calc 266.1181).

### 8-Acetyl-2,3,5,6-tetrahydro-1H,4H-11-oxa-3a-aza-benzo[de]anthracen-10-one (5)

HgSO₄ (112 mg, 0.38 mmol) was added to a solution of 18 (100 mg, 0.38 mmol) in THF (8 ml), followed by addition of conc. H₂SO₄ (105 μl, 1.88 mmol) in H₂O (2 ml). The reaction mixture was heated in a sealed tube at 90° C. for 2 hrs. After cooling to room temperature, a spatula tip of NaHCO₃ was added and the mixture was evaporated to dryness. MgSO₄ was added and the residual solids were washed thoroughly with CHCl₃. The solvent was the evaporated and the residue purified by column chromatography on silica gel (CH₂Cl₂-Et₂O 95:5) yielding ketone 5 (49 mg, 46%).

NMR ¹H (300 MHz, CDCl₃) δ ppm:

7.18 (s, 1H); 6.13 (s, 1H); 3.27 (m, 4H); 2.88 (m, 2H); 2.74 (m, 2H); 2.55 (s, 3H); 1.96 (m, 4H).

NMR ¹³C (300 MHz, CDCl₃) δ ppm:

200.4; 162.1; 152.1; 150.8; 146.2; 123.2; 118.7; 106.8; 106.8; 103.7; 49.9; 49.4; 29.7; 27.6; 21.3; 20.4; 20.3.

IR (NaCl, cm⁻¹): 2933, 2844, 1694, 1611, 1544, 1525, 1434, 1373, 1352, 1311, 1232, 1170, 1148.

HRMS (FAB): 283.1195 (C₁₇H₁₇O₃N, M; calc 283.1208).

UV (EtOH): λₘₐₓ=418 nm.

Fluorescence (potassium phosphate pH 7.0): 520 nm, Φ_{f}=0.00.

### 8-(1-Hydroxy-ethyl)-2,3,5,6-tetrahydro-1H,4H-11-oxa-3a-aza-benzo[de]anthracen-10-one (19)

Reduction of 5 (16 mg, 0.056 mmol) in MeOH—CH₂Cl₂ 3:1 (5 ml) proceeded by previously described procedures (used for preparation of 8). Column chromatography on silica gel (eluent gradient: CH₂Cl₂ to CH₂Cl₂-EtOAc 9:1) afforded alcohol 19 (14 mg, 88%).

NMR ¹H (300 MHz, CDCl₃) δ ppm:

7.01 (s, 1H); 6.24 (s, 1H); 5.14 (m, 1H); 3.26 (m, 4H); 2.87 (m, 2H); 2.77 (m, 2H); 2.07 (d, 1H, J1=3.8 Hz); 2.10 (m, 4H); 1.55 (d, 3H, J1=6.5 Hz).

NMR ¹³C (300 MHz, CDCl₃) δ ppm:

163.0; 159.4; 151.4; 145.6; 121.0; 118.0; 107.1; 105.9; 103.8; 65.9; 49.9; 49.5; 27.8; 23.6; 21.5; 20.6; 20.5.

IR (NaCl, cm⁻¹): 3396, 2936, 2843, 1688, 1611, 1554, 1520, 1433, 1372, 1311, 1183, 1133.

HRMS (FAB) 286.1437 (C₁₇H₂₀O₃N, M+1; calc 286.1443).

UV (EtOH): λₘₐₓ=398 nm.

Fluorescence (potassium phosphate pH 7.0): 509 nm, Φ_{f}=0.21.

Synthesis of Probe 6

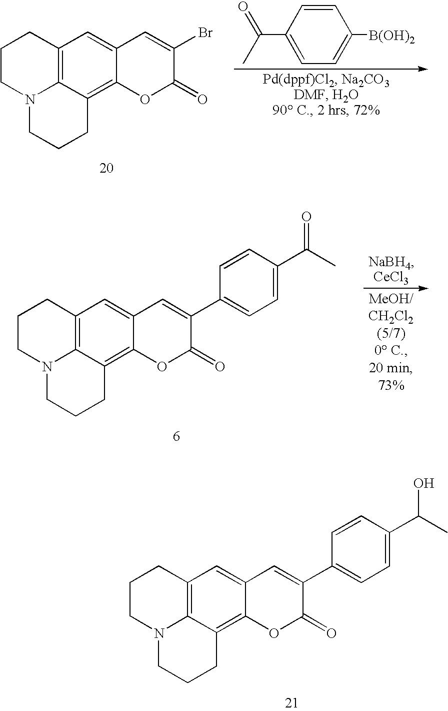

### 9-(4-Acetyl-phenyl)-2,3,5,6-tetrahydro-1H,4H-11-oxa-3a-aza-benzo[de]anthracen-10-one (6)

Bromide 20 (100 mg, 0.31 mmol), obtained by bromination of coumarin 6H, was coupled with 4-acetylphenylboronic acid (77 mg, 0.46 mmol), under similar conditions as those used for preparation of 3. Reaction was complete after 2 hrs at 90° C. Column chromatography on silica gel (eluent gradient: CH₂Cl₂ to CH₂Cl₂-EtOAc 95:5) provided desired ketone 6 (81 mg, 72%).

NMR ¹H (300 MHz, CDCl₃) δ ppm:

7.96 (m, 2H); 7.81 (m, 2H); 7.68 (s, 1H); 6.91 (s, 1H); 3.29 (m, 4H); 2.93 (m, 2H); 2.77 (m, 2H); 2.61 (s, 3H); 1.98 (m, 4H).

NMR ¹³C (300 MHz, CDCl₃) δ ppm:

197.7; 161.4; 151.5; 146.3; 141.7; 141.0; 135.6; 128.3; 128.0; 125.4; 118.7; 118.0; 108.7; 106.1; 50.0; 49.6; 27.4; 26.6; 21.4; 20.4; 20.2.

IR (NaCl, cm⁻¹): 2941, 2845, 1699, 1677, 1616, 1594, 1563, 1518, 1360, 1306, 1269, 1213, 1171.

HRMS (FAB): 359.1527 (C₂₃H₂₁O₃N, M; calc 359.1521).

UV (EtOH): λₘₐₓ=435 nm.

Fluorescence (potassium phosphate pH 7.0): 511 nm, Φ_{f}=0.01.

### 9-[4-(1-Hydroxy-ethyl)-phenyl]-2,3,5,6-tetrahydro-1H,4H-11-oxa-3a-aza-benzo[de]anthracen-10-one (21)

Reduction of 6 (15 mg, 0.041 mmol) in MeOH—CH₂Cl₂ 5:7 (6 ml) by the procedure used for preparation of 8 and recrystallization from CHCl₃-hexanes afforded alcohol 21 (11 mg, 73%).

NMR ¹H (300 MHz, CDCl₃) δ ppm:

7.66 (m, 2H); 7.58 (s, 1H); 7.40 (m, 2H); 6.88 (s, 1H); 4.92 (q, 1H, J1=6.4 Hz); 3.28 (m, 4H); 2.92 (m, 2H); 2.76 (m, 2H); 1.98 (m, 4H); 1.81 (bs, 1H); 1.51 (d, 3H, J1=6.4 Hz).

NMR ¹³C (300 MHz, CDCl₃) δ ppm:

161.9; 151.2; 145.8; 145.1; 140.8; 135.3; 128.3; 125.3; 125.1; 119.6; 118.5; 109.0; 106.3; 70.2; 50.0; 49.6; 27.5; 25.1; 21.5; 20.6; 20.3.

IR (NaCl, cm⁻¹): 3408, 2930, 2844, 1694, 1615, 1599, 1564, 1519, 1309, 1209, 1170, 839, 748.

HRMS (FAB): 361.1673 (C₂₃H₂₃O₃N, M; calc 361.1678).

UV (EtOH): λₘₐₓ=422 nm.

Fluorescence (potassium phosphate pH 7.0): 509 nm, Φ_{f}=0.14.

Synthesis of Probe 7

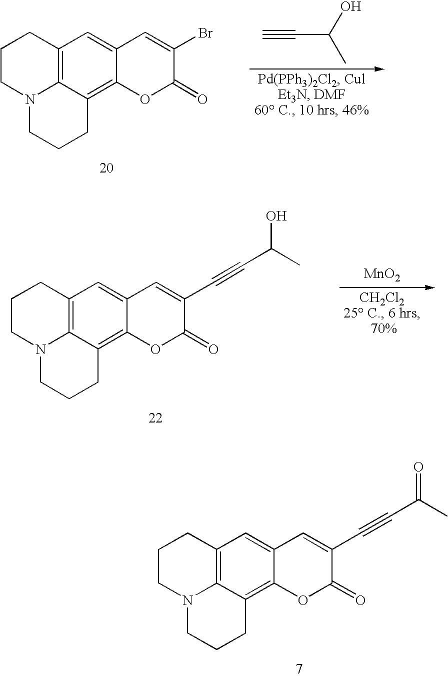

### 9-(3-Hydroxy-but-1-ynyl)-2,3,5,6-tetrahydro-1H,4H-11-oxa-3a-aza-benzo[de]anthracen-10-one (22)

Alcohol 22 was prepared by Sonogashira coupling of bromide 20 (100 mg, 0.31 mmol) and but-3-yn-2-ol (26 μl, 0.34 mmol) as described for the preparation of 14. The reaction was stopped after 10 hrs at 60° C. Column chromatography on silica gel (eluent gradient: CH₂Cl₂ to CH₂Cl₂-EtOAc 9:1) provided 22 (45 mg, 46%).

NMR ¹H (300 MHz, CDCl₃) δ ppm:

7.60 (s, 1H); 6.78 (s, 1H); 4.77 (m, 1H); 3.28 (m, 4H); 2.87 (m, 2H); 2.75 (m, 2H); 2.14 (d, 1H, J1=4.9 Hz); 1.97 (m, 4H); 1.54 (d, 3H, J1=6.6 Hz).

NMR ¹³C (300 MHz, CDCl₃) δ ppm:

161.6; 151.3; 146.6; 146.4; 125.0; 118.9; 108.1; 106.4; 102.8; 94.7; 79.3; 58.9; 50.1; 49.7; 27.4; 24.1; 21.3; 20.4; 20.2.

IR (NaCl, cm⁻¹): 3397, 2934, 2849, 1709, 1692, 1616, 1594, 1518, 1360, 1309, 1290, 1169, 765.

HRMS (FAB): 309.1365 (C₁₉H₁₉O₃N, M; calc 309.1365).

UV (EtOH): λₘₐₓ=429 nm.

Fluorescence (potassium phosphate pH 7.0): 508 nm, Φ_{f}=0.35.

### 9-(3-Oxo-but-1-ynyl)-2,3,5,6-tetrahydro-1H,4H-11-oxa-3a-aza-benzo[de]anthracen-10-one (7)

To alcohol 22 (30 mg, 0.097 mmol) dissolved in dry CH₂Cl₂ (3 ml) was added powdered MnO₂ (150 mg) at room temperature. The resulting suspension was stirred until the reaction was complete (6 hrs). The subsequent mixture was filtered through Celite, dried in vacuo, and purified by column chromatography on silica gel (eluent gradient: CH₂Cl₂ to CH₂Cl₂-EtOAc 98:2) to afford 7 (21 mg, 70%).

NMR ¹H (300 MHz, CDCl₃) δ ppm:

7.77 (s, 1H); 6.82 (s, 1H); 3.33 (m, 4H); 2.86 (m, 2H); 2.75 (m, 2H); 2.44 (s, 3H); 1.97 (m, 4H).

NMR ¹³C (300 MHz, CDCl₃) δ ppm:

184.3; 160.6; 152.3; 150.2; 148.2; 125.9; 119.4; 108.1; 106.2; 98.8; 92.4; 87.9; 50.2; 49.8; 32.5; 27.3; 21.0; 20.1; 20.0.

IR (NaCl, cm⁻¹): 2937, 2844, 2170, 1714, 1657, 1620, 1586, 1520, 1358, 1295, 1154, 760.

HRMS (FAB): 308.1295 (C₁₉H₁₈O₃N, M+1; calc 308.1287).

UV (EtOH): λₘₐₓ=464 nm.

Fluorescence (potassium phosphate pH 7.0): 512 nm, Φ_{f}=0.01.

Procedure for Enzymatic Screening of Selected Probes 1-7:

Horse Liver alcohol dehydrogenase (Lot Number 51K7520), Thermoanaerobium brockii NADP⁺ dependent alcohol dehydrogenase (Lot Number 033K4093), Pseudomonas testosteroni 3α-hydroxysteroid dehydrogenase (Lot Number 053K8624), and Bacillus sphaericus 12α-hydroxysteroid dehydrogenase (Lot Number 70K16621) were purchased from Sigma (St. Louis, Mo.). Yeast alcohol dehydrogenase (Lot Number 93122920), glycerol dehydrogenase (Lot Number 92110122), (D)-lactate dehydrogenase (Lot Number 92419236), (L)-lactate dehydrogenase (Lot Number 92801821), NAD⁺, NADP⁺, NADH, and NADPH were purchased from Roche. Enzyme activity was confirmed by compliance to supplier's quality control assays prior to usage. Rat and human 3α-hydroxysteroid dehydrogenases were provided by Professor Trevor Penning (University of Pennsylvania School of Medicine) and human amyloid-β peptide binding alcohol dehydrogenase was supplied by Professor Shi Du Yan (Columbia University School for Physicians and Surgeons).

Enzymatic assays were performed in triplicate on selected fluorogenic substrates according to the following protocol. To each well of a FALCON 96-well black flat bottom plate was added (1) 40 μL of 500 mM potassium phosphate buffer pH 7.0, (2) 113 μL of double deionized water, (3) 25 μL of 2 mM NADH (except for Pseudomonas testosteroni 3α-hydroxysteroid dehydrogenase, rat 3α-hydroxysteroid dehydrogenase, and Thermoanaerobium brockii NADP⁺ dependent alcohol dehydrogenase, in which cases 2 mM of NADPH was used), (4) 2 μL of a 3-5 mM solution of substrate in DMSO, and (5) 20 μL of a 40-50 μg/mL solution of enzyme. Reaction volumes were mixed thoroughly after addition of cofactor, substrate, and enzyme and allowed to react 12 hours at 25° C. Scanning of the 96-well plate was performed by the MicroMax 384 connected to a Jobin Yvon Fluorolog through F-3000 fiber optic cables.

Determination of Kinetic Parameters for AKR1C3

Fluorogenic substrate 5 reduction was monitored on a Hitachi F-4500 fluorimeter in Starna quartz cuvettes fluorometrically in 1 mL systems containing 100 mM potassium phosphate pH 6.0 containing excess of NADPH cofactor (250 μM) and various amounts of the substrate (0.1953-50 μM) dissolved in 4% acetonitrile. Aqueous assay components were added first, followed by addition of 20 μL of acetonitrile as a cosolvent, and then addition of 20 μL of the substrate in acetonitrile (total acetonitrile in the assay did not exceed 4%). Cuvettes were mixed thoroughly after addition of cofactor, cosolvent, and substrate. Reactions were initiated by the addition of 4 μL of dilute AKR1C3 (115 μg/mL) and were corrected for nonenzymatic rates. All reactions were followed by monitoring the increase in fluorescence of the product alcohol for 5 minutes at λₑₘ 510 nm with λₑₓ 440 nm (Excitation and emission band pass slits both at 2.5 nm, lamp 900 V) at 37° C. The initial velocities, expressed in units of nanomoles per minute, were calculated according to previously published procedures (Wierzchowski, J.; Dafeldecker, W. P.; Holmquist, B.; Vallee, B. L. Anal. Biochem. 1989, 178, 57-62): initial rate=[nₛₜ×(Fₜ−F₀)/(Fₛₜ)]/t where Fₜ and F₀ represent the fluorescence at time t and 0, nₛₜ is the nanomoles of the product standard, and Fₛₜ is the fluorescence resulting from nₛₜ of product. Kinetic constants were approximated using the GraFit (Erithacus Software, Surrey, UK) non-linear regression analysis program to fit the untransformed data to a hyperbolic function as originally described (Wilkinson, G. N. Biochem. J. 1961, 80, 324-332), yielding estimated values of k_{cat}, Kₘ, and their associated standard errors.

AKR1C3

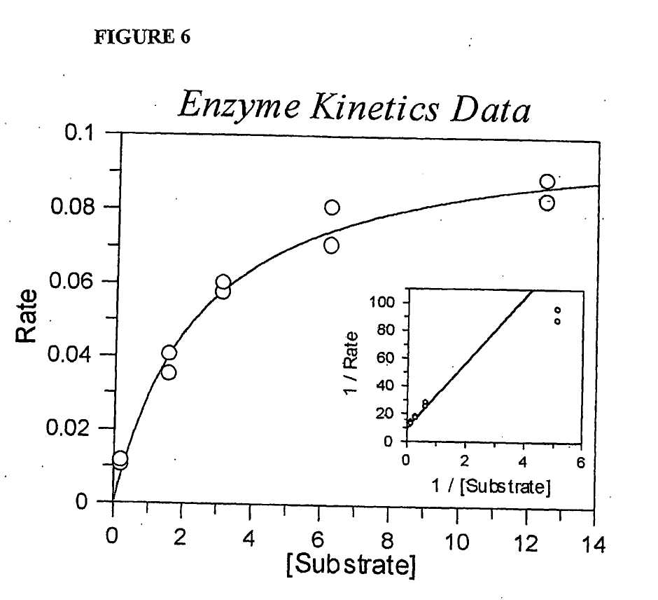

Enzyme kinetic data for this enzyme is shown in FIG. 6. The parameter values are shown in Table 2.

TABLE 2
AKRlC3 kinetic data.

| Parameter | Value | Std. Error |
|---|---|---|
| Vmax | 0.1039 | 0.0049 nmol/min |
| Km | 2.4637 | 0.3511 uM |
| kcat | 8.244 | 0.389 min⁻¹ |
| kcat/Km | 335 | min⁻¹/mM⁻¹ |
| Spec. activity | 0.226 | 0.011 umol/min/mg |

AKR1C3 kinetic data was also performed by HPLC separation of the fluorogenic substrate and its product alcohol and measurement of ketone to alcohol ratios. This data was found to correlate well with kinetic parameters determined fluorometrically (Yee, D. J.; Balsanek, V.; Sames, D. unpublished results).

Fluorescence Spectra of Probes 1-7

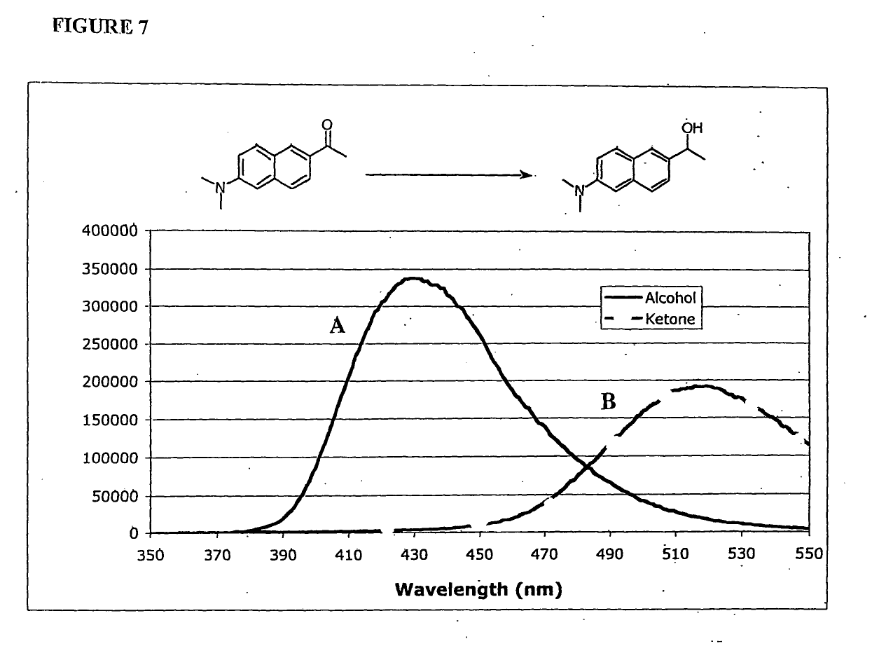

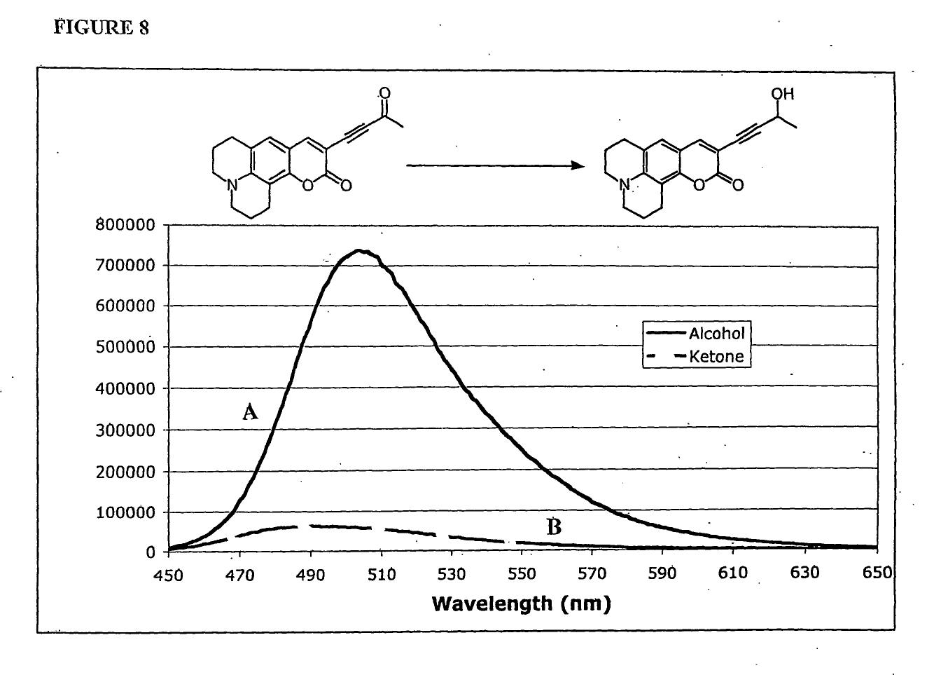

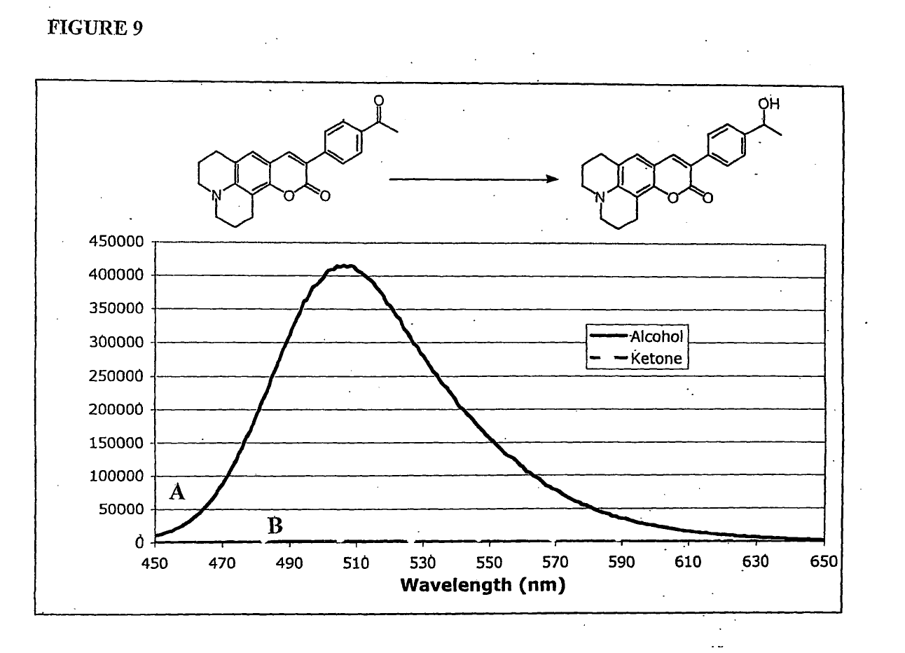

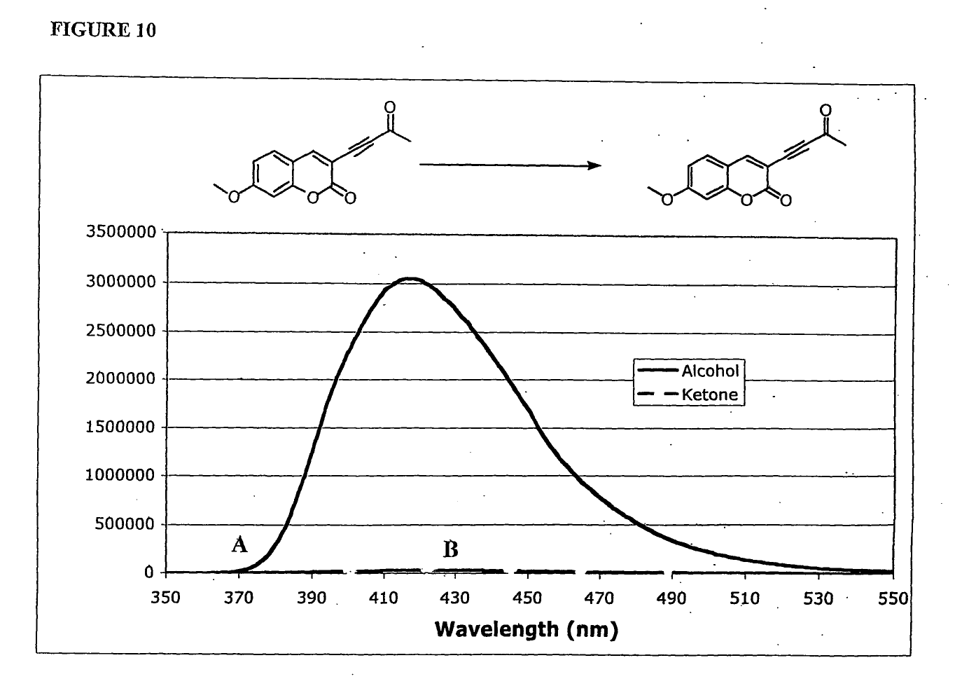

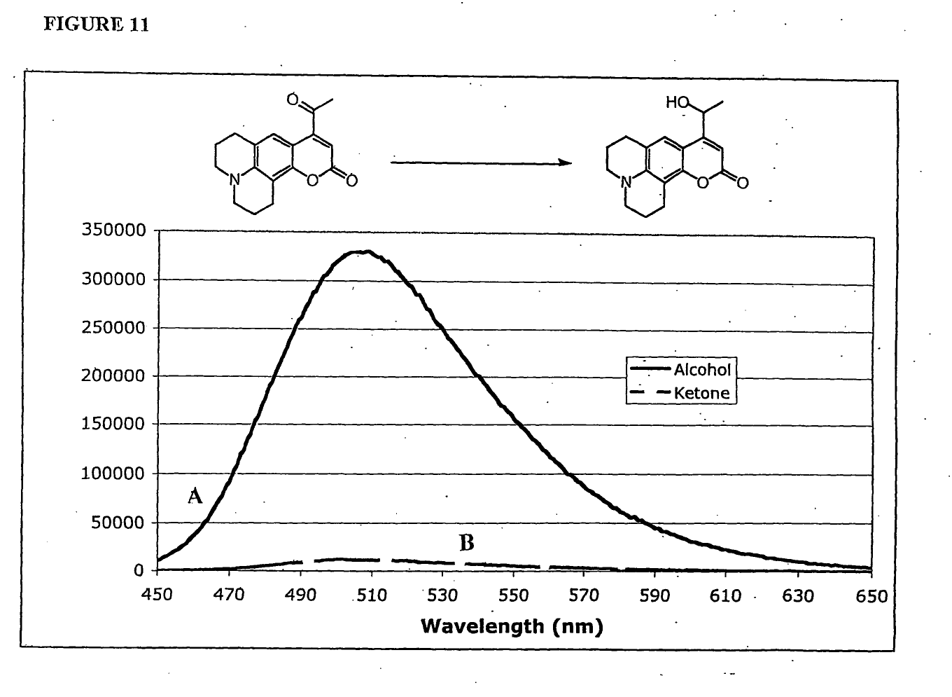

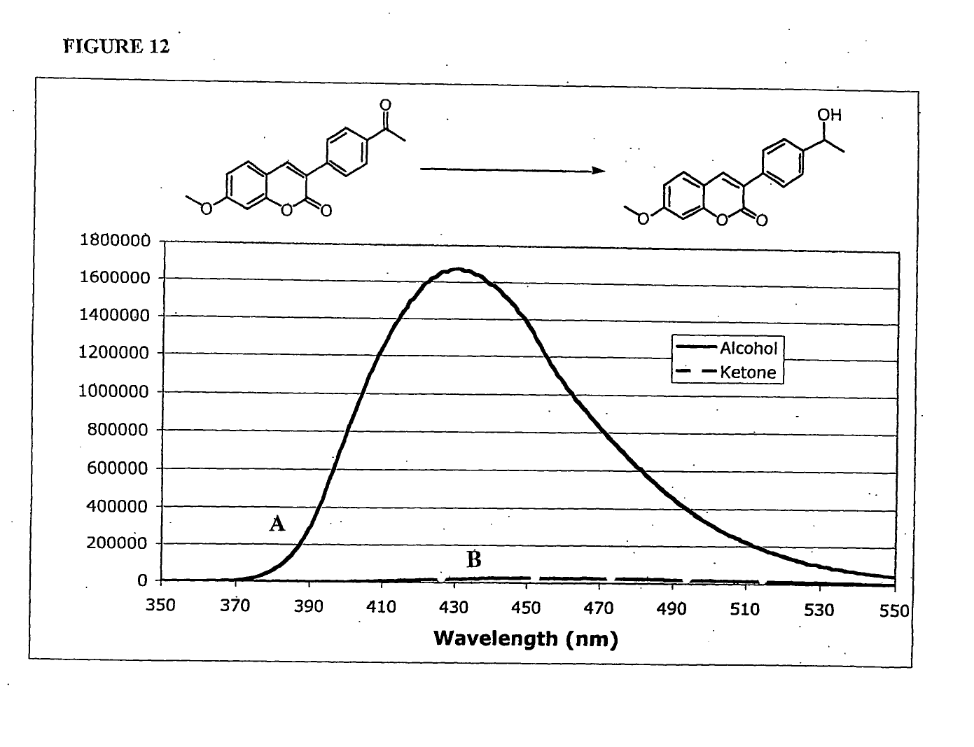

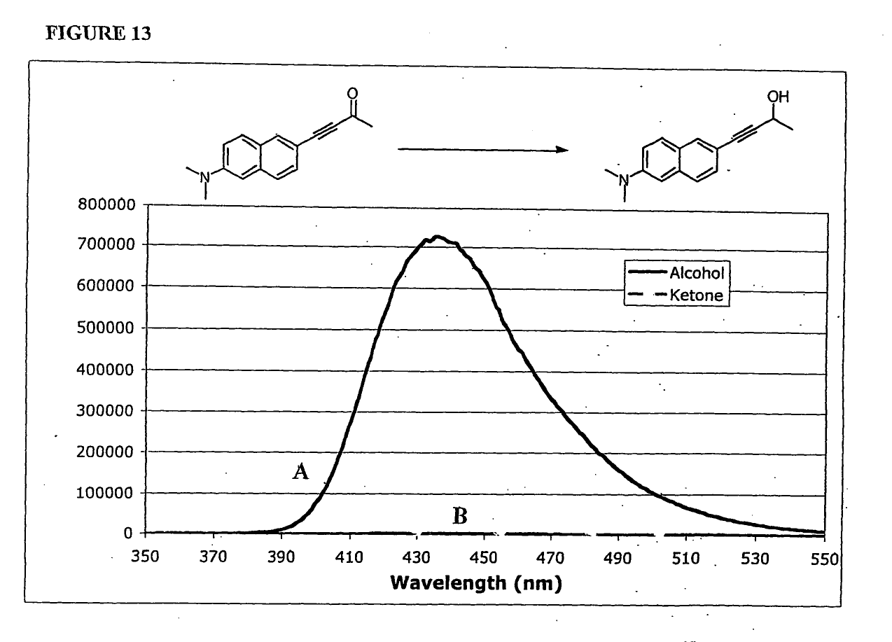

Compounds 1-4 were excited at 340 nm, while compounds 5-7 were excited at 440 nm. Fluorescence emission spectra were recorded with 10 μM solutions (<1% DMSO v/v) in 100 mM potassium phosphate buffer (pH 7.0). Spectra are shown in FIGS. 7-13 for probes 1-7.

Preparation and Testing of Derivatives of Probe 5:

A diverse array of fluorogenic probes for human hydroxysteroid dehydrogenases (HSDs) was synthesized and submitted to photophysical evaluation, followed by screening against a panel of oxidoreductases. This process identified compound 5 as a selective probe for 3α-HSDs. A subsequent structure-activity analysis of probe 5 resulted in the discovery of a second generation of fluorogenic probes, some of which proved selective for AKR isoforms. Namely, probes 5c, 5d, and 5h showed excellent selectivity for AKR1C3, while probe 5i demonstrated good preference for AKR1C2 (as judged by kinetic parameters k_{cat} and Kₘ). Most importantly, we found that phenyl ketone probe 5c was selective for AKR isoforms in lysates of human hepatoma cells HepG2. The activity of these specific enzymes could be measured optically in cellular extracts known to contain several hundred oxidoreductase enzymes.

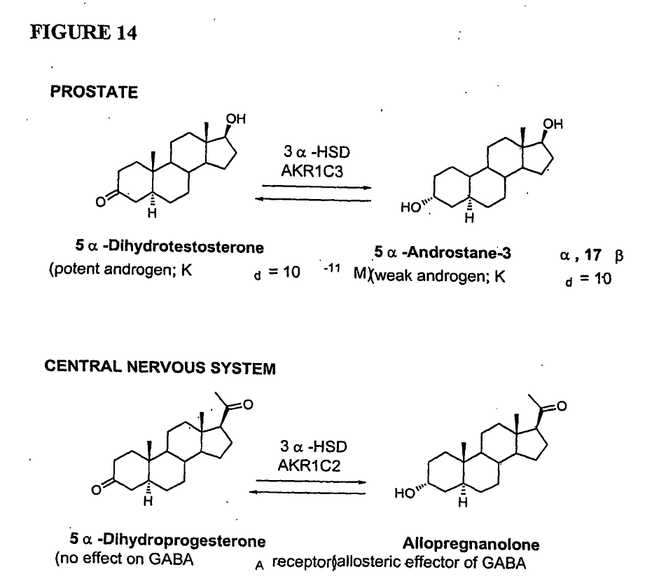

AKR1C3 contains high 17β-HSD activity and it is involved in the peripheral formation of androgens and estrogens, reactions that may be important in prostate and breast cancer (Penning, T. M.; Burczynski, M. E.; Jez, J. M.; Hung, C. F.; Lin, H. K.; Ma, H.; Moore, M.; Palackal, N.; Ratnam, K. Biochem J 2000, 351, 67-77), (see FIG. 14). Moreover, AKR1C3 also exhibits prostaglandin synthase activity (Komoto, J.; Yamada, T.; Watanabe, K.; Takusagawa, F. Biochemistry 2004, 43, 2188-2198). Although the assignment of precise metabolic functions to each human isozyme is ongoing, AKR1C2 and AKR1C3 are of particular interest. In fact, AKR1C2 levels were elevated in epithelial cells from prostate cancer; and this may contribute to the development of androgen independent tumors (Rizner, T. L.; Lin, H. K.; Peehl, D. M.; Steckelbroeck, S.; Bauman, D. R.; Penning, T. M. Endocrinology 2003, 144, 2922-2932). In addition, the structure-function relationship of 3α-hydroxysteroid dehydrogenases has been studied in both rat and human isoforms (e.g. see Penning et al., J. Steroid Biochem. And Mol. Biol. 85, 247-255 (2003)). These findings together with the proposed physiological functions of HSDs provide a strong impetus for the development of selective imaging probes for these enzymes.

Design and Synthesis of Probe 5 Analogs: With probe 5 in hand, we set the following goals for this study: (1) to elucidate, through chemical synthesis, the key structural features of 5 responsible for its activity and selectivity, (2) to explore the possibility of targeting individual HSD isozymes within the AKR family, and (3) to investigate the selectivity of the best candidates in human cellular extracts.

Mindful of these goals, the analysis of compound 5 suggested several points of structural variation, including the ketone R group, C-3 position, and the amine at C-7 position (FIG. 3). In particular, we were interested in the importance of the ketone substitution as well as the nitrogen-containing rings with regards to the activity and selectivity of these compounds as enzyme substrates.

Synthesis. All methylketone probes were prepared via two methods (Scheme 1). In Method A, which was used to prepare probe 5g and the original probe 5, the coumarin moiety was formed by condensation of phenol 8 with bis(2,4,6-trichlorophenyl) malonate in refluxing toluene (Knierzinger, A.; Wolfbeis, O. S. J. Heterocyclic Chem. 1980, 17, 225-261). The resulting 4-hydroxycoumarine was treated with Tf₂O, affording triflate 9, which was subjected to Sonogashira-Hagihara coupling with trimethyl-silylacetylene. After desilylation, terminal alkyne 10 was converted into the desired methylketone 5g using Hg(II)-mediated hydration. Compounds 5h and 5i were prepared directly from the corresponding phenols using Method B, Scheme 1. The von Pechmann condensation of the aminophenols with methyl

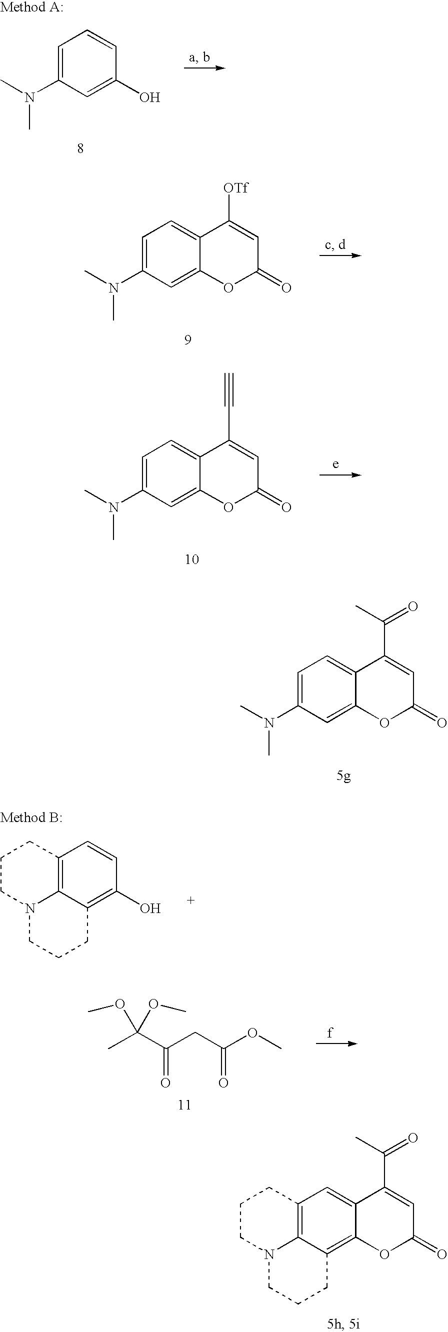

4,4-dimethoxy-3-oxovalerate 11 was accomplished by using InCl₃ (Bose, D. S.; Rudradas, A. P.; Babu, M. H. Tetrahedron Letters 2002, 43, 9195-9197) as a reagent to give the methylketones in moderate yields (25-35%). Employment of traditional reagents such as ZnCl₂ (Sethna, S.; Phadke, R. Org. React. 1953, 7, 1-58) resulted in lower yields (10%), while acidic catalysts (e.g. H₂SO₄) were virtually ineffective.

Scheme 1. Synthesis of methylketone probes^{a}

^{a}(a) Bis(2,4,6-trichlorophenyl) malonate, PhMe, reflux, 85%; (b) Tf₂O, Et₃N, CH₂Cl₂, −15° C., 60%; (c) Trimethylsilylacetylene, PdCl₂ (PPh₃)₂, CuI, Et₃N, DMF, 60° C., 90%; (d) K₂CO₃, MeOH/CH₂Cl₂, RT, 97%; (e) H₂O, HgSO₄, H₂SO₄, THF, 90° C.; 50-95% (f) InCl₃, MeOH, 75° C. , 25-35%.

Two different methods were also used to prepare 4-acylanalogues of probe 5 (Scheme 2). Method C involved Stille coupling of triflate 12 with tributylvinyltin. Resulting 4-vinylcoumarin 13, formed in a nearly quantitative yield, was converted to aldehyde 14 by dihydroxylation of the vinyl group using catalytic dihydroxylation protocol (OsO₄/NMO), followed by Pb(OAc)₄ oxidation of the vicinal diol. Addition of Grignard reagents to aldehyde 14 resulted in the formation of the desired secondary alcohols in moderate yields (40-50%), accompanied by a significant amount of reduction of the aldehyde (20%). After separation, the alcohols were converted to ketones 5a and 5b by Dess-Martin oxidation.

Method D is a modification of Yavari's vinyltriphenylphosphonium salt mediated synthesis of 4-carboxymethylcoumarins (Yavari, I.; Hekmat-Shoar, R.; Zonouzi, A. Tetrahedron Letters 1998, 39, 2391-2392). Ketones 5b and 5c were obtained by heating the equimolar amounts of 4-substituted methyl 4-oxo-bytynoates, 8-hydroxyjulolidine 15 and PPh₃ in acetonitrile. Chemical yields were substrate dependent: 59% for 5c (R=Ph), 13% for 5b (R=Cy).

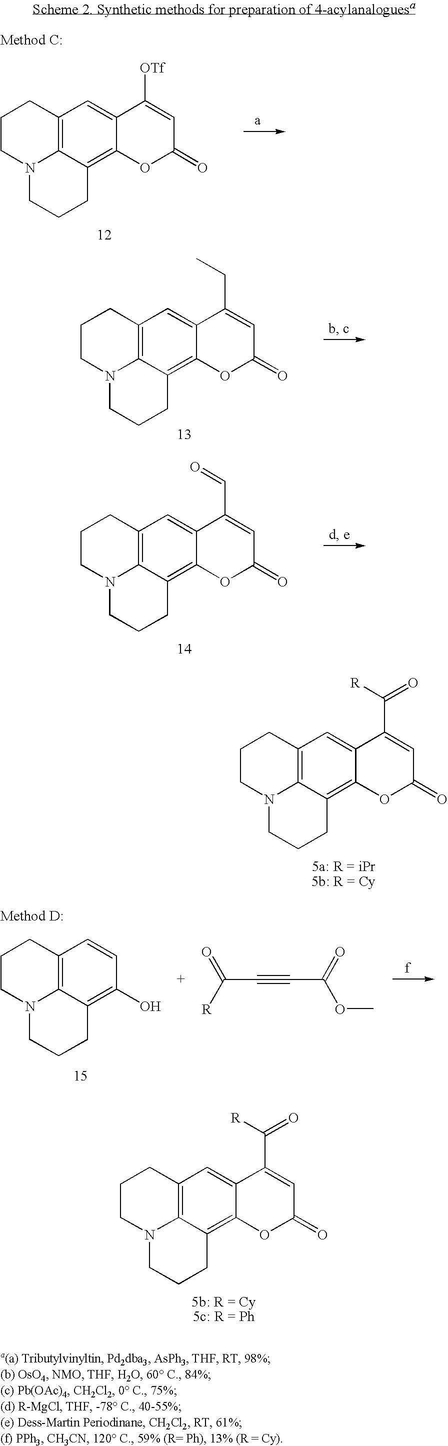

^{a}(a) Tributylvinyltin, Pd₂dba₃, AsPh₃, THF, RT, 98%; (b) OsO₄, NMO, THF, H₂O, 60° C., 84%; (c) Pb(OAc)₄, CH₂Cl₂, 0° C., 74%; (d) R—MgCl, THF, −78° C., 40-55%; (e) Dess-Martin Periodinane, CH₂Cl₂, RT, 61%; (f) PPh₃, CH₃CN, 120° C., 59% (R=Ph), 13% (R=Cy).

3-Substituted analogues 5e was prepared by bromination of probe 5 (Br₂, ACOH, CH₂Cl₂), while 5f required an additional step, namely Suzuki coupling of the 3-bromonalogue 5e with phenylboronic acid (PdCl₂dppf, Na₂CO₃, DMF, H₂O).

Cyclopentenone analogue 5d was prepared as shown in Scheme 3. The von Pechmann condensation of 3,5-dicarbomethoxycyclopentane-1,2-dione (Buu-Hoi, N. P.; Lavit-Lamy, D. Bull. Soc. Chim. Fr. 1962, 773-775) with 8-hydroxyjulolidine 15 was achieved by heating the equimolar mixture of the reactants at 110° C. without solvent (35% yield). Addition of various amounts of InCl₃ did not increase the yield of the condensation. Dealkoxycarbonylation of the β-ketoester 16 using LiCl in wet DMSO afforded 5d in 75% yield. All synthesized ketones were converted to the corresponding alcohols by Luche reduction (NaBH₄/CeCl₃) in MeOH/CH₂Cl₂.

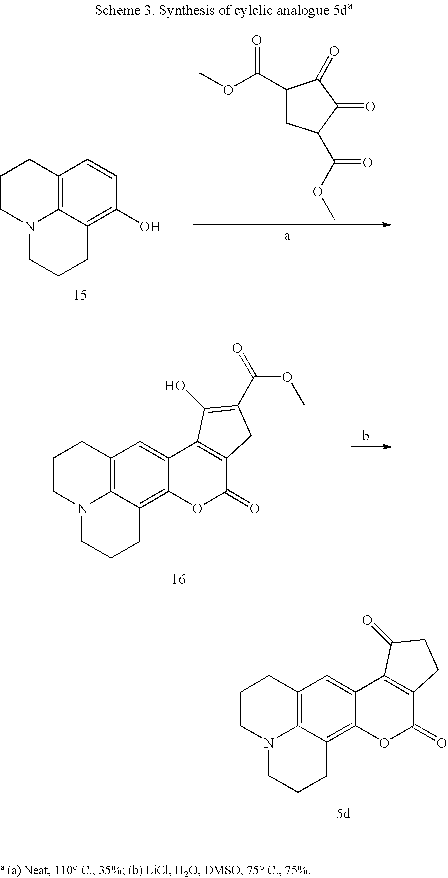

Detailed experimental protocols can be found in the Materials and Methods.

Materials and Methods

¹H and ¹³C NMR spectra were recorded on Bruker 300 or 400 Fourier transform NMR spectrometers. Spectra were recorded in CDCl₃ solutions referenced to TMS or the solvent residual peak unless otherwise indicated. IR spectra were taken as neat for licuids on NaCl plates using a Perkin-Elmer 1600 FTIR spectrometer. Low Resolution and High Resolution Mass Spectra were obtained on a JOEL JMS-HX110 HF mass spectrometer. Flash chromatography was performed on SILICYCLE silica gel (230-400 mesh). All chemicals were purchased from Aldrich and used as received. All reactions were monitored by Thin Layer Chromatography.

Ultraviolet spectra were measured on a Perkin Elmer UV/VIS/NIR spectrophotometer Lambda 19 and recorded in pH 7 doubly deionized water (2% DMSO or 4% acetonitrile). Recorded λₘₐₓ is that of the longest wavelength transition. Fluorescence measurements were taken on a Jobin Yvon Fluorolog fluorescence spectrofluorometer in pH 7 doubly deionized water (2% DMSO or 4% acetonitrile).

Synthesis of Probes 5a-5i and the Corresponding Alcohols 17a-17i

Synthesis of Methylketone Probes: Method A

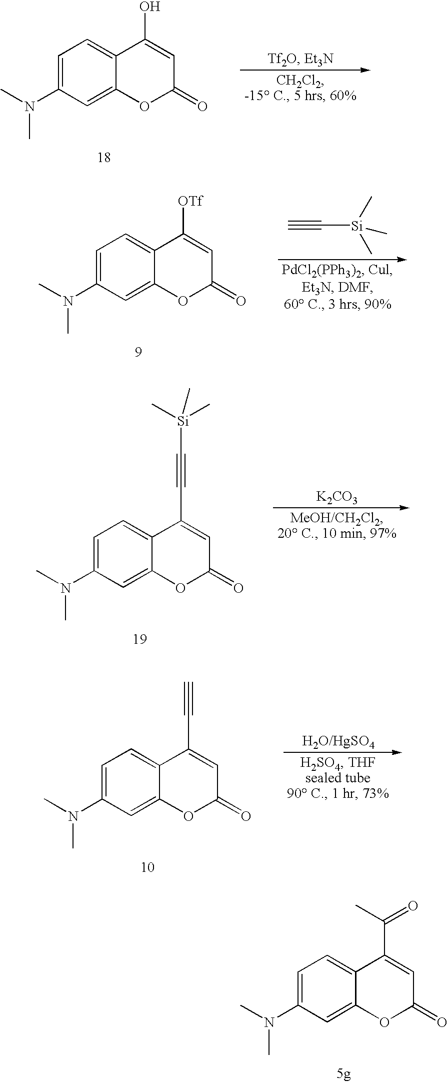

### 7-Dimethylamino-4-trifluoromethanesulfonyloxy-coumarin (9).

4-Hydroxycoumarin 18 (700 mg, 3.41 mmol), prepared according to literature (Knierzinger, A.; Wolfbeis, O. S. J. Heterocyclic Chem. 1980, 17, 2217-261), and triethylamine (688 μl, 4.95 mmol) were dissolved in dry CH₂Cl₂ (35 ml) under argon. The mixture was cooled to −20° C. and trifluoromethanesulfonic anhydride (746 μl, 4.43 mmol) was added dropwise. After 5 hrs at −10° C., the mixture was diluted with hexanes-EtOAc 2:1. The resulting solution was passed through a silica column and the product was washed from the column using hexanes-EtOAc 2:1. The solvent was removed in vacuo to afford the triflate 9 (692 mg, 60%).

NMR ¹H (300 MHz, CDCl₃) δ ppm:

7.44 (d, 1H, J=9.0 Hz); 6.66 (dd, 1H, J1=9.0 Hz, J2=2.4 Hz); 6.52 (d, 1H, J=2.4 Hz); 6.09 (s, 1H); 3.10 (s, 6H).

NMR ¹³C (75 MHz, CDCl₃) δ ppm:

161.1; 158.1; 155.9; 154.1; 123.2;. 118.4 (q, J_{CF}=318.8 Hz); 109.6; 102.4; 98.9; 98.7; 40.1.

IR (NaCl, cm⁻¹) 3086; 2924; 1722; 1616; 1528; 1425; 1397; 1224; 1138; 883; 594.

HRMS (FAB): 337.0233 (C₁₂H₁₀O₅NF₃S, M; calc. 337.0232).

### 7-Dimethylamino-4-trimethylsilanylethynyl-coumarin (19)

PdCl₂(PPh₃)₂ (33 mg, 0.05 mmol), CuI (9 mg, 0.05 mmol), Et₃N (330 μl, 2.37 mmol) and (trimethylsilyl)acetylene (328 μl, 2.37 mmol) were added to a solution of triflate 9 (400 mg, 1.19 mmol) in dry DMF (12 ml) under argon. The resulting solution was heated to 60° C. and allowed to react 2 hours. The mixture was then cooled, diluted with water, and extracted with CH₂Cl₂. The organic fractions were then combined and dried over MgSO₄. Removal of solvent in vacuo and purification of the residue by column chromatography on silica gel (CH₂Cl₂) afforded product 19 (307 mg, 90%).

NMR ¹H (300 MHz, CDCl₃) δ ppm:

7.59 (d, 1H, J=8.9 Hz); 6.63 (dd, 1H, J1=8.9 Hz, J2=2.4 Hz); 6.46 (d, 1H, J=2.4 Hz); 6.19 (s, 1H); 3.06 (s, 6H); 0.32 (s, 9H).

NMR ¹³C (75 MHz, CDCl₃) δ ppm:

161.4; 155.7; 153.1; 137.0; 127.3; 112.4; 109.0; 108.2; 107.4; 98.3; 97.8; 40.1; −0.4.

IR (NaCl, cm⁻¹) 2963; 2902; 1707; 1620; 1582; 1525; 1392; 1276; 1246; 1160; 857; 844; 815.

HRMS (FAB): 286.1254 (C₁₆H₂₀O₂NSi, M+H; calc. 286.1263).

### 7-Dimethylamino-4-ethynyl-coumarin (10)

Powdered K₂CO₃ (320 mg) was added to a solution of 19 (316 mg, 1.11 mmol) in MeOH—CH₂Cl₂ 5:1 (36 ml). The mixture was stirred at room temperature until the reaction was complete (10 min). Reaction mixture was diluted with CHCl₃, filtered, and washed with brine. The resultant organic layers were combined and dried over MgSO₄, after which the solvent was removed in vacuo. Purification by column chromatography on silica gel (eluent gradient: CH₂Cl₂ to CH₂Cl₂-EtOAc 98:2) afforded terminal alkyne 10 (229 mg, 97%).

NMR ¹H (300 MHz, CDCl₃) δ ppm:

7.61 (d, 1H, J=8.9 Hz); 6.62 (dd, 1H, J1=8.9 Hz, J2=2.5 Hz); 6.46 (d, 1H, J=2.5 Hz); 6.25 (s, 1H); 3.64 (s, 1H); 3.07 (s, 6H).

NMR ¹³C (75 MHz, CDCl₃) δ ppm:

161.2; 155.7; 153.2; 136.3; 127.2; 113.3; 109.1; 108.1; 97.9; 88.0; 77.7; 40.1.

IR (NaCl, cm¹) 3230; 2905; 2101; 1697; 1616; 1583; 1524; 1394; 1247; 1152; 840; 812.

HRMS (FAB): 214.0867 (C₁₃H₁₂O₂N, M+H; calc. 214.0868).

### 4-Acetyl-7-dimethylamino-coumarin (5g)

HgSO₄ (300 mg, 1.01 mmol) was added to a solution of 10 (216 mg, 1.01 mmol) in THF-acetone 5:1 (25 ml), followed by addition of 0.4 M H₂SO₄ (5.05 ml, 2.02 mmol). The reaction mixture was heated in a sealed tube at 90° C. for 1 hr. After cooling to room temperature, a spatula tip of NaHCO₃ was added and the mixture was evaporated to dryness. MgSO₄ was added and the residual solids were washed thoroughly with CHCl₃. The solvent was evaporated and the residue purified by column chromatography on silica gel (CH₂Cl₂-Et₂O 95:5). Recrystallization from hexanes-CHCl₃ yielded ketone 5g (171 mg, 73%).

NMR ¹H (300 MHz, CDCl₃) δ ppm:

7.71 (d, 1H, J=9.1 Hz); 6.60 (dd, 1H, J1=9.1 Hz, J2=2.6 Hz); 6.51 (d, 1H, J=2.6 Hz); 6.28 (s, 1H); 3.06 (s, 6H); 2.58 (s, 3H).

NMR ¹³C (75 MHz, CDCl₃) δ ppm:

199.8; 161.8; 156.8; 153.0; 149.8; 127.3; 109.5; 109.4; 104.5; 98.2; 40.0; 29.4.

IR (NaCl, cm⁻¹) 3073; 2912; 1725; 1687; 1629; 1579; 1522; 1407; 1373; 1272; 1239; 1133; 1018; 868; 811.

HRMS (FAB): 231.0905 (C₁₃H₁₃O₃N, M; calc. 231.0895).

Synthesis of Methylketone Probes: Method B

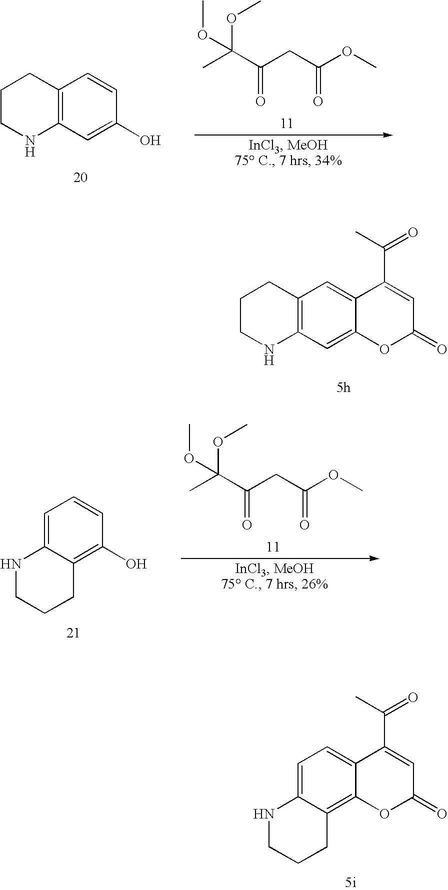

### 4-Acetyl-5,6,7,8-tetrahydro-1-oxa-8-aza-anthracen-2-one (5h)

Phenol 20 was obtained by BBr₃ mediated demethylation of 7-methoxy-1,2,3,4-tetrahydroquinoline, prepared from 6-methoxy-indanone by a literature procedure (Torisawa, Y.; Nishi, T.; Minamikawa, J. Bioorg. Med. Chem. Lett. 2002, 12, 387-390).

Solution of phenol 20 (200 mg, 1.34 mmol), methyl 4,4-dimethoxy-3-oxovalerate 11 (268 mg, 1.41 mmol) and InCl₃ (311 mg, 1.41 mmol) in MeOH (2.7 ml) was stirred in a sealed tube for 7 hrs at 75° C. The cooled mixture was then diluted with CHCl₃, washed with brine and dried over MgSO₄. The solvent was evaporated and the residue was purified by column chromatography on silica gel (eluent gradient: CH₂Cl₂ to CH₂Cl₂-EtOAc 97:3). The isolated product was recrystallized from CHCl₃-haxanes to yield ketone 5h (111 mg, 34%).

NMR ¹H (300 MHz, CDCl₃) δ ppm:

7.37 (s, 1H); 6.33 (s, 1H); 6.21 (s, 1H); 4.60 (bs, 1H); 3.38 (m, 2H); 2.76 (t, 2H, J=6.2 Hz); 2.56 (s, 3H); 1.93 (m, 2H).

NMR ¹³C (75 MHz, CDCl₃) δ ppm:

200.1; 161.8; 155.4; 150.3; 148.8; 126.5; 118.9; 108.5; 104.9; 99.3; 41.6; 29.6; 26.8; 21.2.

IR (NaCl, cm⁻¹) 3353; 2859; 1719; 1681; 1624; 1553; 1521; 1487; 1348; 1322; 1298; 1233; 1145; 836.

LRMS (FAB): 244 (C₁₄H₁₄O₃N, M+H).

### 8-Acetyl-1,2,3,4-tetrahydro-5-oxa-1-aza-phenantren-6-one (5i)

Phenol 21 (265 mg, 1.78 mmol), prepared by hydrogenation of 5-hydroxyquinoline (Atkins R. L.; Bliss, D. E. J. Org. Chem. 1987, 43, 1975-1980), was condensed with 11 by the procedure used for the preparation of 5h to yield 5i (112 mg, 26%).

NMR ¹ H (300 MHz, CDCl₃) δ ppm:

7.41 (d, 1H, J=8.8 Hz); 6.36 (d, 1H, J=8.8 Hz); 6.21 (s, 1H); 4.53 (bs, 1H); 3.37 (m, 2H); 2.88 (t, 2H, J=6.4 Hz); 2.56 (s, 3H); 1.96 (m, 2H).

NMR ¹³C (75 MHz, CDCl₃) δ ppm:

200.1; 161.9; 153.9; 150.9; 148.7; 124.8; 111.3; 108.1; 107.4; 105.1; 41.1; 29.7; 20.5; 19.9.

IR (NaCl, cm⁻) 3353; 2950; 2848; 1708; 1694; 1615; 1587; 1559; 1531; 1398; 1349; 1229; 1208; 1182; 1120; 815.

LRMS (FAB): 244 (C₁₄H₁₄O₃N, M+H).

Synthesis of 4-Acylanalogues: Method C

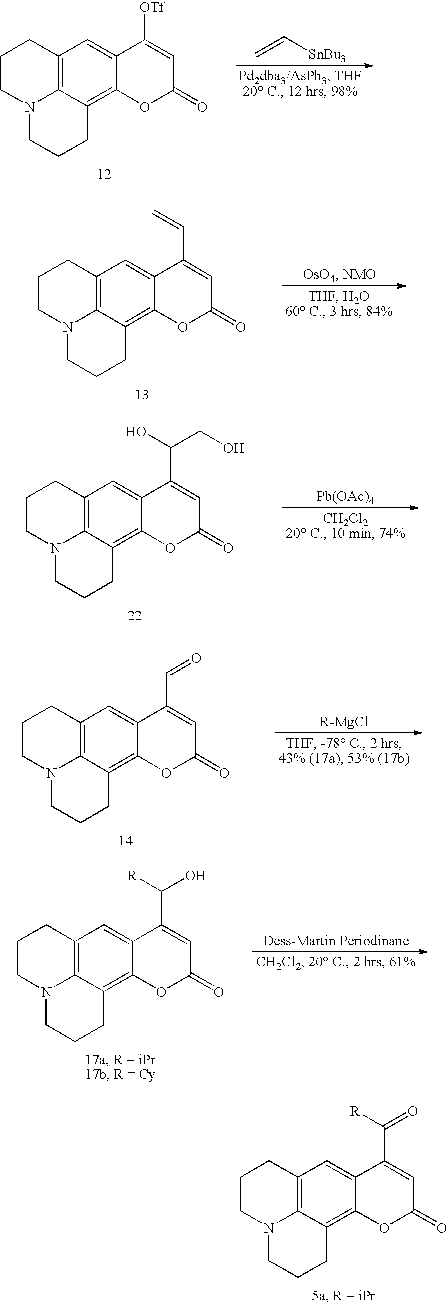

### 8-Vinyl-2,3,5,6-tetrahydro-1H,4H-11-oxa-3a-aza-benzo[de]anthracen-10-one (13)

Pd₂dba₃ (31 mg, 0.04 mmol) and AsPh₃ (83 mg, 0.27 mmol) were dissolved in dry THF (13 ml) under argon. After 10 min at RT, triflate 12 (Coleman, R. S.; Madaras, M. L. J. Org. Chem. 1998, 63, 5700-5703) (528 mg, 1.36 mmol) and tributylvinyltin (429 μl, 1.42 mmol) were added. The resultant solution was stirred for 12 hrs at RT. Aqueous KF was added and after 20 minutes the mixture was extracted with EtOAc. The organic layer was dried over MgSO₄ and concentrated. The crude product was then purified by column chromatography on silica gel (CH₂Cl₂-EtOAc 98:2) to provide pure 13 (355 mg, 98%).

NMR ¹H (300 MHz, CDCl₃) δ ppm:

7.04 (s, 1H); 6.91 (dd, 1H, J1=17.3 Hz, J2=10.9 Hz); 6.08 (s, 1H); 5.89 (dd, 1H, J1=17.3 Hz, J2=1.1 Hz); 5.58 (dd, 1H, J1=10.9 Hz, J2=1.1 Hz); 3.25 (m, 4H); 2.89 (t, 2H, J=6.5 Hz); 2.77 (t, 2H, J=6.3 Hz); 1.97 (m, 4H).

NMR ¹³C (75 MHz, CDCl₃) δ ppm:

163.3; 151.8; 151.7; 146.3; 131.5; 122.3; 122.0; 118.4; 107.6; 107.4; 104.3; 50.3; 49.9; 28.1; 21.9; 21.0; 20.9.

IR (NaCl, cm⁻¹) 2947; 2839; 1701; 1614; 1555; 1516; 1434; 1354; 1311; 1182; 834.

HRMS (FAB): 268.1348 (C₁₇H₁₈O₂N, M+H; calc. 268.1338).

### 8-(1,2-Dihydroxy-ethyl)-2,3,5,6-tetrahydro-1H,4H-11-oxa-3a-aza-benzo[de]anthracen-10-one (22)

To a solution of 13 (300 mg, 1.12 mmol) in THF-H₂O 2:1 (45 ml), 4-methylmorpholine N-oxide (211 mg, 1.80 mmol) and 2.5 wt % OsO₄ in t-BuOH (703 μl, 0.06 mmol) were added. The solution was then warmed to 60° C. and stirred at this temperature for 3 hrs. NaHSO₃ (0.5 g) was added to the cooled mixture followed by the addition of saturated aqueous NaHCO₃. The resulting mixture was extracted with CHCl₃. The combined organic fractions were dried over MgSO₄. Following evaporation of solvent, the residue was purified by column chromatography on silica gel (eluent gradient: CH₂Cl₂-EtOAc-MeOH 8:2:0 to 50:48:2) to afford desired diol 22 (284 mg, 84%).

NMR ¹H (300 MHz, CDCl₃) δ ppm:

6.96 (s, 1H); 6.25 (d, 1H, J=0.7 Hz); 5.11 (m, 1H); 3.93 (m, 1H); 3.66 (m, 1H); 3.25 (m, 4H); 2.86 (t, 2H, J=6.5 Hz); 2.75 (t, 2H, J=6.3 Hz); 2.69 (d, 1H, J=3.7 Hz); 2.21 (dd, 1H, J1=7.9 Hz, J2=4.5 Hz); 1.97 (m, 4H).

NMR ¹³C (75 MHz, CD₃OD) δ ppm:

165.3; 159.1; 152.5; 147.4; 122.4; 120.2; 107.7; 107.6; 105.1; 71.6; 67.6; 50.9; 50.4; 28.7; 22.6; 21.7; 21.5.

IR (NaCl, cm⁻¹) 3379; 2938; 2838; 1685; 1610; 1553; 1522; 1437; 1374; 1311; 1205; 1179; 1125.

HRMS (FAB): 301.1320 (C₁₇H₁₉O₄N, M; calc. 301.1314).

### 10-oxo-2,3,5,6-tetrahydro-1H,4H-11-oxa-3a-aza-benzo[de]anthracene-8-carbaldehyde (14)

Pb(OAc)₄ (23 mg, 0.05 mmol) was added to a solution of 22 (15 mg, 0.05 mmol) in dry CH₂Cl₂ (4 ml) under argon at 0° C. After 10 minutes at 0° C., the solution was diluted with CHCl₃, washed with H₂O and 10% aqueous K₂CO₃, and dried over MgSO₄. The solvent was removed in vacuo and the residue was purified by column chromatography on silica gel (CH₂Cl₂-EtOAc 98:2) to afford desired aldehyde 14 (10 mg, 74%).

NMR ¹H (300 MHz, CDCl₃) δ ppm:

10.00 (s, 1H); 7.89 (s, 1H); 6.37 (s, 1H); 3.31 (m, 4H); 2.88 (t, 2H, J=6.5 Hz); 2.78 (t, 2H, J=6.2 Hz); 1.99 (m, 4H).

NMR ¹³C (75 MHz, CDCl₃) δ ppm:

192.8; 162.1; 152.3; 146.4; 143.9; 122.8; 119.2; 115.8; 106.7; 103.7; 50.0; 49.5; 27.7; 21.4; 20.4; 20.4.

IR (NaCl, cm⁻¹) 2938; 2841; 2739; 1711; 1704; 1608; 1582; 1550; 1520; 1430; 1373; 1308; 1163; 1119.

HRMS (FAB): 269.1043 (C₁₆H₁₅O₃N, M; calc. 269.1052).

### 8-(1-Hydroxy-2-methyl-propyl)-2,3,5,6-tetrahydro-1H,4H-11-oxa-3a-aza-benzo[de]anthracen-10-one (17a)

A 2 M solution of isopropylmagnesium chloride in Et₂O (54 μl, 0.11 mmol) was added to a solution of 14 (20 mg, 0.07 mmol) in dry THF (1 ml) at −78° C. under argon. After 2 hrs at −78° C., the reaction was quenched with saturated aqueous NH₄Cl and extracted with EtOAc. The organic layer was dried over Na₂SO₄, evaporated, and the residue was purified by column chromatography on silica gel (eluent gradient: CH₂Cl₂-EtOAc 100:0 to 95:5). Recrystallization from CHCl₃-hexanes provided pure alcohol 17a (10 mg, 43%).

NMR ¹H (300 MHz, CDCl₃) δ ppm:

7.00 (s, 1H); 6.13 (s, 1H); 4.70 (m, 1H); 3.25 (m, 4H); 2.86 (t, 2H, J=6.5 Hz); 2.76 (t, 2H, J=6.3 Hz); 2.09 (m, 1H), 1.97 (m, 4H); 1.04 (d, 3H, J=6.9 Hz); 0.94 (d, 3H, J=6.7 Hz).

NMR ¹³C (75 MHz, CDCl₃) δ ppm:

162.9; 157.9; 151.4; 145.6; 121.4; 117.9; 107.0; 106.4; 105.5; 74.8; 49.9; 49.4; 33.3; 27.8; 21.5; 20.6; 20.4; 20.1; 16.4.

IR (NaCl, cm⁻¹) 3420; 2931; 2842; 1696; 1610; 1554; 1521; 1437; 1311; 1205; 1181; 1136; 1018; 730.

LRMS (FAB): 314 (C₁₉H₂₄O₃N, M+H).

### 8-(Cyclohexyl-hydroxy-methyl)-2,3,5,6-tetrahydro-1H,4H-11-oxa-3a-aza-benzo[de]anthracen-10-one (17b)

Addition of a 2 M solution of cyclohexylmagnesium chloride in Et₂O (54 μl, 0.11 mmol) to a solution of 14 (20 mg, 0.07 mmol) in THF (1 ml) using the procedure described for 17a afforded 17b (14 mg, 53%).

NMR ¹H (300 MHz, CDCl₃) δ ppm:

7.02 (s, 1H); 6.10 (s, 1H); 4.68 (m, 1H); 3.25 (m, 4H); 2.86 (t, 2H, J=6.5 Hz); 2.77 (t, 2H, J=6.3 Hz); 1.97 (m, 5H); 1.70 (m, 6H); 1.19 (m, 5H).

NMR ¹³C (75 MHz, CDCl₃) δ ppm:

162.9; 157.6; 151.4; 145.6; 121.5; 117.9; 107.0; 106.6; 105.7; 74.6; 49.9; 49.4; 43.1; 30.3; 27.8; 27.1; 26.3; 26.2; 25.9; 21.5; 20.6; 20.5.

IR (NaCl, cm⁻¹) 3421; 2929; 2850; 1690; 1610; 1552; 1521; 1437; 1370; 1311; 1177; 909; 731.

LRMS (FAB): 354 (C₂₂H₂₈O₃N, M+H).

### 8-Isobutyryl-2,3,5,6-tetrahydro-1H,4H-11-oxa-3a-aza-benzo[de]anthracen-10-one (5a)

A solution of Dess-Martin periodinane in CH₂Cl₂ (257 μl, 15 wt %, 0.12 mmol) was added dropwise to a solution of 17a (30 mg, 0.01 mmol) in dry CH₂Cl₂ (2 ml) at RT under argon. After 2 hrs at RT, the resulting mixture was passed through a silica column and the product was washed from the column using CH₂Cl₂. The solvent was removed and the product was recrystallized from CHCl₃-hexanes to afford 5a (18 mg, 61%).

NMR ¹H (300 MHz, CDCl₃) δ ppm:

6.85 (s, 1H); 5.99 (s, 1H); 3.27 (m, 4H); 3.13 (sep, 1H, J=6.9 Hz); 2.87 (t, 2H, J=6.5 Hz); 2.72 (t, 2H, J=6.2 Hz); 1.96 (m, 4H); 1.20 (d, 6H, J=6.9 Hz).

NMR ¹³C (75 MHz, CDCl₃) δ ppm:

206.7; 162.0; 152.6; 151.9; 146.4; 123.0; 118.8; 106.9; 105.1; 104.6; 49.9; 49.5; 39.9; 27.6; 21.3; 20.4; 20.3; 17.7.

IR (NaCl, cm⁻¹) 2934; 2841; 1719; 1701; 1613; 1585; 1546; 1522; 1432; 1372; 1311; 1167; 1006.

LRMS (FAB): 312 (C₁₉H₂₂O₃N, M+H).

Synthesis of 4-Acylanalogues: Method D

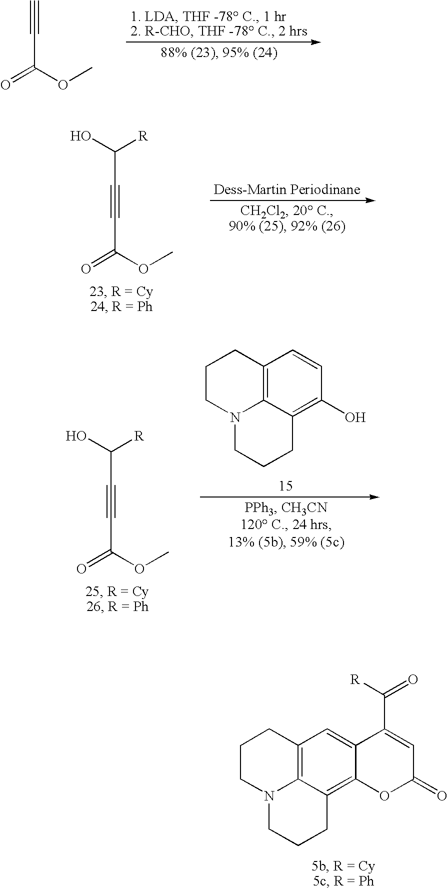

### Methyl 4-cyclohexyl-4-hydroxy-but-2-ynoate (23)

Butyllithium in hexanes (5.68 ml, 1.6 M sol., 9.09 mmol) was added to a solution of diisopropylamine (1.21 ml, 8.66 mmol) in dry THF (35 ml) at 0° C. under argon. After 10 min at 0° C., the LDA solution was cooled to −78° C. Methyl propiolate (0.74 ml, 8.24 mmol) was then added dropwise. After stirring the mixture for 1 hr at −78° C., cyclohexane-carboxaldehyde (1.06 ml, 8.65 mmol) was added. The reaction temperature was maintained at −78° C. for 2 hrs. The reaction was quenched by an addition of H₂O. The resulting mixture was diluted with EtOAc, washed with saturated aqueous NH₄Cl, and concentrated in vacuo. Purification by column chromatography on silica gel (CH₂Cl₂) afforded the pure product 23 (1.43 g, 88%).

NMR ¹H (300 MHz, CDCl₃) δ ppm:

4.27 (t, 1H, J=6.1 Hz); 3.78 (s, 3H); 2.07 (d, 1H, J=6.1 Hz); 1.76 (m, 6H); 1.20 (m, 5H).

NMR ¹³C (75 MHz, CDCl₃) δ ppm:

153.8; 87.5; 66.9; 52.8; 43.6; 28.3; 28.0; 26.1; 25.7.

IR (NaCl, cm⁻¹) 3416; 2929; 2854; 2235; 1718; 1451; 1435; 1251; 1016; 752.

LRMS (FAB): 197 (C₁₁H₁₇O₃, M+H).

### Methyl 4-hydroxy-4-phenyl-but-2-ynoate (24)

Compound 24 was prepared from benzaldehyde (0.88 ml, 8.66 mmol) and methyl propiolate (0.74 ml, 8.24 mmol) as described for the preparation of 23. Column chromatography on silica gel (eluent gradient: hexanes-EtOAc 95:5 to 8:2) provided 24 (1.49 g, 95%). Spectral data are consistent with those previously published (Arcadi, A.; Bernocchi, E.; Burini, A.; Cacchi S.; Marinelli F.; Pietroni B. Tetrahedron 1988, 44, 481-490).

### Methyl 4-cyclohexyl-4-oxo-but-2-ynoate (25)

A solution of Dess-Martin periodinane in CH₂Cl₂ (6.70 ml, 15 wt %, 3.21 mmol) was added dropwise to a solution of 23 (484 mg, 2.47 mmol) in dry CH₂Cl₂ (10 ml) at RT under argon. After 1 hr, Na₂S₂O₃ (2 g) and saturated aqueous NaHCO₃ (20 ml) were added. The resulting mixture was stirred for 15 min, extracted with CH₂Cl₂, and dried over MgSO₄. Following evaporation of solvent, the residue was purified by column chromatography on silica gel (CH₂Cl₂) to afford desired product 25 (433 mg, 90%). Spectral data are consistent with literature (Naka, T.; Koide, K. Tetrahedron Lett. 2003, 44, 443-4417).

### Methyl 4-oxo-4-phenyl-but-2-ynoate (26)

Dess-Martin oxidation of 24 (743 mg, 3.91 mmol) proceeded as described for 25 to yield 26 (677 mg, 92%). Spectral data are consistent with literature (Aitken, R. A.; Herion, H.; Janosi, A.; Karodia, N.; Raut, S. V.; Seth, S.; Shannon, I. J.; Smith, F. C. J. Chem. Soc. Perkin Trans. 1 1994, 17, 2467-2472).

### 8-Cyclohexanecarbonyl-2,3,5,6-tetrahydro-1H,4H-11-oxa-3a-aza-benzo[de]anthracen-10-one (5b)

Solution of 25 (224 mg, 1.15 mmol) in CH₃CN (5 ml) was added dropwise to a solution of 8-hydroxyjulolidine 15 (214 mg, 1.10 mmol) and PPh₃ (288 mg, 1.10 mmol) in CH₃CN (10 ml) at −5° C. After 10 min at −5° C., the resulting mixture was warmed in a sealed tube to 120° C. and maintained at this temperature for 24 hrs. The reaction mixture was cooled down and solvent removed in vacuo. The residue was subjected to multiple rounds of column chromatography (eluent gradient: CH₂Cl₂-EtOAc 100:0 to 95:5 and hexanes-EtOAc 9:1) and recrystallized from CHCl₃-hexanes to afford 5b (50 mg, 13%).

NMR ¹H (300 MHz, CDCl₃) δ ppm:

6.84 (s, 1H); 5.98 (s, 1H); 3.26 (m, 4H); 2.91 (m, 3H); 2.72 (t, 2H, J=6.2 Hz); 1.96 (m, 6H); 1.75 (m, 3H); 1.32 (m, 5H).

NMR ¹³C (75 MHz, CDCl₃) δ ppm:

206.2; 162.1; 152.8; 151.9; 146.3; 123.0; 118.7; 106.9; 104.9; 104.7; 49.9; 49.5; 49.5; 28.0; 27.6; 25.7; 25.4; 21.3; 20.4; 20.3.

IR (NaCl, cm⁻¹) 2931; 2851; 1718; 1613; 1585; 1546; 1521; 1432; 1371; 1312; 1164; 1142; 730.

LRMS (FAB): 352 (C₂₂H₂₆O₃N, M+H).

### 8-Benzoyl-2,3,5,6-tetrahydro-1H,4H-11-oxa-3a-aza-benzo[de]anthracen-10-one (5c)

Reaction of 26 (206 mg, 1.09 mmol) with 15 (203 mg, 1.04 mmol) and PPh₃ (273 mg, 1.04 mmol) in CH₃CN (15 ml) under conditions similar to those used for the preparation of 5b provided 5c (212 mg, 59%).

NMR ¹H (300 MHz, CDCl₃) δ ppm:

7.95 (m, 2H); 7.63 (m, 1H); 7.48 (m, 2H); 6.73 (s, 1H); 5.93 (s, 1H); 3.28 (m, 4H); 2.92 (t, 2H, J=6.5 Hz); 2.63 (t, 2H, J=6.2 Hz); 1.95 (m, 4H).

NMR ¹³C (75 MHz, CDCl₃) δ ppm:

194.3; 161.6; 152.2; 151.9; 146.5; 135.3; 134.6; 130.1; 128.9; 123.1; 118.7; 106.9; 106.1; 105.4; 49.9; 49.5; 27.5; 21.2; 20.4; 20.3.

IR (NaCl, cm⁻¹) 2936; 2844; 1716; 1670; 1614; 1586; 1547; 1522; 1433; 1371; 1311; 1260; 1166; 728.

LRMS (FAB): 346 (C₂₂H₂₀O₃N, M+H).

Synthesis of 3-Substituted Analogues

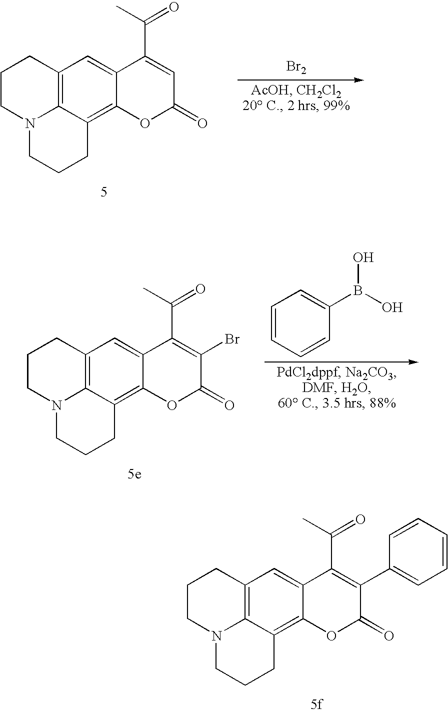

### 8-Acetyl-9-bromo-2,3,5,6-tetrahydro-1H,4H-11-oxa-3a-aza-benzo[de]anthracen-10-one (5e)

Br₂ (19 μl, 0.37 mmol) in AcOH (5 ml) was added dropwise to a solution of 5 (100 mg, 0.35 mmol) in AcOH—CH₂Cl₂ 1:1 (5 ml) over 2 hrs at RT. After 15 minutes the mixture was diluted with H₂O (20 ml), neutralized with aqueous 10% NaOH, and extracted with CHCl₃. The combined organic layers were dried over Na₂SO₄ and concentrated. The residue was purified by column chromatography on silica gel (CH₂Cl₂-EtOAc 99:1) and recrystallized from CHCl₃-hexanes to yield 5e (146 mg, 99%).

NMR ¹H (300 MHz, CDCl₃) δ ppm:

6.55 (s, 1H); 3.28 (m, 4H); 2.87 (t, 2H, J=6.4 Hz); 2.71 (t, 2H, J=6.2 Hz); 2.59 (s, 3H); 1.96 (m, 4H).

NMR ¹³C (75 MHz, CDCl₃) δ ppm:

200.2; 158.0; 154.1; 151.0; 146.6; 121.8; 119.3; 106.9; 104.7; 96.0; 50.0; 49.5; 30.4; 27.6; 21.1; 20.2; 20.2.

IR (NaCl, cm⁻¹) 2941; 2840; 1715; 1617; 1521; 1437; 1350; 1311; 1204; 1166; 1144.

LRMS (FAB): 362 (C₁₇H₁₇O₃BrN, M+H).

### 8-Acetyl-9-phenyl-2,3,5,6-tetrahydro-1H,4H-11-oxa-3a-aza-benzo[de]anthracen-10-one (5f)

Bromide 5e (42 mg, 0.12 mmol) was mixed with phenylboronic acid (22 mg, 0.17 mmol), PdCl₂dppf (3 mg, 0.003 mmol), Na₂CO₃ (61 mg, 0.58 mmol), H₂O (285 μl) and DMF (1.2 ml) under argon. The resulting mixture was heated to 60° C. and stirred until completion (3.5 hrs) The cooled mixture was then diluted with water and extracted with CH₂Cl₂. The combined organic fractions were dried over MgSO4. Following evaporation of solvent, the residue was purified by column chromatography on silica gel (CH₂Cl₂) and recrystallized from CHCl₃-hexanes to afford desired product 5f (73 mg, 88%).

NMR ¹H (300 MHz, CDCl₃) δ ppm:

7.36 (m, 5H); 6.69 (s, 1H); 3.29 (m, 4H); 2.92 (t, 2H, J=6.4 Hz); 2.72 (t, 2H, J=6.1 Hz); 1.98 (m, 4H); 1.95 (s, 3H).

NMR ¹³C (75 MHz, CDCl₃) δ ppm:

203.2; 161.8; 151.3; 151.3; 146.1; 133.7; 130.2; 128.5; 128.5; 122.5; 118.8; 115.5; 106.9; 104.4; 49.9; 49.5; 31.1; 27.6; 21.3; 20.5; 20.4.

IR (NaCl, cm⁻¹) 2943; 2845; 1707; 1616; 1549; 1521; 1444; 1311; 1163; 912; 732.

LRMS (FAB): 360 (C₂₃H₂₂O₃N, M+H).

Synthesis of Cyclic Analogue 5d

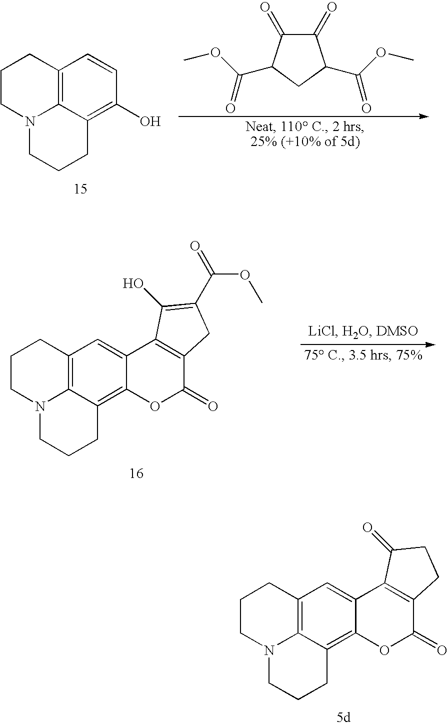

### Methyl 1-hydroxy-4-oxo-3,4,7,8,10,11-hexahydro-6H,9H-5-oxa-8a-aza-benzo[fg]cyclopenta[a]anthracene-2-carboxylate (16)

A mixture of finely powdered 8-hydroxyjulolidine 15 (123 mg, 0.63 mmol) and dicarbomethoxycyclopentane-1,2-dione (142 mg, 0.66 mmol), prepared according to literature (Hauser, C. R.; Hudson, B. E. Org. React. 1942, 1, 284), was heated in a vial at 110° C. under argon for 2 hrs. The cooled mixture was dissolved in CH₂Cl₂ and subjected to a column chromatography on silica gel to afford 5d (19 mg, 10%) and 16 (56 mg, 25%).

NMR ¹H (300 MHz, CDCl₃) δ ppm:

10.42 (bs, 1H); 7.65 (s, 1H); 3.87 (s, 3H); 3.52 (s, 2H); 3.26 (m, 4H); 2.90 (t, 2H, J=6.5 Hz); 2.79 (t, 2H, J=6.3 Hz); 1.98 (m, 4H).

NMR ¹³C (75 MHz, CDCl₃) δ ppm:

169.0; 168.5; 160.0; 152.3; 146.6; 145.7; 121.8; 120.0; 118.5; 108.8; 106.9; 104.5; 51.6; 50.0; 49.5; 31.8; 27.7; 21.5; 20.7; 20.6.

IR (NaCl, cm⁻¹) 2946; 2844; 1715; 1656; 1612; 1551; 1517; 1445; 1377; 1310; 1219; 1119; 1068; 907; 732.

LRMS (FAB): 354 (C₂₀H₂₀O₅N, M+H).

### 2,3,7,8,10,11-Hexahydro-6H,9H-5-oxa-8a-aza-benzo[fg]cyclopenta[a]anthracene-1,4-dione (5d)

A solution of 16 (56 mg, 0.16 mmol), LiCl (14 mg, 0.32 mmol) and H₂O (6 μl, 0.32 mmol) in DMSO (2 ml) was stirred at 75° C. for 3.5 hrs. The resulting mixture was cooled, diluted with EtOAc-hexanes 1:1 (100 ml), washed with H₂O, and dried over Na₂SO₄. The solvent was evaporated, the residue was purified by column chromatography on silica gel (CH₂C1₂), and then recrystallized from CHCl₃-hexanes to provide 5d (35 mg, 75%).

NMR ¹H (300 MHz, CDCl₃) δ ppm:

7.85 (s, 1H); 3.26 (m, 4H); 2.91 (m, 4H); 2.78 (t, 2H, J=6.3 Hz); 2.71 (m, 2H); 1.97 (m, 4H).

NMR ¹³C (75 MHz, CDCl₃) δ ppm:

208.1; 162.0; 151.8; 145.8; 143.4; 138.2; 121.2; 119.0; 106.7; 103.3; 50.0; 49.5; 36.6; 27.6; 23.0; 21.4; 20.6; 20.5.

IR (NaCl, cm⁻³) 2936; 2837; 1707; 1612; 1560; 1516; 1445; 1380; 1306; 1256; 1181; 1125; 732.

LRMS (FAB): 296 (Cl₈H₁₈O₃N, M+H).

Synthesis of Alcohols 17c-17f

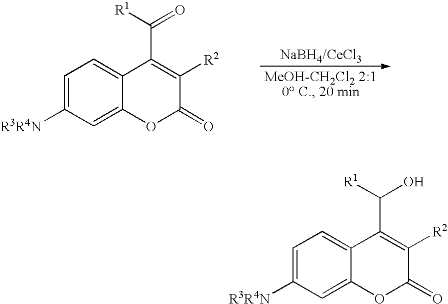

General Procedure

CeCl₃.7H₂O (48 mg, 0.13 mmol) was added to a solution of ketone (0.10 mmol) in MeOH—CH₂Cl₂ 2:1 (6 ml) at 0° C., followed by addition of NaBH₄ (20 mg, 0.52 mmol). After 20 minutes, the reaction was quenched with a saturated aqueous solution of NH₄Cl and extracted with CHCl₃. The organic layer was dried over MgSO₄, evaporated, and the crude product was purified by column chromatography on silica gel (eluent gradient: CH₂Cl₂-EtOAc 98:2 to 8:2). Recrystallization from CHCl₃-hexanes provided pure alcohol.

### 8-(Hydroxy-phenyl-methyl)-2,3,5,6-tetrahydro-1H,4H-11-oxa-3a-aza-benzo[de]anthracen-10-one (17c)

Yield: 87%

NMR ¹H (300 MHz, CDCl₃) δ ppm:

7.35 (m, 5H); 6.86 (s, 1H); 6.37 (s, 1H); 5.96 (d, 1H, J=3.6 Hz); 3.21 (m, 4H); 2.85 (t, 2H, J=6.5 Hz); 2.63 (m, 2H); 2.30 (d, 1H, J=3.6 Hz); 1.91 (m, 4H).

NMR ¹³C (75 MHz, CDCl₃) δ ppm:

163.1; 156.6; 151.3; 145.5; 140.7; 128.9; 128.4; 127.1; 121.9; 117.9; 106.7; 106.1; 105.7; 72.3; 49.8; 49.4; 27.6; 21.4; 20.5; 20.4.

IR (NaCl, cm⁻²) 3385; 2938; 2843; 1685; 1611; 1554; 1521; 1437; 1374; 1311; 1205; 1175; 1119; 732; 700.

LRMS (FAB3): 348 (C₂₂H₂₂O₃N, M+H).

### 1-Hydroxy-2,3,7,8,10,11-hexahydro-1H,6H,9H-5-oxa-8a-aza-benzo[fg]cyclopenta[a]anthracene-4-one (17d)

Yield: 82%

NMR ¹H (300 MHz, CDCl₃) δ ppm:

7.23 (s, 1H); 5.40 (m, 1H); 3.24 (m, 4H); 2.93 (m, 1H); 2.84 (t, 2H, J=6.6 Hz); 2.77 (t, 2H, J=6.4 Hz); 2.68 (m, 1H); 2.55 (m, 1H); 2.01 (m, 6H).

NMR ¹³C (75 MHz, CDCl₃) δ ppm:

161.7; 155.1; 152.1; 145.3; 121.7; 120.5; 118.3; 107.1; 106.4; 76.5; 50.0; 49.5; 34.1; 27.6; 27.4; 21.5; 20.6; 20.6.

IR (NaCl, cm⁻¹) 3408; 2939; 2851; 1686; 1607; 1559; 1517; 1441; 1378; 1311; 1184; 1121; 1069; 749.

LRMS (FAB): 298 (C₁₈H₂₀O₃N, M+H).

### 9-Bromo-8-(1-hydroxy-ethyl)-2,3,5,6-tetrahydro-1H,4H-11-oxa-3a-aza-benzo[de]anthracen-10-one (17e)

Yield: 55%, reduction required 1.5 hrs at RT

NMR ¹H (300 MHz, CDCl₃) δ ppm:

7.88 (s, 1H); 5.53 (m, 1H); 3.24 (m, 4H); 2.78 (m, 5H); 1.96 (m, 4H); 1.60 (d, 3H, J=6.8 Hz).

NMR ¹³C (75 MHz, CDCl₃) δ ppm:

158.5; 155.6; 150.2; 145.7; 123.7; 118.3; 106.6; 106.4; 102.3; 71.5; 50.0; 49.5; 27.9; 21.5; 21.5; 20.5; 20.4.

IR (NaCl, cm⁻¹) 3441; 2941; 2842; 1693; 1611; 1516; 1429; 1353; 1310; 1167; 1148.

LRMS (FAB): 364 (C₁₇H₉O₃BrN, M+H).

### 8-(1-Hydroxy-ethyl)-9-phenyl-2,3,5,6-tetrahydro-1H,4H-11-oxa-3a-aza-benzo [de]anthracen-10-one (17f)

Yield: 80%, reduction required 2 hrs at RT

NMR ¹H (300 MHz, CDCl₃) δ ppm:

7.78 (s, 1H); 7.38 (m, 3H); 7.21 (m, 2H); 4.91 (m, 1H); 3.26 (m, 4H); 2.91 (t, 2H, J=6.5 Hz); 2.79 (t, 2H, J=6.3 Hz); 1.99 (m, 4H); 1.90 (d, 1H, J=3.7 Hz); 1.58 (d, 3H, J=6.7 Hz).

NMR ¹³C (75 MHz, CDCl₃) δ ppm:

162.4; 153.1; 151.1; 145.2; 135.1; 129.9; 128.5; 127.7; 124.6; 119.0; 117.7; 106.8; 105.9; 68.2; 49.9; 49.4; 27.9; 23.1; 21.7; 20.7; 20.5.
IR (NaCl, cm⁻³) 3395; 2936; 2841; 1677; 1612; 1550; 1518; 1442; 1369; 1310; 1192; 1137; 732; 700.

LRMS (FAB): 362 (C₂₃H₂₄O₃N, M+H).

### 7-Dimethylamino-4-(1-hydroxy-ethyl)-coumarin (17g)

Yield: 91%

NMR ¹H (300 MHz, CDCl₃) δ ppm:

7.43 (d, 1H, J=9.0 Hz); 6.59 (dd, 1H, J1=9.0 Hz, J2=2.6 Hz); 6.50 (d, 1H, J=2.6 Hz); 6.28 (s, 1H); 5.14 (q, 1H, J=6.5 Hz); 3.04 (s, 6H); 2.12 (bs, 1H); 1.56 (d, 3H, J=6.5 Hz).

NMR ¹³C (75 MHz, CDCl₃) δ ppm:

162.8; 159.7; 156.0; 152.5; 124.7; 108.9; 106.7; 105.0; 98.4; 65.9; 40.1; 23.5.

IR (NaCl, cm⁻¹) 3406; 2979; 2926; 1691; 1616; 1528; 1407; 1372; 1328; 1119; 1000; 854.

HRMS (FAB): 234.1138 (C₁₃H₁₆O₃N, M+H; calc. 234.1130).

### 4-(1-Hydroxy-ethyl)-5,6,7,8-tetrahydro-1-oxa-8-aza-anthracen-2-one (17h)

Yield: 86%

NMR ¹H (300 MHz, CDCl₃) δ ppm:

7.13 (s, 1H); 6.32 (s, 1H); 6.25 (s, 1H); 5.11 (m, 1H); 4.50 (bs, 1H); 3.37 (t, 2H, J=5.5 Hz); 2.78 (t, 2H, J=6.2 Hz); 2.02 (d, 1H, 3.7 Hz); 1.95 (m, 2H); 1.56 (d, 3H, J=6.8 Hz).

NMR ¹³C (75 MHz, CDCl₃) δ ppm:

162.6; 159.1; 154.6; 148.0; 124.2; 118.1; 107.2; 104.8; 99.5; 66.0; 41.6; 26.9; 23.6; 21.4.

IR (NaCl, cm⁻¹) 3345; 2927; 2838; 1678; 1619; 1563; 1527; 1490; 1321; 1301; 1177; 1121; 835.

LRMS (FAB): 246 (C₁₄H₁₆O₃N, M+H).

### 8-(1-Hydroxy-ethyl)-1,2,3,4-tetrahydro-5-oxa-1-aza-phenantren-6-one (17i)

Yield: 95%

NMR ¹H (300 MHz, CDCl₃) δ ppm:

7.23 (d, 1H, J=8.7 Hz); 6.38 (d, 1H, J=8.7 Hz); 6.30 (d, 1H, J=0.7 Hz); 5.15 (m, 1H); 4.40 (bs, 1H); 3.37 (m, 2H); 2.87 (t, 2H, J=6.5 Hz); 2.10 (d, 1H, J=3.9 Hz); 1.96 (m, 2H); 1.55 (d, 3H, J=6.6 Hz).

NMR ¹³C (75 MHz, CDCl₃) δ ppm:

163.1; 160.5; 153.0; 148.0; 122.2; 110.8; 107.4; 107.2; 104.2; 65.7; 41.1; 23.6; 20.6; 19.9.

IR (NaCl, cm⁻¹) 3359; 2932; 2848; 1686; 1615; 1592; 1563; 1398; 1332; 1291; 1119; 1021; 731.

LRMS (FAB): 246 (C₁₄H₁₆O₃N, M+H).

Photophysical Characterization

Extinction coefficients reported are the average of triplicate measurements of the lowest energy wavelength transition at three different concentrations. Fluorescence quantum yields are the average of three independent quantum yield determinations and are determined by excitation at 340, 365, or 420 nm using either 9, 10-diphenylanthracene in EtOH (Heinrich, G.; Schoof, S.; Gusten, H. J. Photochem. 1974/75, 3, 312-320) or coumarin 6 in EtOH (Reynolds, G. A.; Drexhage, K. H. Opt. Commun. 1975, 13, 222.) as fluorescence standards. The described photophysical data is represented on Tables 4 and 5.

TABLE 4
Photophysical properties for the first
generation of fluorogenic substrates^{‡}
KETONE

KETONE

| | |
|---|---|
| | 1 |
| 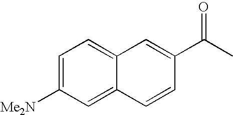 | |
| | 2 |
| 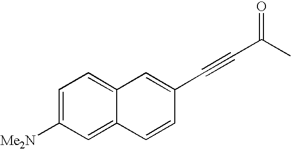 | |
| | 3 |
| 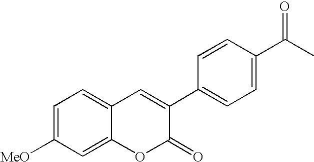 | |
| | 4 |
| 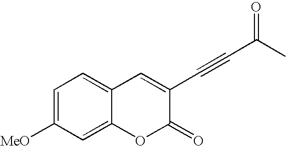 | |
| | 5 |
| 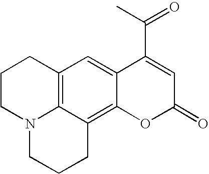 | |
| | 6 |
| 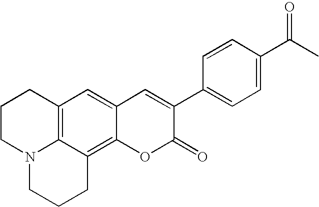 | |
| | 7 |
| 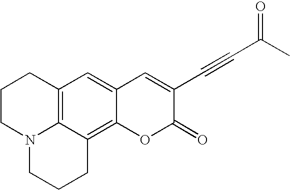 | |

| COMPOUND | λₘₐₓ | ε (M⁻¹cm⁻¹) | λₑₘ | Φ |
|---|---|---|---|---|
| 1 | 362 | 15,500 ± 900 | 524 | 0.139 ± 0.007^{b} |
| 2 | 372 | 8,400 ± 800 | 470 | 0.0023 ± 0.0001^{b} |
| 3 | 317 | 9,600 ± 700 | 462 | 0.041 ± 0.001^{a} |
| 4 | 368 | 25,000 ± 1000 | 416 | 0.005 ± 0.001^{b} |
| 5 | 436 | 12,900 ± 200 | 520 | 0.00080 ± 0.00008^{c} |
| 6 | 435 | 18,400 ± 900 | 511 | 0.0003 ± 0.0001^{c} |
| 7 | 464 | 33,000 ± 3000 | 512 | 0.009 ± 0.001^{c} |

ALCOHOL

| | |
|---|---|
| | 27 |
| 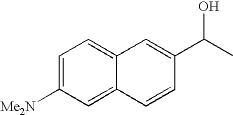 | |
| | 28 |
| 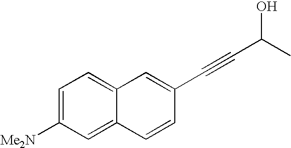 | |
| | 29 |
| 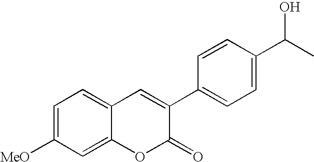 | |
| | 30 |
| 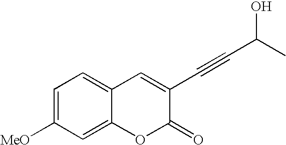 | |
| | 31 |
| 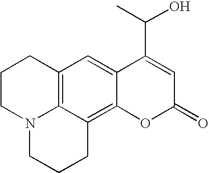 | |
| | 32 |
| 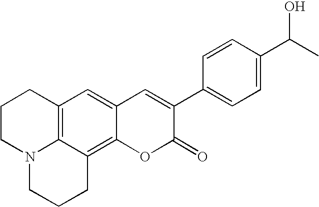 | |
| | 33 |
| 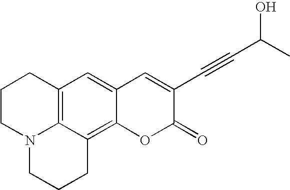 | |

| | | | | |
|---|---|---|---|---|
| 27 | 337 | 1,550 ± 80 | 432 | 0.59 ± 0.02^{b} |
| 28 | 313 | 17,000 ± 2000 | 440 | 0.41 ± 0.05^{a} |
| 29 | 342 | 15,300 ± 3000 | 429 | 0.54 ± 0.09^{b} |
| 30 | 346 | 20,300 ± 600 | 420 | 0.79 ± 0.08^{b} |
| 31 | 398 | 14,500 ± 400 | 509 | 0.29 ± 0.04^{c} |
| 32 | 422 | 13,400 ± 500 | 509 | 0.36 ± 0.07^{c} |
| 33 | 429 | 24,000 ± 500 | 508 | 0.50 ± 0.08^{c} |

^{‡}all measurements performed in pH 7 doubly deionized water (4% acetonitrile).
^{a}relative to 9,10-diphenyl anthracene as a standard (excited at 340 nm);
^{b}relative to 9,10-diphenyl anthracene as a standard (excited at 365 nm);
^{c}relative to coumarin 6 as a standard (excited at 420 nm)

TABLE 5
Photophysical properties for the second
generation of fluorogenic substrates^{‡}

KETONE

| | |
|---|---|
| | 5a |
| 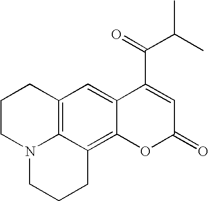 | |
| | 5b |
| 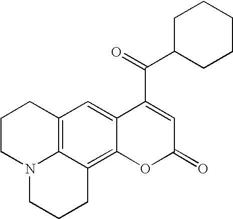 | |
| | 5c |
| 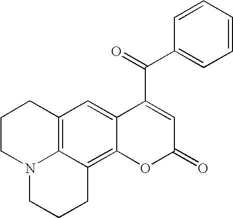 | |
| | 5d |
| 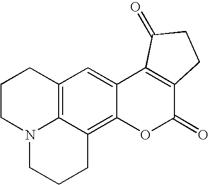 | |
| | 5e |
| 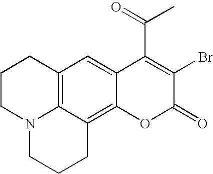 | |
| | 5f |
| 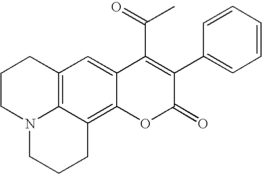 | |
| | 5g |
| 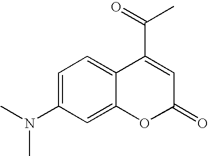 | |
| | 5h |
| 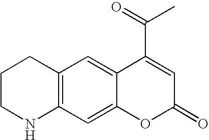 | |
| | 5i |
| 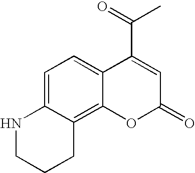 | |

| | λₘₐₓ | | λₑₘ | |
|---|---|---|---|---|
| COMPOUND | (nm) | ε (M⁻¹cm⁻¹) | (nm) | Φ |
| 5a | 431 | 8,500 ± 500 | 514 | 0.0012 ± 0.0002^{c} |
| 5b | 440 | 5,200 ± 700 | 598 | 0.0015 ± 0.0001^{c} |
| 5c | 427 | 11,200 ± 300 | 608 | 0.0014 ± 0.0002^{c} |
| 5d | 464 | 3,700 ± 100 | — | 0.0038 ± 0.0002^{c} |
| 5e | 442 | 19,000 ± 1,000 | — | 0.0010 ± 0.0003^{c} |
| 5f | 437 | 17,000 ± 700 | 595 | 0.0010 ± 0.0002^{c} |
| 5g | 436 | 11,500 ± 200 | 501 | 0.0054 ± 0.0008^{c} |
| 5h | 409 | 11,500 ± 300 | 487 | 0.0133 ± 0.0001^{c} |
| 5i | 404 | 9,300 ± 100 | 497 | 0.0054 ± 0.0004^{c} |

ALCOHOL

| | |
|---|---|
| | 17a |
| 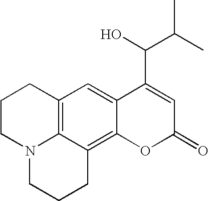 | |
| | 17b |
| 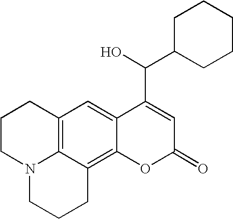 | |
| | 17c |
| 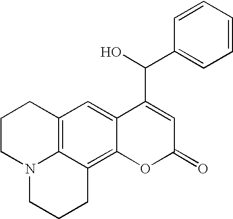 | |
| | 17d |
| 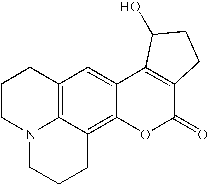 | |
| | 17e |
| 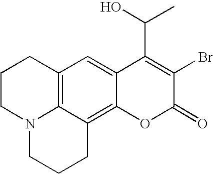 | |

| | |
|---|---|
| | 17f |
| 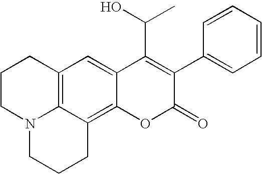 | |

| | |
|---|---|
| | 17g |
| 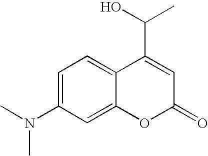 | |
| | 17h |
| 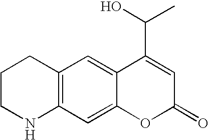 | |

| | |
|---|---|
| | 17i |
| 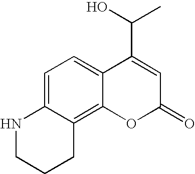 | |

| | | | | |
|---|---|---|---|---|
| 17a | 406 | 11,300 ± 600 | 503 | 0.59 ± 0.03^{c} |
| 17b | 415 | 7,200 ± 300 | 502 | 0.26 ± 0.05^{c} |
| 17c | 409 | 12,500 ± 100 | 507 | 0.32 ± 0.06^{c} |
| 17d | 400 | 12,300 ± 100 | 508 | 0.43 ± 0.08^{c} |
| 17e | 427 | 16,700 ± 600 | 524 | 0.05 ± 0.01^{c} |
| 17f | 409 | 15,000 ± 400 | 524 | 0.12 ± 0.02^{c} |
| 17g | 402 | 16,100 ± 300 | 489 | 0.15 ± 0.03^{c} |
| 17h | 376 | 14,900 ± 400 | 482 | 0.72 ± 0.10^{b} |
| 17i | 371 | 13,300 ± 200 | 490 | 0.49 ± 0.10^{b} |

^{‡}all measurements performed in pH 7 doubly deionized water (4% acetonitrile).
^{b}relative to 9,10-diphenylanthracene as a standard (excited at 365 nm);
^{c}relative to coumarin 6 as a standard (excited at 420 nm)

Enzymology with Purified Enzymes:

AKR1C2 and AKR1C3 Selective Probes: Probes (5a-5i) were examined as substrates for the four purified human HSD isozymes (AKR1C1-AKR1C4) under standard assay conditions [catalytic quantities of enzyme, an excess of cofactor (NADPH)]. The initial reaction rates were used to derive standard kinetic parameters (k_{cat} and Kₘ, Materials and Methods).

It was found that all four human isozymes catalyzed the reduction of parent probe 5 by NADPH, albeit at significantly different rates. Probe 5 showed preference for AKR1C2 and AKR1C3 over AKR1C1 and AKR1C4 by two orders of magnitude in terms of catalytic efficiency (k_{cat}/Kₘ). These results are significant in view of the importance of AKR1C2 and AKR1C3 in steroid hormone action (Table 3).

TABLE 3
Kinetic parameters for fluorogenic substrates.

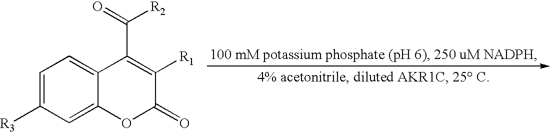

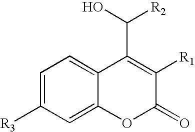

| | | | Kₘ | K_{cat} | |
|---|---|---|---|---|---|
| | STRUCTURE | isozyme | (μM) | (min⁻¹) | CE^{a} |
| 1 | 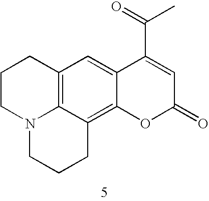 | 1C1 1C2 1C3 1C4 | 24.4 ± 2.3 7.3 ± 0.4 1.1 ± 0.1 20.6 ± 1.9 | 1.1 14.8 7.5 0.38 | 0.046 2.1 6.9 0.020 |
| 2 | 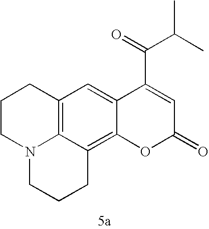 | 1C1 1C2 1C3 1C4 | no activity | | |
| 3 | 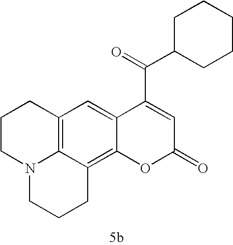 | 1C1 1C2 1C3 1C4 | no activity | | |
| 2 | 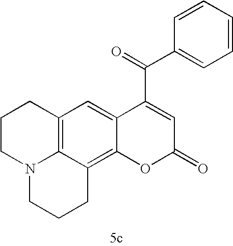 | 1C1 1C2 1C3 1C4 | 5.5 ± 0.7 3.0 ± 0.2 0.051 ± 0.005 6.2 ± 0.7 | 1.5 23.2 5.4 0.62 | 0.27 7.8 106 0.10 |
| 3 | 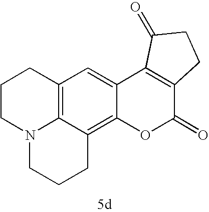 | 1C1 1C2 1C3 1C4 | 4.0 ± 0.6 4.5 ± 0.6 0.13 ± | 1.6 0.40 7.2 no activity | 0.39 0.08 56 |
| 4 | 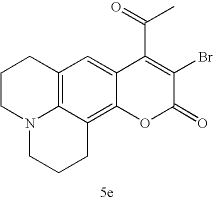 | 1C1 1C2 1C3 1C4 | no activity | | |
| 5 | 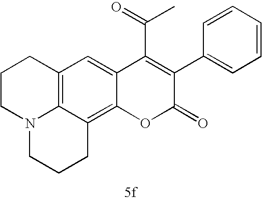 | 1C1 1C2 1C3 1C4 | no activity | | |
| 6 | 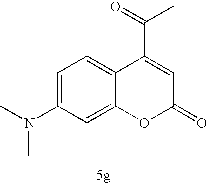 | 1C1 1C2 1C3 1C4 | 21.0 ± 1.3 7.2 ± 0.6 4.3 ± 0.3 14.8 ± 0.9 | 2.0 22.2 8.7 2.1 | 0.10 3.0 2.1 0.14 |
| 5 | 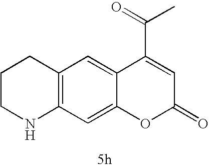 | 1C1 1C2 1C3 1C4 | 43.3 ± 4.4 4.3 ± 0.2 | no activity NA 0.59 9.3 no activity | 0.015 2.2 |
| 6 | 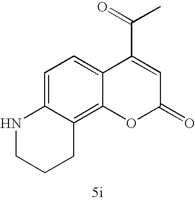 | 1C1 1C2 1C3 1C4 | 35.3 ± 2.9 4.8 ± 0.3 8.3 ± 0.9 40.2 ± 3.4 | 6.6 29.2 6.9 1.4 | 0.19 6.0 0.84 0.036 |

^{a}C.E.—Catalytic efficiency (k_{cat}/Kₘ) measured in units of min⁻¹ μM⁻¹;
no activity = 10 μg enzyme produced less than 0.1 nmol product per min (determined fluorimetrically).

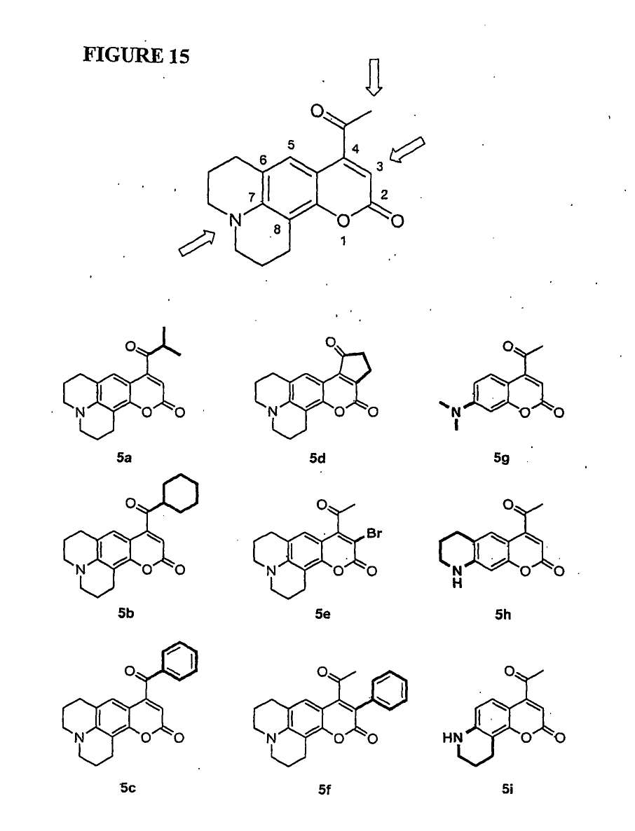

As expected, structural changes at the three selected positions (FIG. 15) resulted in dramatic changes in both activity and selectivity. The ketone group was found to be a “sensitive area” where introduction of bulky alkyl groups (such as iso-propyl or cyclohexyl) completely abolished the activity. On the other hand, phenyl ketone 5c proved to be an excellent probe, showing high selectivity for AKR1C3. The Kₘ value for this isozyme was in the nanomolar range (51 nM), two orders of magnitude lower than for other isozymes. Excellent selectivity of phenyl ketone probe 5c was also seen in terms of catalytic efficiency (Table 3).

Cyclic probe 5d represents an interesting compound wherein the conformational orientation of the ketone group was fixed by the formation of a five-membered ring. Notably, this probe also showed high selectivity for AKR1C3.

Introduction of a substituent at position C-3 of the coumarine core led to a complete loss of activity as demonstrated by 3-bromo and 3-phenyl derivatives 5e and 5f, respectively. These compounds were not accepted as substrates by any of the tested 3a-HSD enzymes.

Examination of the substitution patterns at and near the nitrogen atom yielded interesting results. The suspicion that the two nitrogen-containing rings play an important role in the enzyme-substrate recognition was confirmed. In contrast to probe 5, dimethylamino analog 5g (lacking the two six-membered rings) was not particularly selective, showing only a small preference for AKR1C2 and AKR1C3. On the other hand, “truncated analogs” 5h and 5i, containing only one saturated ring, proved highly selective. In fact, they exhibited complementary profiles: probe 5h demonstrated excellent selectivity for AKR1C3, while compound 5i preferred AKR1C2 (Table 3).

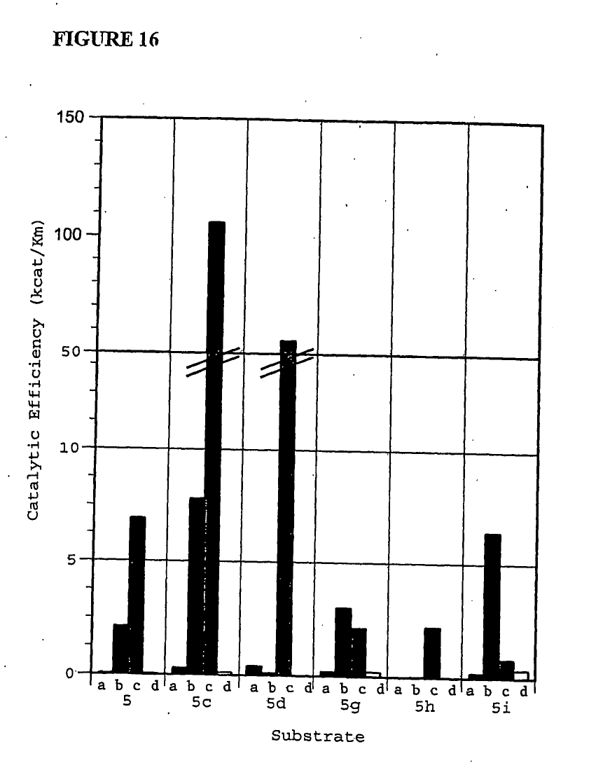

When it is considered that human AKR1Cl-AKR1C4 share in excess of 84% sequence identity, the prospect of finding isozyme selective probes at the onset of our studies seemed unlikely. Nevertheless, as summarized in a graphical form in FIG. 16, three probes were identified with high selectivity for AKR1C3 (5c, 5d, 5h) and one probe with good selectivity for AKR1C2 (5i).

In terms of both activity and selectivity, phenyl ketone 5c is an excellent substrate. Remarkably, this probe is a far superior substrate for 1C3 isozyme (Kₘ=0.05 μM, k_{cat}=5.93 min⁻¹) when compared to likely physiological substrates such as 5α-dihydrotestosterone (Kₘ=26 μM, k_{cat}=0.25 min⁻¹).

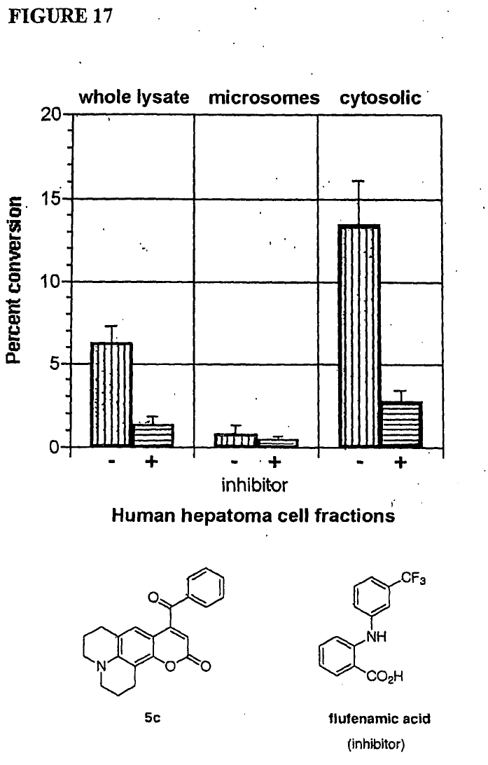

Selectivity of Phenyl Ketone Probe 5c in Cellular Lysates: The selectivity of phenyl ketone probe 5c in human hepatoma cells (HepG2), which are known to express all four AKR1C isozymes in the cytoplasm, was tested. Liver is the hub of metabolic activity in higher organisms and thus these cells possess a broad repertoire of oxidoreductases. An issue may be non-selective reduction of probes with microsomes, which are organelles enriched with redox enzymes. Following one hour incubation of probe 5c with both cytosolic and microsomal fractions prepared from HepG2 cells, the resulting mixtures were analyzed fluorimetrically. It was found that probe 5c was stable in the presence of microsomes while enzymatic reduction occurred in the cytoplasmic fraction (FIG. 17). Moreover, reduction by the cytoplasmic extract was suppressed by flufenamic acid, a known inhibitor of the AKR1C isozymes (Penning, T. M.; Talalay, P. Proc Natl Acad Sci USA 1983, 4504-4508).

These results support the thesis that fluorogenic probes that have no structural relationship to steroid can be developed which are highly selective for AKR1C isozymes.

Conclusion

This investigation resulted in the discovery of probes selective for AKR1C isozymes. Probes 5c, 5d, and 5h showed excellent selectivity for AKR1C3 (type 5 17β-HSD) while probe 5i had good preference for AKR1C2 (type 3 3α-HSD). It was found that phenyl ketone probe 5c was selective for AKR1C3 in lysates of hepatoma cells (HepG2). Thus, the activity of these enzymes could be measured optically in cellular extracts, known to contain several hundred oxidoreductase enzymes.

These probes provide the opportunity for imaging AKR1C activity in living cells and tissues. This possibility is of significant importance considering the physiological role of these enzymes, as well as their elevated expression in some tumors.

Enzymatic Activity Determinations with Purified Dehydrogenases

Initial Screening with Fluorogenic Substrates: Screening of the first fluorogenic substrates for enzymatic activity has been described above. In short, 200 μL enzymatic assay volumes containing 100 mM potassium phosphate buffer (pH 7), 250 μM NAD(P)H cofactor, and 30-50 μM of ketones (1-7) were incubated for 12 hours on a black FALCON 96-well plate. Formation of the alcohol reduction product was determined by reading the fluorescence arising from excitation at the corresponding alcohol at either 340 nm (27-30) or 440 nm (31-33).

Substrate tolerance of AKR1Cs: Isozyme Activity with 5a-5i: Activity of the second generation of fluorogenic substrates with the AKR1C isozymes was determined as follows. To a STARNA semi-micro fluorimeter cell (with 4 polished windows) was added 100 μL of 1 M potassium phosphate buffer (pH 6), 840 μL doubly deionized water, and 20 μL of 12.5 mM NADPH. After mixing the aqueous components thoroughly, 20 μL of acetonitrile was added as a cosolvent and mixed well. 20 μL of 2.5 mM second generation fluorogenic ketone in acetonitrile (5a-5i) was then added and mixed. 4 μL of undiluted AKR1C (provided generously by the Penning lab at concentrations of 2.5 mg/mL) were then added to the cuvette for a total of 10 μg purified enzyme in the 1 mL assay volume. Fluorescence arising from the respective alcohol reduction product was then monitored over the course of 5-10 minutes.

Determination of Steady State Kinetic Parameters for the AKR1Cs

Binding constant and catalytic rates (Kₘ and k_{cat}) of the second generation of fluorogenic substrates was determined as follows. To a STARNA semi-micro fluorometer cell (with 4 polished windows) was added 100 μL of 1 M potassium phosphate buffer (pH 6), 840 μL doubly deionized water, and 20 AL of 12.5 mM NADPH. After mixing the aqueous components thoroughly, 20 μL of acetonitrile was added as a cosolvent and mixed well. 20 μL of the second generation fluorogenic ketone (5a-5i) was then added and mixed to achieve assay concentrations of 5Kₘ to Kₘ/5. To initiate the reduction, 2 or 4 μL of diluted AKR1C (1:2 to 1:100, depending on the kinetics of a particular isozyme's reduction of a substrate) was then added to the cuvette. Fluorescence arising from the respective alcohol reduction product was then monitored over the course of 3 minutes (Excitation and emission band pass slits both at 4 nm, lamp 700 V, λ_{exc} 410 nm, λₑₘ 510). The rate of product formation, expressed in units of nanomoles per minute, were calculated according to previously published procedures (Wierzchowski, J.; Dafeldecker, W. P.; Holmquist, B.; Vallee, B. L. Anal. Biochem. 1989, 178, 57-62):

initial rate=[nₛₜ×(Fₜ−F₀)/(Fₛₜ)]/t (1)

where Fₜ and F₀ represent the fluorescence at times t and 0 minutes, nₛₜ is the nanomoles of product in a known concentration of product, and Fₛₜ is the fluorescence resulting from nₛₜ of product. Kinetic parameters were approximated by GraFit (Erithacus Software, Surrey, UK) nonlinear regression analysis program to fit the untransformed data to a hyperbolic function as originally described (Wierzchowski, J.; Dafeldecker, W. P.; Holmquist, B.; Vallee, B. L. Anal. Biochem. 1989, 178, 57-62). Reported enzymatic kinetic parameters are the average of three independent determinations from three different preparations of substrate and enzyme.

Substrate for Monitoring Reductase Activity (Via Reduction of Ketones or Aldehydes to Alcohols)

Initially a product calibration curve is made by plotting fluorescence against varying concentrations of aldehyde/ketone and alcohol under normal assay conditions. Aside from allowing for quantification of kinetic parameters, the calibration will is instructive as to the sensitivity of product detectable when accounting for background fluorescence of the measurement instrument.

When monitoring product formation, in most cases the increase in fluorescence (“off/on” switch) may be followed arising from the alcohol by exciting at the probes respective absorption maxima and monitoring at their respective emission maxima. In the cases that the alcohol is the less fluorescent of the two compounds (“on/off” switch), it is more favorable to detect enzymatic reduction by a decrease in fluorescence (e.g. see MK62/VB440, MONAL62/VB439, VB463/VB464, VB431/VB432 below).

Detecting reduction of substrate VB468 can be done by exciting at 280 nm and monitoring increase in fluorescence at 354 nm. Alternatively, one may monitor the decrease in fluorescence from the substrate VB3468 by exciting at 342 nm and monitoring the decrease in fluorescence at 473 nm. This alternative is available for VB468/VB467, DY111/DY511, Coumarin 334/VB93, MONAL62/VB439, VB463/VB464, MF-2-91/VB427 and VB431/VB432.

All molecules shown in Table 6 are soluble in DMSO, methanol, and acetonitrile at concentrations of 2.5 mM unless indicated by an asterisk (*), in which cases they may be dissolved in concentrations of 1 mM. The fluorescence spectra are pH independent in the range of 5-9. The molecules are stable in common biological buffers used (Tris-HCl, sodium and potassium phosphate buffers).

TABLE 6
Probes suitable for monitoring reductase
activity.

| | |
|---|---|
| | MK62 |
| 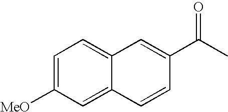 | |
| | VB468 |
| 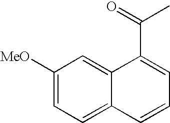 | |
| | VB476 |
| 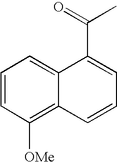 | |
| | DY111 |
| 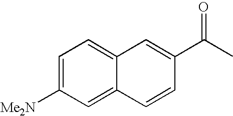 | |
| | VB412 |
| 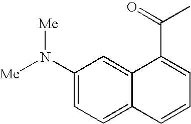 | |
| | VB460 |
| 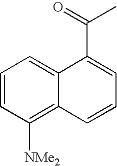 | |
| | DYX1 |
| 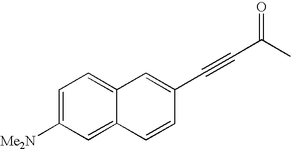 | |
| | VB11 * |
| 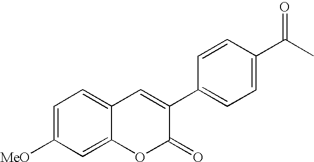 | |

| | |
|---|---|
| | VB14 |
| 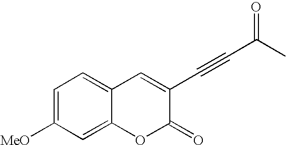 | |

| | |
|---|---|
| | VB35 * |
| 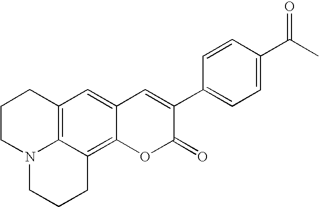 | |

| | |
|---|---|
| | VB40 |
| 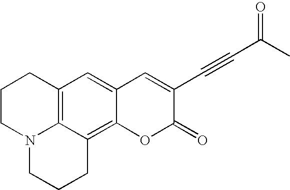 | |
| | C334 |
| 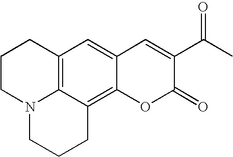 | |

| | |
|---|---|
| | VB204 |
| 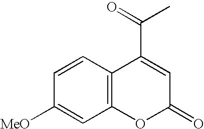 | |
| | VB199 |
| 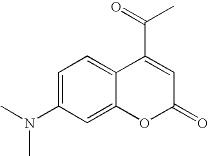 | |

| | |
|---|---|
| | VB45 |
| 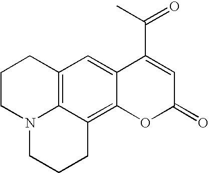 | |

| | |
|---|---|
| | VB243 |
| 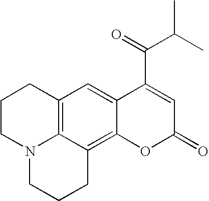 | |
| | VB262 |
| 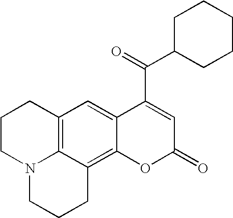 | |
| | VB257 |
| 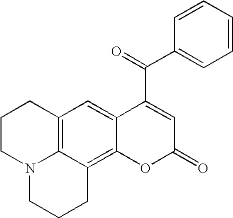 | |
| | VB299 |
| 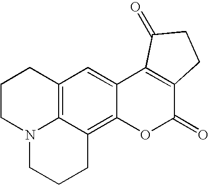 | |
| | VB275 |
| 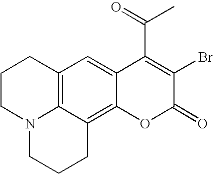 | |
| | VB285 |
| 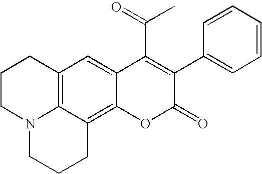 | |
| | VB274 |
| 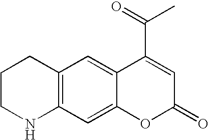 | |
| | VB283 |
| 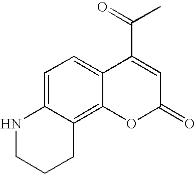 | |

| | |
|---|---|
| | MONAL62 |
| 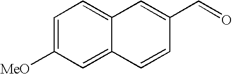 | |

| | |
|---|---|
| | VB463 |
| 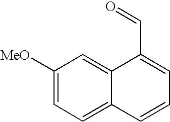 | |
| | VB471 |
| 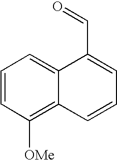 | |

| | |
|---|---|
| | MF-2-91 |
| 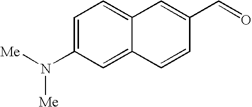 | |

| | |
|---|---|
| | VB417 |
| 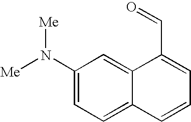 | |
| | VB455 |
| 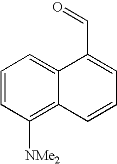 | |
| | VB422 |
| 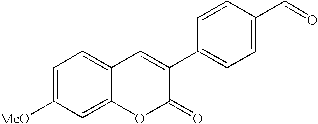 | |
| | VB396 |
| 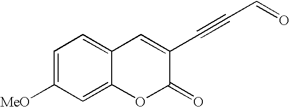 | |
| | VB425 |
| 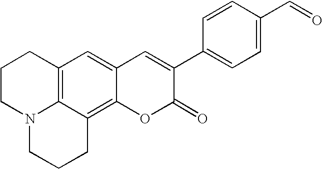 | |
| | VB430 |
| 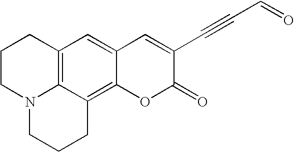 | |
| | VB431 |
| 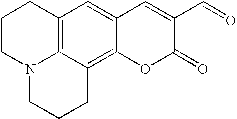 | |

TABLE 7
Photophysical properties of compounds in Table 6.

| | | Fluorescent | | |
|---|---|---|---|---|
| | | compound: | Excitation | Emission |
| COMPOUND: | | Substrate/ | wavelength | wavelength |
| Substrate | Product | Product | (nm) | (nm) |
| MK62 | VB440 | S | 312 | 441 |
| VB468 | VB467 | P (S) | 280 (342) | 354 (473) |
| VB476 | VB475 | P | 294 | 369 |
| DY111 | DY511 | P (S) | 337 (362) | 432 (524) |
| VB412 | VB413 | P | 340 | 427 |
| VB460 | VB459 | P | 300 | 444 |
| DYX1 | VB70 | P | 313 | 440 |
| VB11 | VB12 | P | 342 | 429 |
| VB14 | VB53 | P | 346 | 420 |
| VB35 | VB36 | P | 422 | 509 |
| VB40 | VB42 | P | 429 | 508 |
| C334 | VB93 | P (S) | 400 (465) | 503 (505) |
| VB204 | VB206 | P | 323 | 395 |
| VB199 | VB200 | P | 402 | 489 |
| VB45 | VB47 | P | 398 | 509 |
| VB243 | VB242 | P | 406 | 503 |
| VB262 | VB263 | P | 415 | 502 |
| VB257 | VB261 | P | 409 | 507 |
| VB299 | VB300 | P | 400 | 508 |
| VB275 | VB287 | P | 427 | 524 |
| VB285 | VB286 | P | 409 | 524 |
| VB274 | VB277 | P | 376 | 482 |
| V3283 | VB284 | P | 371 | 490 |
| MONAL62 | VB439 | S (P) | 314 (329) | 451 (350) |
| VB463 | VB464 | S (P) | 352 (282) | 481 (354) |
| VB471 | VB472 | P | 296 | 373 |
| MF-2-91 | VB427 | P (S) | 341 (376) | 432 (527) |
| VB417 | VB418 | P | 340 | 431 |
| VB455 | VB456 | P | 302 | 456 |
| VB422 | VB423 | P | 340 | 428 |
| VB396 | VB395 | P | 351 | 415 |
| VB425 | VB426 | P | 423 | 511 |
| VB430 | VB434 | P | 445 | 505 |
| VB431 | VB432 | S (P) | 465 (402) | 510 (498) |

Substrates for Monitoring Oxidase Activity (Via Oxidation of Aldehydes to Carboxylic Acids)

Initially a product calibration curve is made by plotting fluorescence against varying concentrations of aldehyde and carboxylic acid under normal assay conditions.

When monitoring product formation, one may follow the increase in fluorescence arising from the carboxylic acid by exciting at their respective absorption maxima and monitoring at their respective emission maxima. In the case that the carboxylic acid is the less fluorescent of the two compounds (MONAL62/MA62 and VB237/VB302), it is more favorable to detect enzymatic oxidation by a decrease in fluorescence.

Detecting oxidation of substrate VB463 is done by exciting at 331 nm and monitoring increase in fluorescence at 391 nm. Alternatively, one may monitor the decrease in fluorescence from the substrate VB463 by exciting at 352 nm and monitoring the decrease in fluorescence at 481 nm. This alternative is available for MONAL62/MA62, VB463/VB466 and VB431/Coumarin343.

The emission spectra details for all aldehydes and carboxylic acids are provided in Table 9. All molecules are soluble in DMSO, methanol, and acetonitrile at concentrations of 2.5 mM. The fluorescence spectra are pH independent in the range of 5-9. The molecules are stable in common biological buffers used (Tris-HCl, sodium and potassium phosphate buffers).

TABLE 8
Probes suitable for monitoring oxidase activity.

| | |
|---|---|
| | MONAL62 |
| 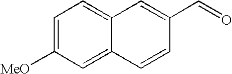 | |

| | |
|---|---|
| | VB463 |
| 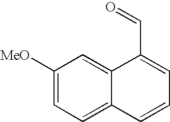 | |
| | VB471 |
| 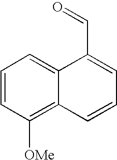 | |

| | |
|---|---|
| | MF-2-91 |
| 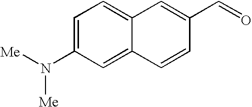 | |

| | |
|---|---|
| | VB417 |
| 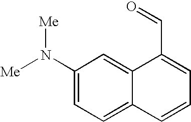 | |
| | VB422 |
| 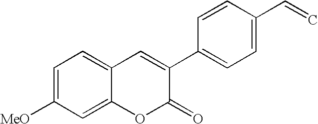 | |
| | VB396 |
| 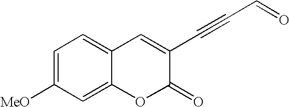 | |
| | VB425 |
| 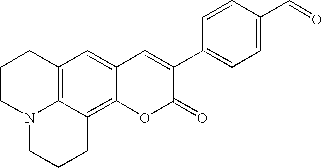 | |
| | VB431 |
| 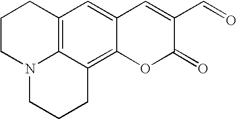 | |
| | VB237 |
| 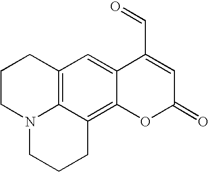 | |

TABLE 9
Photophysical properties of compounds in Table 8.

| | | Fluorescent | | |
|---|---|---|---|---|
| | | compound: | Excitation | Emission |
| COMPOUND: | | Substrate/ | wavelength | wavelength |
| Substrate | Product | Product | (nm) | (nm) |
| MONAL62 | MA62 | S (P) | 314 (292) | 451 (363) |
| VB463 | VB466 | S (P) | 352 (331) | 481 (391) |
| VB471 | VB474 | P | 298 | 439 |
| MF-2-91 | MF-2-53 | P | 322 | 430 |
| VB417 | VB416 | P | 348 | 495 |
| VB422 | VB421 | P | 326 | 431 |
| VB396 | VB438 | P | 359 | 417 |
| VB425 | VB424 | P | 415 | 510 |
| VB431 | C343 | S (P) | 465 (433) | 510 (485) |
| VB237 | VB302 | S | 412 | 513 |

Fluorescence Spectra

All fluorescence emission spectra were recorded with 10 μM solutions of the respective compounds dissolved in DMSO (<2% v/v) in phosphate buffers adjusted to various pHs (5-9). Shown bellow are the spectra at pH=7 read from the wells of a 96-well black plate. All compounds were excited at their respective absorption maxima. Instrument parameters: HV 750, Slits 10.

TABLE 10
Photophysical Properties and Amounts of
All Fluorogenic Substrates and Products:

| | | | | | | Amount |
|---|---|---|---|---|---|---|
| Compound | Structure | λ_{abs} (nm) | ε (M⁻¹cm⁻¹) | λₑₘ (nm) | Φ | (mg) |
| MK62 | 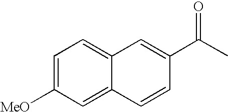 | 312 | 13,200 ± 700 | 441 | N.M. | 32.9 |
| VB440 | 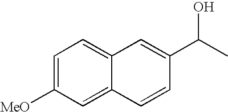 | 330 | 2,000 ± 200 | none | N.M. | 11.8 |
| VB468 | 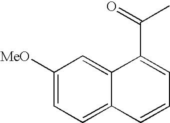 | 342 | 4,900 ± 600 | 473 | 0.0071 ± 0.0002^{a} | 30.1 |
| VB467 | 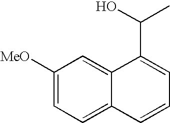 | 280 | 5,700 ± 200 | 354 | N.M. | 15.4 |
| VB476 | 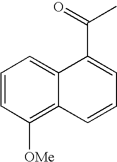 | 326 | 4,400 ± 200 | 532 | 0.00070 ± 0.00007^{a} | 32.0 |
| VB475 | 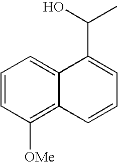 | 294 | 6,800 ± 300 | 369 | N.M. | 22.5 |
| DY111 | 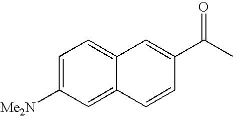 | 362 | 15,500 ± 900 | 524 | 0.139 ± 0.007^{b} | 23.9 |
| DY511 | 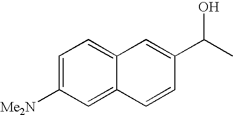 | 337 | 1,550 ± 80 | 432 | 0.59 ± 0.02^{b} | 7.4 |
| VB412 | 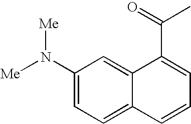 | 383 | 5,500 ± 700 | none | N.M. | 10.4 |
| VB413 | 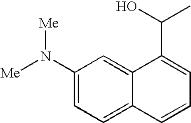 | 340 | 900 ± 100 | 427 | 0.62 ± 0.06^{a} | 11.2 |
| VB460 | 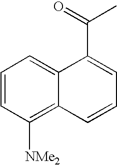 | 330 | 16,800 ± 300 | none | 0.000032 ± 0.000005^{a} | 14.2 |
| VB459 | 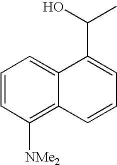 | 300 | 25,700 ± 500 | 444 | N.M. | 12.6 |
| DYX1 | 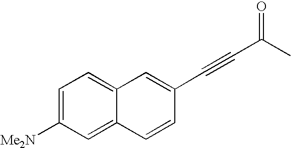 | 372 | 8,400 ± 800 | 470 | 0.0023 ± 0.0001^{b} | 6.5 |
| VB70 | 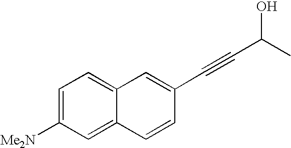 | 313 | 17,000 ± 2000 | 440 | 0.41 ± 0.05^{a} | 5.4 |
| VB11 | 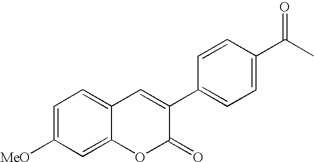 | 317 | 9,600 ± 700 | 462 | 0.041 ± 0.001^{a} | 25.8 |
| VB12 | 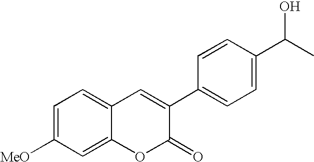 | 342 | 15,300 ± 3000 | 429 | 0.54 ± 0.09^{b} | 5.2 |
| VB14 | 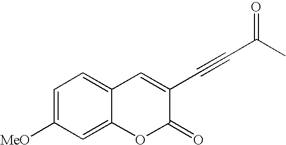 | 368 | 25,000 ± 1000 | 416 | 0.005 ± 0.001^{b} | 4.9 |
| VB53 | 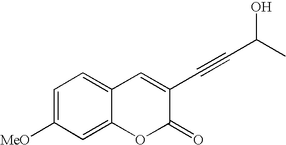 | 346 | 20,300 ± 600 | 420 | 0.79 ± 0.08^{b} | 5.9 |
| VB35 | 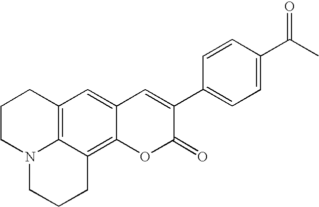 | 435 | 18,400 ± 900 | 511 | 0.0003 ± 0.0001^{c} | 13.8 |
| VB36 | 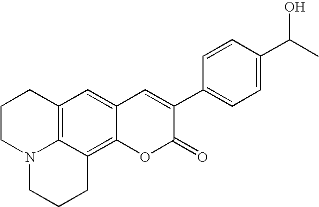 | 422 | 13,400 ± 500 | 509 | 0.36 ± 0.07^{c} | 3.7 |
| VB40 | 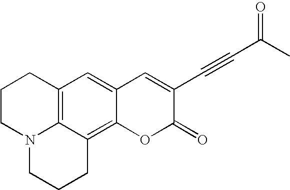 | 464 | 33,000 ± 3000 | 512 | 0.009 ± 0.001^{c} | 4.6 |
| VB42 | 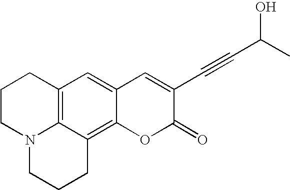 | 429 | 24,000 ± 500 | 508 | 0.50 ± 0.08^{c} | 3.5 |
| C334 | 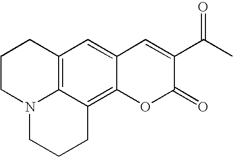 | 465 | 43,000 ± 9,000 | 505 | N.M. | 28.6 |
| VB93 | 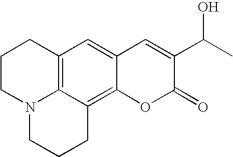 | 344 | 23,600 ± 600 | 503 | N.M. | 5.5 |
| VB204 | 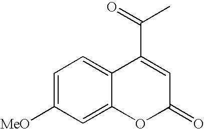 | 341 | 9,100 ± 100 | 404 | 0.028 ± 0.002^{a} | 11.8 |
| VB206 | 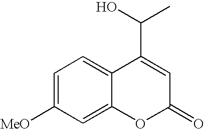 | 323 | 13,400 ± 400 | 395 | 0.65 ± 0.04^{a} | 5.8 |
| VB199 | 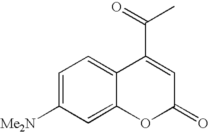 | 436 | 11,500 ± 200 | 501 | 0.0054 ± 0.0008^{c} | 18.4 |
| VB200 | 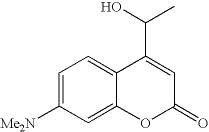 | 402 | 16,100 ± 300 | 489 | 0.15 ± 0.03^{c} | 6.0 |
| VB45 | 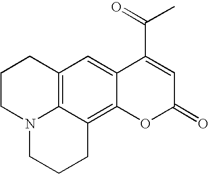 | 436 | 12,900 ± 200 | 520 | 0.00080 ± 0.00008^{c} | 9.8 |
| VB47 | 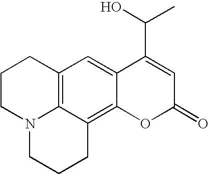 | 398 | 14,500 ± 400 | 509 | 0.29 ± 0.04^{c} | 4.9 |
| VB243 | 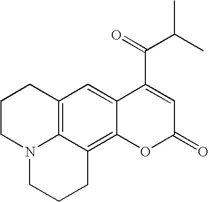 | 431 | 8,500 ± 500 | 514 | 0.0012 ± 0.0002^{c} | 5.4 |
| VB242 | 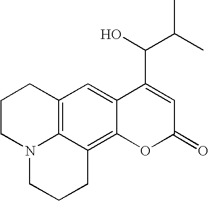 | 406 | 11,300 ± 600 | 503 | 0.59 ± 0.03^{c} | 4.3 |
| VB262 | 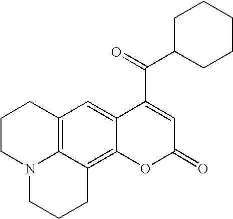 | 440 | 5,200 ± 700 | 598 | 0.0015 ± 0.0001^{c} | 9.9 |
| VB263 | 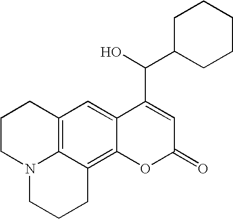 | 415 | 7,200 ± 300 | 502 | 0.26 ± 0.05^{c} | 5.9 |
| VB257 | 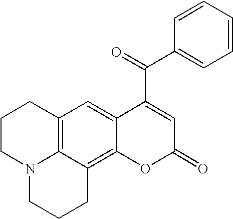 | 427 | 11,200 ± 300 | 608 | 0.0014 ± 0.0002^{c} | 12.7 |
| VB261 | 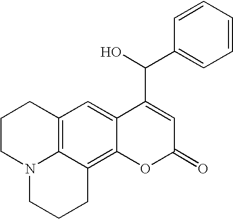 | 409 | 12,500 ± 100 | 507 | 0.32 ± 0.06^{c} | 5.1 |
| VB299 | 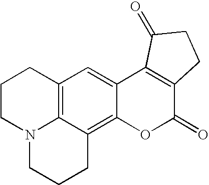 | 464 | 3,700 ± 100 | none | 0.0038 ± 0.0002^{c} | 11.8 |
| VB300 | 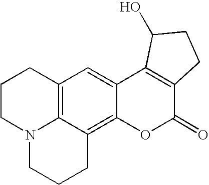 | 400 | 12,300 ± 100 | 508 | 0.43 ± 0.08^{c} | 5.2 |
| VB275 | 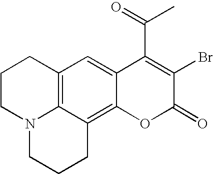 | 442 | 19,000 ± 1,000 | none | 0.0010 ± 0.0003^{c} | 14.6 |
| VB287 | 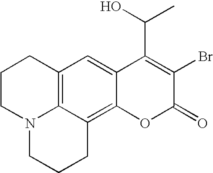 | 427 | 16,700 ± 600 | 524 | 0.05 ± 0.01^{c} | 5.3 |
| VB285 | 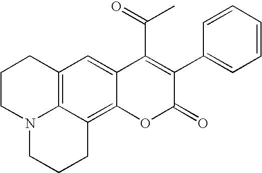 | 437 | 17,000 ± 700 | 595 | 0.0010 ± 0.0002^{c} | 15.8 |
| VB286 | 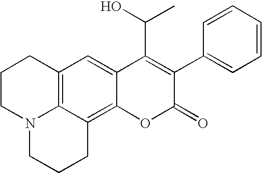 | 409 | 15,000 ± 400 | 524 | 0.12 ± 0.02^{c} | 6.2 |
| VB274 | 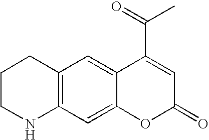 | 409 | 11,500 ± 300 | 487 | 0.0133 ± 0.0001^{c} | 10.2 |
| VB277 | 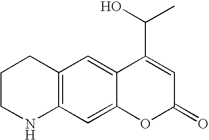 | 376 | 14,900 ± 400 | 482 | 0.72 ± 0.10^{b} | 5.2 |
| VB283 | 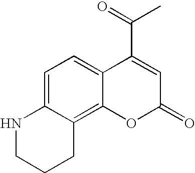 | 404 | 9,300 ± 100 | 497 | 0.0054 ± 0.0004^{c} | 12.3 |
| VB284 | 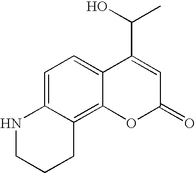 | 371 | 13,300 ± 200 | 490 | 0.49 ± 0.10^{b} | 6.1 |
| MONAL62 | 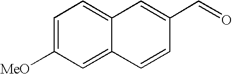 | 314 | 18,000 ± 2,000 | 451 | 0.25 ± 0.06^{a} | 45.5 |
| VB439 | 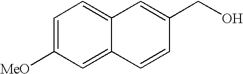 | 329 | 4,600 ± 300 | 350 | 0.118 ± 0.003^{a} | 12.2 |
| MA62 | 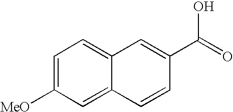 | 292 | 8.300 ± 300 | 363 | N.M. | 26.6 |
| VB463 | 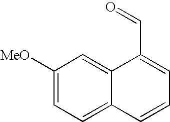 | 352 | 6,300 ± 400 | 481 | 0.0186 ± 0.0006^{a} | 27.0 |
| VB464 | 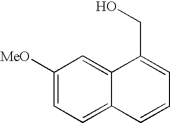 | 282 | 4,400 ± 400 | 354 | N.M. | 9.8 |
| VB466 | 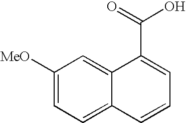 | 331 | 2,900 ± 300 | 391 | 0.0184 ± 0.0006^{a} | 17.5 |
| VB471 | 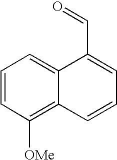 | 354 | 21,000 ± 2,000 | 540 | 0.00056 ± 0.00008^{a} | 37.5 |
| VB472 | 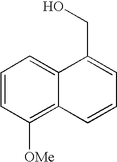 | 296 | 7,000 ± 900 | 373 | N.M. | 10.0 |
| VB474 | 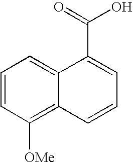 | 298 | 6,500 ± 900 | 439 | N.M. | 17.8 |
| MF-2-91 | 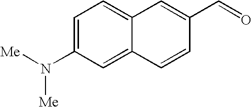 | 376 | 17,000 ± 1000 | 527 | 0.021 ± 0.004^{a} | 16.8 |
| VB427 | 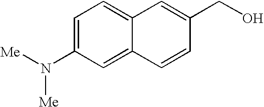 | 341 | 650 ± 80 | 432 | 0.368 ± 0.001^{a} | 5.3 |
| MF-2-53 | 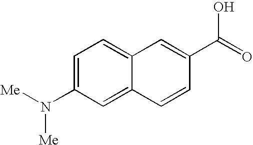 | 322 | 17,700 ± 300 | 430 | 0.77 ± 0.01^{a} | 7.5 |
| VB417 | 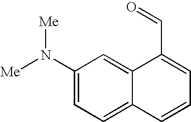 | 421 | 5,000 ± 700 | none | 0.00092 ± 0.00003^{a} | 15.7 |
| VB418 | 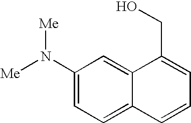 | 340 | 1,300 ± 200 | 431 | 0.90 ± 0.07^{a} | 9.2 |
| VB416 | 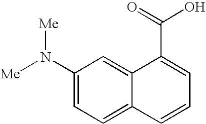 | 348 | 6,000 ± 200 | 495 | 1.00 ± 0.03^{a} | 6.1 |
| VB455 | 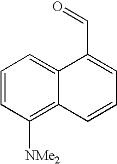 | 354 | 3,570 ± 0 | 449 | N.M. | 14.9 |
| VB456 | 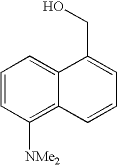 | 302 | 6,100 ± 500 | 456 | N.M. | 19.2 |
| VB422 | 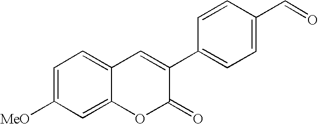 | 347 | 12,000 ± 1,000 | 477 | 0.015 ± 0.008^{a} | 12.8 |
| VB423 | 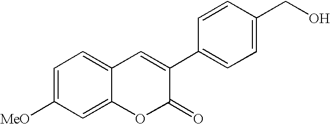 | 340 | 18,400 ± 600 | 428 | 0.95 ± 0.06^{a} | 8.8 |
| VB421 | 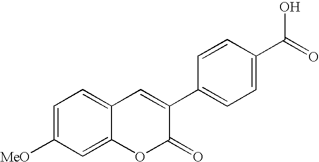 | 326 | 9,000 ± 1,000 | 431 | 1.003 ± 0.007^{a} | 3.8 |
| VB396 | 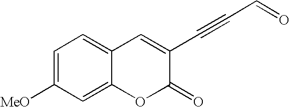 | 358 | 24,000 ± 1,000 | 414 | 0.29 ± 0.04^{a} | 19.8 |
| VB395 | 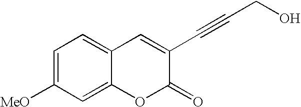 | 351 | 20,000 ± 2,000 | 415 | 0.071 ± 0.005^{a} | 5.3 |
| VB438 | 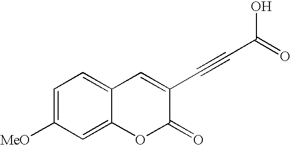 | 359 | 26,000 ± 1,000 | 417 | 1.1 ± 0.1^{a} | 4.0 |
| VB425 | 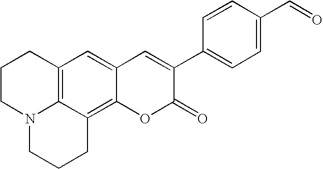 | 443 | 30,000 ± 5,000 | 509 | 0.000044 ± 0.000005^{a} | 13.1 |
| VB426 | 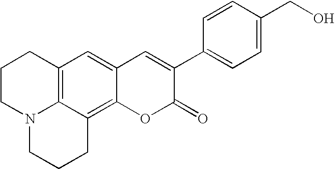 | 423 | 16,000 ± 2,000 | 511 | 0.0057 ± 0.0008^{a} | 3.7 |
| VB424 | 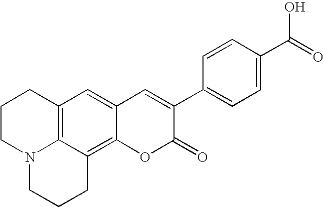 | 415 | 13,000 ± 2,000 | 510 | 0.0048 ± 0.0002^{a} | 7.4 |
| VB430 | 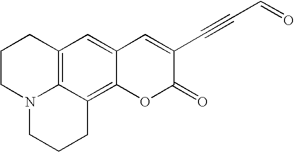 | 455 | 21,000 ± 2,000 | 502 | 0.00093 ± 0.00003^{a} | 9.4 |
| VB434 | 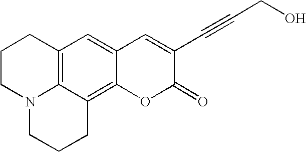 | 445 | 24,000 ± 1,000 | 505 | 0.0127 ± 0.0002^{a} | 3.6 |
| VB431 | 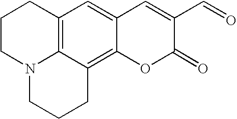 | 465 | 44,000 ± 6,000 | 510 | 0.0083 ± 0.0002^{a} | 15.2 |
| VB432 | 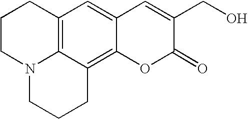 | 402 | 16,800 ± 200 | 498 | 0.0095 ± 0.0000^{a} | 5.4 |
| C343 | 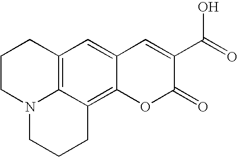 | 433 | 16,000 ± 2,000 | 485 | 0.043 ± 0.000^{a} | 17.9 |
| VB237 | 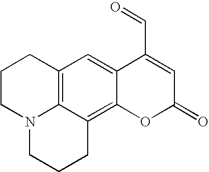 | 412 | 10,000 ± 1,000 | 513 | 0.41 ± 0.05^{c} | 8.3 |
| VB302 | 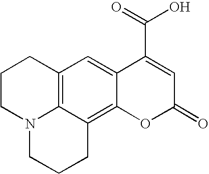 | 411 | 16,000 | 580 | 0.000013 ± 0.000002^{a} | 6.7 |

Extinction coefficients (ε) reported are the average of triplicate measurements of the lowest energy wavelength transition at three different concentrations.
Fluorescence quantum yields (Φ) are the average of three independent quantum yield determinations and are determined by excitation at 340, 365, or 420 nm using either 9,10-diphenylanthracene in EtOH (excited at 340 nm^{a} or 365 nm^{b}) or coumarin 6 in EtOH (excited at 420 nm^{c}).
All measurements performed at pH 7 doubly deionized water (4% acetonitrile).

Cell Culture Experiments

HepG2 cells were obtained from and grown in the Penning laboratory (University of Pennsylvania School of Medicine). Cells were maintained at 37° C. and 5% CO₂ containing Eagle's minimal essential medium supplemented with 100 U/mL penicillin, 100 μg/mL streptomycin, 2 mM L-glutamine, and 10 % heat-inactivated fetal bovine serum.

To measure metabolism of 5c in HepG2 cell fractions, the cells were harvested and fractionized as follows. HepG2 cells were grown to confluency on eight 15×100 mm dishes, whereupon 500 μL of ice cold Tris-HCl-sucrose buffer (50 mM Tris-HCl at pH 7.4, 250 mM sucrose, 1 mM EDTA, and 1 mM 2-mercaptoethanol) was added to each dish. Cells were scratched off and taken up directly into an ice cold potter. These cells were then homogenized and sonicated (10 one-second 10 W bursts, four times on ice). The cells were transferred to a 15 mL FALCON tube and centrifuged at 800g for 10 minutes at 4° C. to remove cellular debris. Aliquots of the resultant supernatant were taken up and stored with glycerol (30%) at −78° C. The rest of the supernatant was centrifuged at 100,000 g for 1 hour at 4° C. to obtain the cytosolic fraction (supernatant). The cytosolic fractions were similarly stored at −78° C. with 30% glycerol prior to usage. The remaining pellet (microsomes) were washed with Tris-HCl-sucrose buffer and redissolved in a volume of Tris-HCl-sucrose buffer equivalent to that of the cytosolic fraction. The microsomes were rehomogenized in a potter, sonicated, and recentrifuged for 1 hour at 100,000 g and 4° C. The resultant supernatant was discarded. The pellet of microsomes was redissolved in a volume of Tris-HCl-sucrose buffer equivalent to that of the cytosolic fraction. After homogenization, the microsomes were stored at −78° C. with glycerol (30%) until usage.

Metabolism of Phenyl Ketone 5c in Cellular Lysates

Protein concentrations for whole or fractionized HepG2 cells were determined by standard Bradford assays (Bradford, M. M.; Anal. Biochem. 1976, 72, 248). To determine metabolism of 5c in cellular lysates, 10 μM of fluorogenic substrate was incubated in 1 mL assay volumes of 1 mM NADPH, 50 mM Tris-HCl at pH 7.4, 250 mM sucrose, 1 mM EDTA, 1 mM 2-mercaptoethanol, and 5 mM MgCl₂. For HepG2 assays with inhibitor, the assay mixture was also preincubated with 100 μM flufenamic acid. Reactions were initiated with 80 μg of protein per 1 mL assay and monitored fluorimetrically for up to 2 hours. Product formation was approximated as described above (equation 1).

1. A compound of the structure: 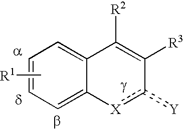
wherein
Y is O, X is O, and bond y is a single bond, or
Y is absent, X is CH and bond γ is a double bond,
wherein R¹ is bound at carbon δ and is —H, —OH, —O-alkyl, —NH-alkyl, —N(alkyl)₂, —NH₂, aryl, heteroaryl, -alkyl-C(O) (OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl-C(O)H, -alkynyl-CH₂OH, or -alkynyl-C(O)OH; or
R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,
or R¹ is bound to carbon α and is —N(alkyl)₂, R² is —C(O)H, —CH₂OH, —C(O)OH, —C(O)CH₃, —CH(OH)CH₃, and R³ is H,
or R¹ is bound to carbon α and is —O-alkyl, R² is —CH(OH)CH₃ or —C(O)OH, and R³ is H,
or R¹ is bound to carbon β and is —O-alkyl, R² is —C(O)H, —C(O)OH, —CH₂OH, —C(O)CH₃, —CH(OH)CH₃, and R³ is H,
or R¹ is bound to carbon β and is —N(alkyl)₂, R² is —C(O)H, —C(O)OH, —CH₂OH, —C(O)CH₃, —CH(OH)CH₃, and R³ is H,
or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl-C(O)H, -alkynyl-CH₂OH, —C(O)R⁷, —CH(OH)R⁸, —R¹⁰—C(O)R⁹, —R¹⁰—CH(OH)R⁹, —C(CX₂) (aryl) where X is a halide, —C(CX₂) (alkyl) where —X is a halide, —C(CHX) (aryl) where X is a halide, —C(═NOH) (aryl), —CH(CH₃) (aryl), —CH₂— (aryl), or —C(CH₂) (aryl);
or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX) (aryl) where X is a halide, —C(═NOH) (aryl), —CH(CH₃) (aryl), —CH₂— (aryl), or —C(CH₂) (aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl,
wherein when R¹ is —N(CH₃)₂ and is bound at carbon δ and R³ is —C(O)CH₃ or —CH(OH) (CH₃), or R¹ is —O-alkyl and is bound at carbon δ and R³ is —C(O)H, then R² is OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴, Y is O, X is O and bond γ is a single bond,
or a salt or stereoisomer thereof.

2. The compound of claim 1, wherein when R¹ is —O—CH₃ and bound at carbon δ and R³ is —C(O)H, —C(O)CH₃ or —CH(OH)(CH₃), Y is absent, X is CH and bond γ is a double bond, then R² is OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R^{4′}, —R⁵—C(O)R⁴, or —R⁵—CH(OH)R⁴, where R^{4,} is ethyl, alkenyl, alkynyl, substituted aryl or unsubstituted aryl.

3-7. (canceled)

8. The compound of claim 1, having the structure: 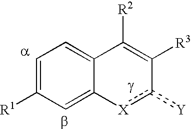
wherein
Y is O, X is O, and bond y is a single bond, or
Y is absent, X is CH and bond y is a double bond,
wherein R¹ is —H, —OH, —O-alkyl, —NH-alkyl, —N(alkyl)₂, —NH₂, aryl, heteroaryl, -alkyl-C(O) (OH), -alkyl-OH, or R¹ is >NH which is covalently bound to carbon α or to carbon β; R² is H, OH, a C₂-C₇ alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, or —R⁵—CH(OH)R⁴; or R² and R³ together form a ring substituted with ═O,
where R⁴ is methyl, alkenyl, alkynyl, or aryl; R⁵ is alkyl, alkenyl, alkynyl, aryl, or cycloalkyl; and R⁶ is alkenyl, alkynyl, aryl, or cycloalkyl,
or R¹ is —N< which is covalently bound to both carbon α and carbon β and either R² is —H and R³ is —C(O)R⁷, —CH(OH)R⁸, —R¹⁰—C(O)R⁹, —R¹⁰—CH(OH)R⁹, —C(CX₂) (aryl) where X is a halide, —C(CX₂)(alkyl) where X is a halide, —C(CHX)(aryl) where X is a halide, —C(═NOH) (aryl), —CH(CH₃) (aryl), —CH₂— (aryl), or —C(CH₂) (aryl); or R³ is —H and R² is —C(O)R¹¹, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃)(aryl), —CH₂— (aryl), or —C(CH₂)(aryl); or R² and R³ together form a ring substituted with ═O,
where R⁷ is cycloalkyl, C₂-C₇ alkyl, alkenyl, alkynyl, aryl, or heteroaryl, R⁸ is hydroxyl, alkyl, cycloalkyl, alkenyl, alkynyl, aryl, or heteroaryl, R⁹ is alkyl, cycloalkyl, alkenyl, alkynyl, aryl, or heteroaryl, R¹⁰ is alkynyl, aryl, or heteroaryl, and R¹¹ is methyl, hydroxyl, alkenyl, alkynyl, aryl, or heteroaryl,
wherein when R¹ is —N(CH₃)₂ and R³ is —C(O)CH₃ or —CH(OH) (CH₃), then R² is OH, a C₂-C₇ alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁴, —CH(OH)R⁴, —R⁵—C(O)R⁴, or —R⁵—CH(OH)R4, Y is O, X is O and bond γ is a single bond,
or a salt or stereoisomer thereof.

9. (canceled)

10. (canceled)

11. The compound of claim 8, having the structure: 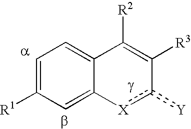
wherein Y is absent, X is CH, and γ is a double bond.

12. (canceled)

13. (canceled)

14. (canceled)

15. (canceled)

16. The compound of claim 1 or 8, having the structure: 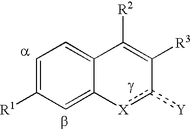
wherein X is O, Y is O, and γ is a single bond,
wherein R¹ is —H, —OH, —O-alkyl, —NH-alkyl, —N(alkyl)₂, —NH₂, aryl, heteroaryl, -alkyl-C(O) (OH), -alkyl-OH, or R¹ is >NH which is covalently bound to carbon α or to carbon β.

17. (canceled)

18. (canceled)

19. (canceled)

20. (canceled)

21. (canceled)

22. (canceled)

23. (canceled)

24. (canceled)

25. The compound of claim 1 or 8, having the structure: 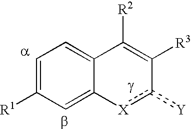
wherein X is O, Y is O, and γ is a single bond,
wherein R¹ is —N< which is covalently bound to both carbon α and carbon β.

26-183. (canceled)

184. The compound of claim 1, having the structure:

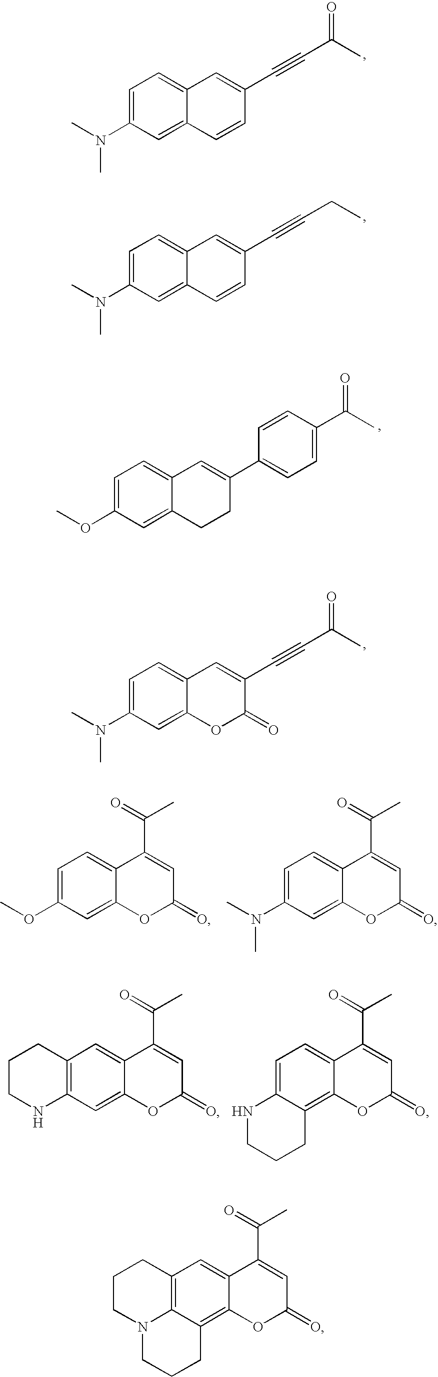

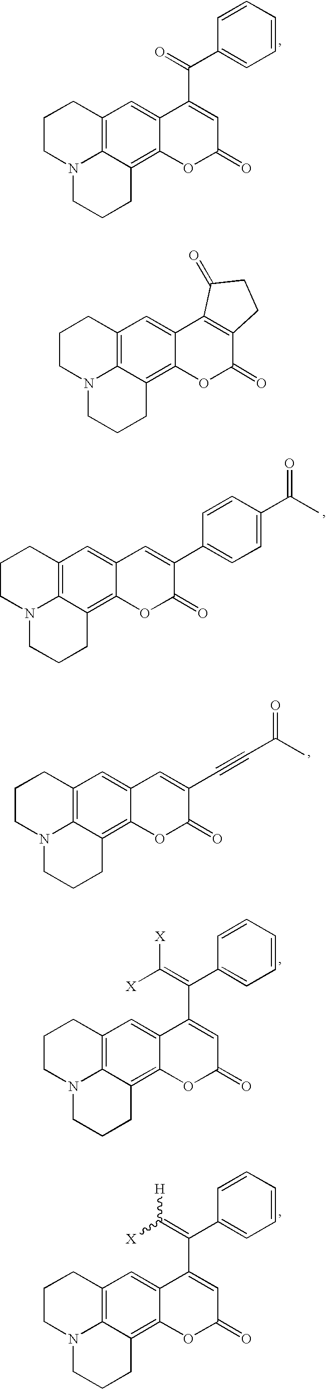

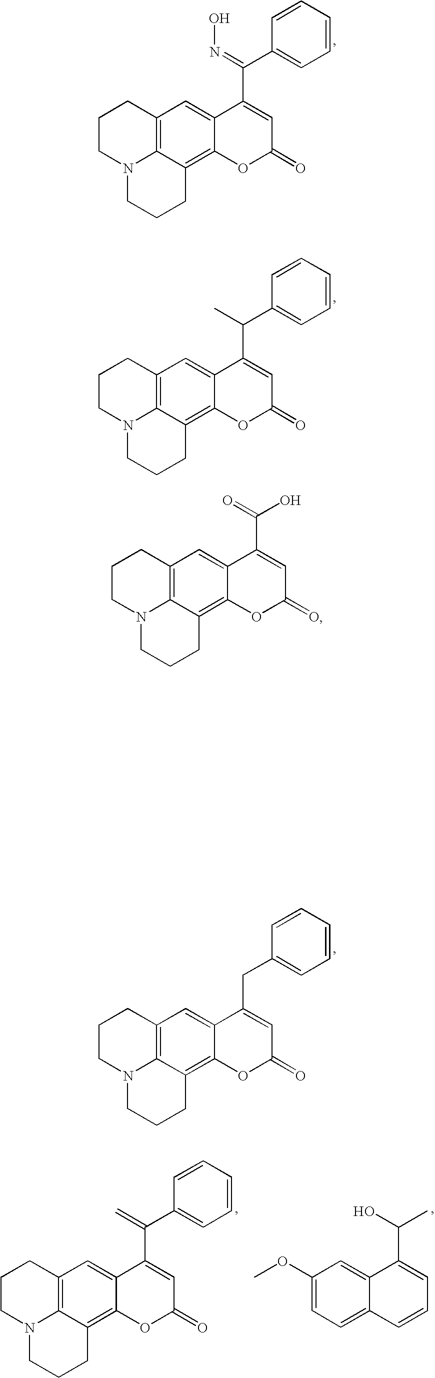

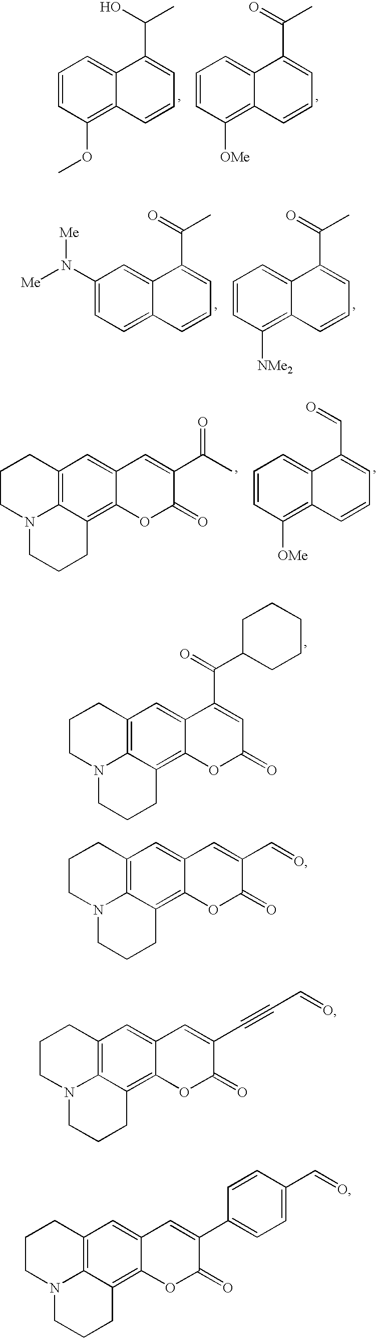

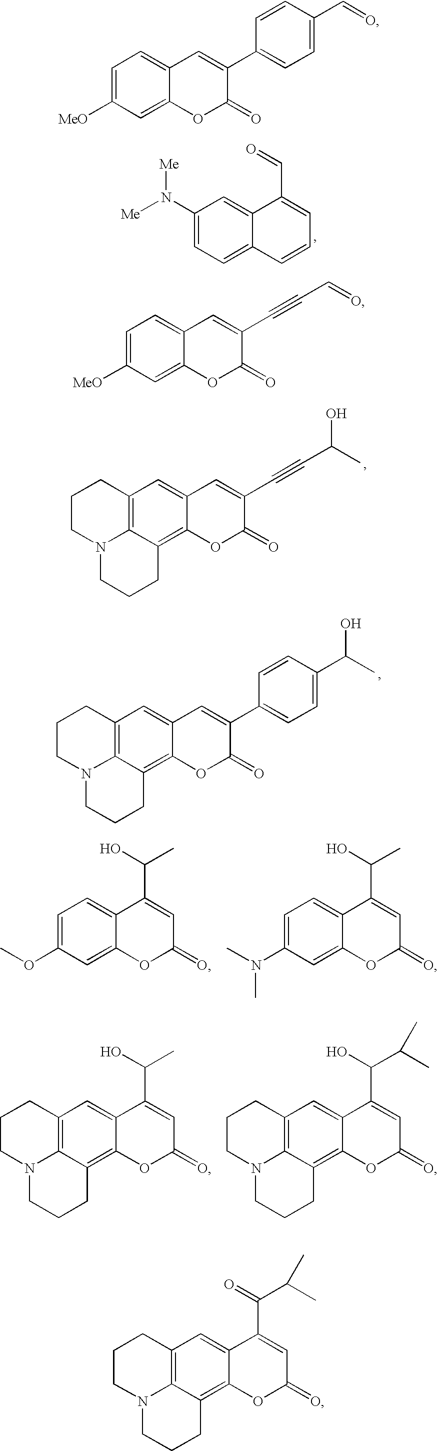

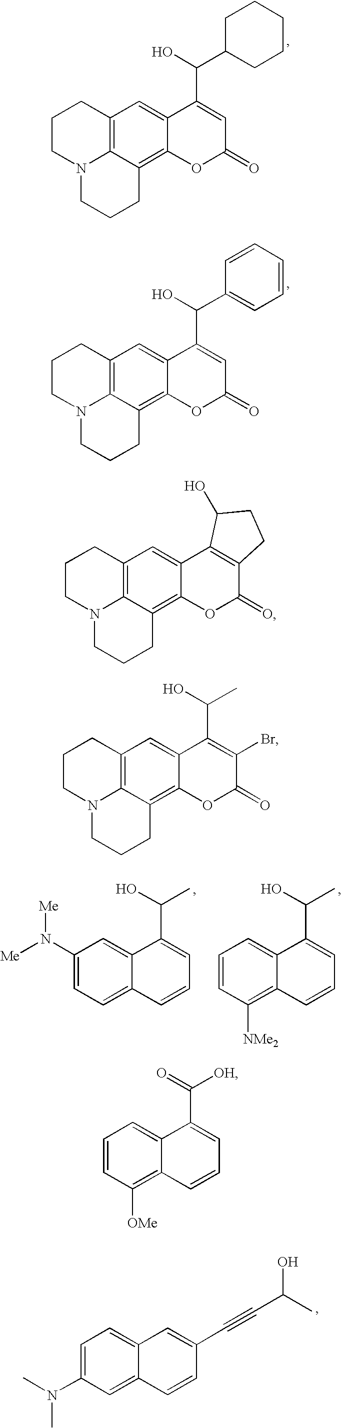

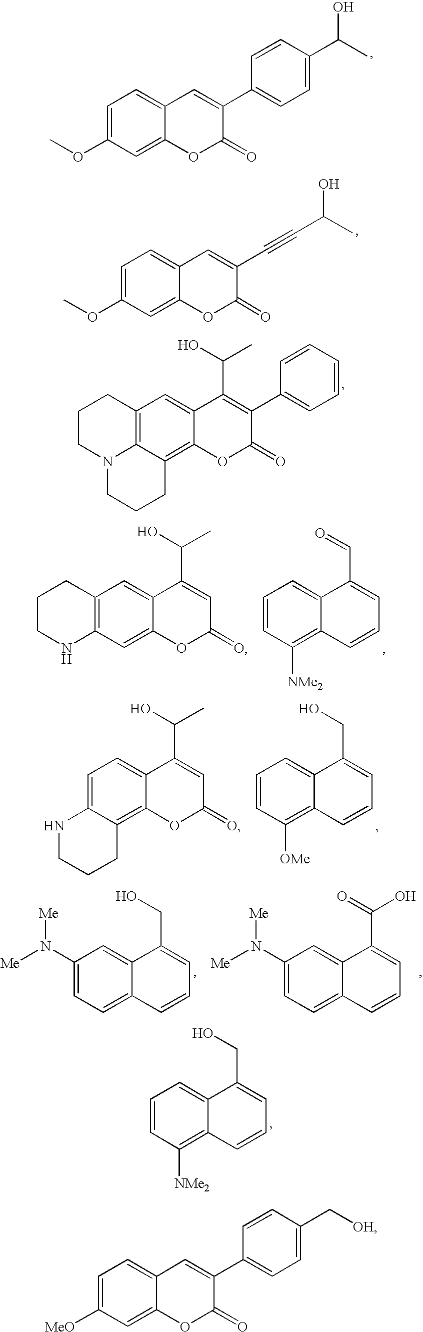

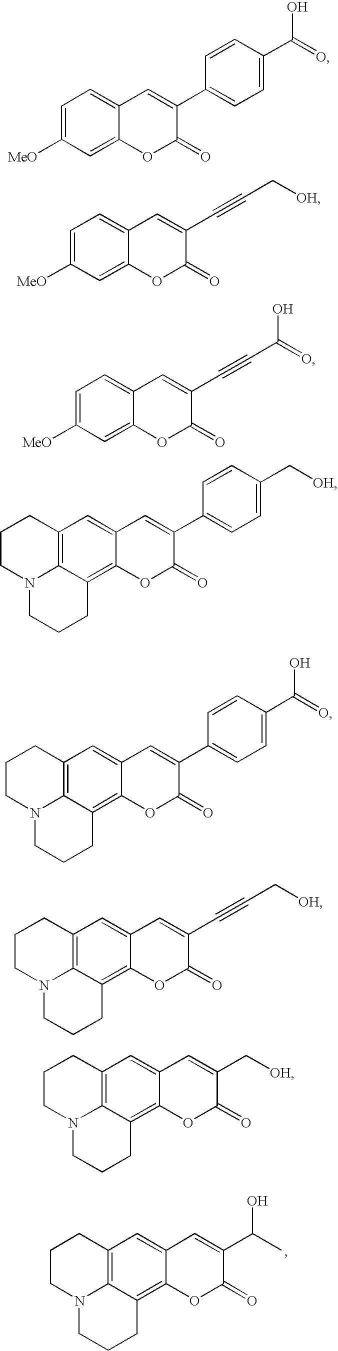

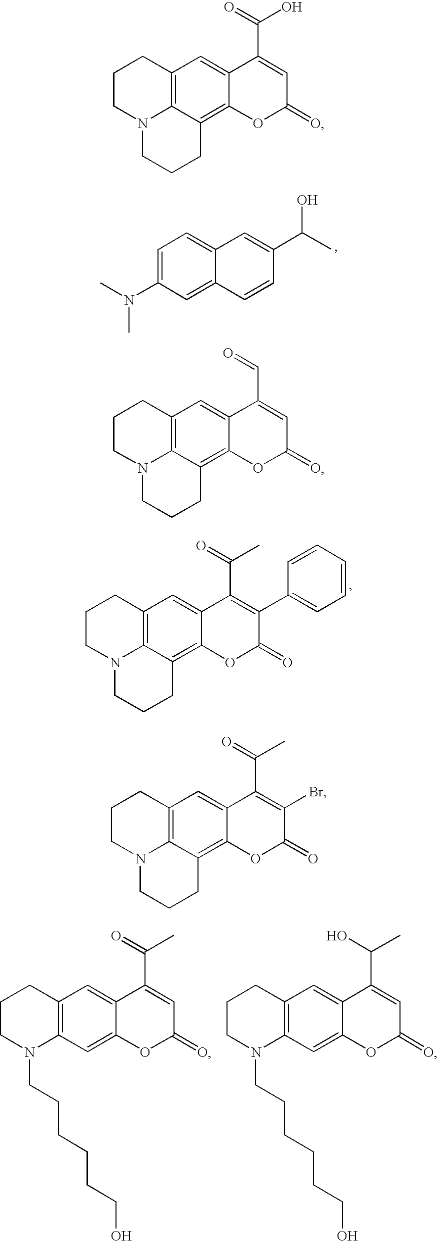

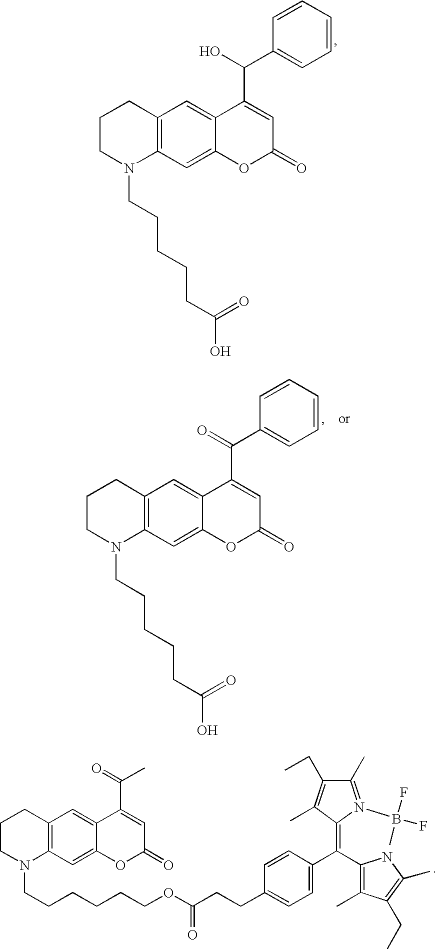

185. A process for preparing the compound of claim 1 comprising:
reacting a compound having the structure: 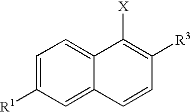
wherein X is —Br, —I, or —OTf with any one of
(i) a compound having the structure: 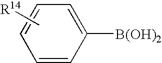
or
(ii) a compound having the structure: 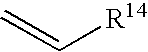
or
(iii) a compound having the structure: 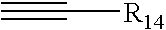
in the presence of palladium of a zero oxidation state to produce a compound having the structure: 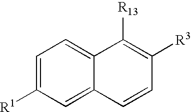
wherein R¹³ is: 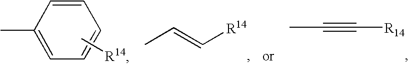
wherein R¹⁴ is any of R² or R³,
wherein R¹ is bound at carbon δ and is —H, —OH, —O-alkyl, —NH-alkyl, —N(alkyl)₂, —NH₂, aryl, heteroaryl, -alkyl-C(O) (OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R , —CH(OH)R⁴, —R⁵—C(O)R⁴, —R⁵—CH(OH)R⁴, -aryl-C(O)H, -aryl-CH₂OH, -aryl-C(O)OH, -alkynyl-C(O)H, -alkynyl-CH₂OH, or -alkynyl-C(O)OH; or
R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,
or R¹ is bound to carbon α and is —N(alkyl)₂, R² is —C(O)H, —CH₂OH, —C(O)OH, —C(O)CH₃, —CH(OH)CH₃, and R³ is H,
or R¹ is bound to carbon α and is —O-alkyl, R² is —CH(OH)CH₃ or —C(O)OH, and R³ is H,
or R¹ is bound to carbon β and is —O-alkyl, R² is —C(O)H, —C(O)OH, —CH₂OH, —C(O)CH₃, —CH(OH)CH₃, and R³ is H,
or R¹ is bound to carbon β and is —N(alkyl)₂, R² is —C(O)H, —C(O)OH, —CH₂OH, —C(O)CH₃, —CH(OH)CH₃, and R³ is H,
or R¹ is bound to carbon 6 and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl-C(O)H, -alkynyl-CH₂OH, —C(O)R⁷, —CH(OH)R⁸, —R¹⁰—C(O)R⁹, —R¹⁰—CH(OH)R⁹, —C(CX₂) (aryl) where X is a halide, —C(CX₂) (alkyl) where —X is a halide, —C(CHX) (aryl) where X is a halide, —C(═NOH) (aryl), —CH(CH₃) (aryl), —CH₂— (aryl), or —C(CH₂) (aryl);
or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂) (aryl) where X is a halide, —C(CHX) (aryl) where X is a halide, —C(═NOH) (aryl), —CH (CH₃) (aryl), —CH₂— (aryl), or —C(CH₂) (aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl, or
reacting a compound having the structure: 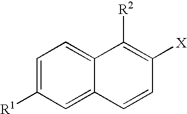
wherein X is —Br, —I, or —OTf
with any one of
(i) a compound having the structure: 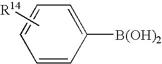
or
(ii) a compound having the structure: 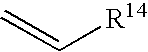
or
(iii) a compound having the structure: 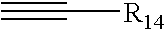
in the presence of palladium of a zero oxidation state to produce a compound having the structure: 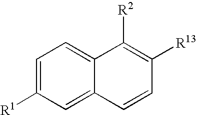
wherein R¹³ is: 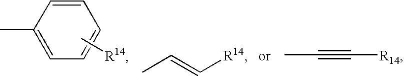
wherein R¹⁴ is any of R²or R³,
wherein R¹ is bound at carbon δ and is —H, —OH, —O-alkyl, —NH-alkyl, —N(alkyl)₂, —NH₂, aryl, heteroaryl, -alkyl-C(O) (OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl-C(O)H, -alkynyl-CH₂OH, or -alkynyl-C(O)OH; or
R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,
or R¹ is bound to carbon α and is —N(alkyl)₂, R² is —C(O)H, —CH₂OH, —C(O)OH, —C(O)CH₃, —CH(OH)CH₃, and R³ is H,
or R¹ is bound to carbon α and is —O-alkyl, R² is —CH(OH)CH₃ or —C(O)OH, and R³ is H,
or R¹ is bound to carbon β and is —O-alkyl, R² is —C(O)H, —C(O)OH, —CH₂OH, —C(O)CH₃, —CH(OH)CH₃, and R³ is H,
or R¹ is bound to carbon β and is —N(alkyl)₂, R² is —C(O)H, —C(O)OH, —CH₂OH, —C(O)CH₃, —CH(OH)CH₃, and R³ is H,
or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl-C(O)H, -alkynyl-CH₂OH, —C(O)R⁷, —CH(OH)R⁸, —R¹⁰—C(O)R⁹, —R¹⁰—CH(OH)R⁹, —C(CX₂) (aryl) where X is a halide, —C(CX₂) (alkyl) where —X is a halide, —C(CHX) (aryl) where X is a halide, —C(═NOH) (aryl), —CH(CH₃) (aryl), —CH₂— (aryl), or —C(CH₂) (aryl);
or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂) (aryl) where X is a halide, —C(CHX) (aryl) where X is a halide, —C(═NOH) (aryl), —CH(CH₃) (aryl), —CH₂— (aryl), or —C(CH₂) (aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl, or
reacting a compound having the structure: 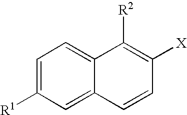
wherein X is —Br, —I, or —OTf
with any one of
(i) a compound having the structure: 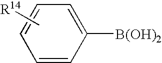
or
(ii) a compound having the structure: 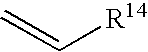
or
(iii) a compound having the structure: 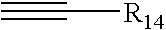
in the presence of palladium of a zero oxidation state to produce a compound having the structure: 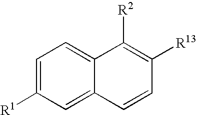
wherein R¹³ is: 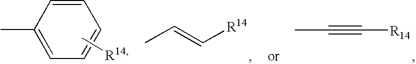
wherein R¹⁴ is any of R² or R³,
wherein R¹ is bound at carbon δ and is —H, —OH, —O-alkyl, —NH-alkyl, —N(alkyl)₂, —NH₂, aryl, heteroaryl, -alkyl-C(O) (OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl-C(O)H, -alkynyl-C(O)OH, or -alkynyl-CH₂OH; or
R² and R³ together form a ring substituted with ═O,
where R⁴ is methyl, ethyl, alkenyl, alkynyl, a substituted aryl or an unsubstituted aryl, R⁵ is alkyl, alkenyl, alkynyl, substituted aryl or an unsubstituted aryl, or cycloalkyl, and R⁶ is hydrogen, methyl, a C₃-C₇ alkyl, alkenyl, alkynyl, aryl, or cycloalkyl,
or R¹ is bound to carbon α and is —N(alkyl)₂, R² is —C(O)H, —CH₂OH, —C(O)OH, —C(O)CH₃, —CH(OH)CH₃, and R³ is H,
or R¹ is bound to carbon α and is —O-alkyl, R² is —CH(OH)CH₃ or —C(O)OH, and R³ is H,
or R¹ is bound to carbon β and is —O-alkyl, R² is —C(O)H, —C(O)OH, —CH₂OH, —C(O)CH₃, —CH(OH)CH₃, and R³ is H,
or R¹ is bound to carbon β and is —N(alkyl)₂, R² is —C(O)H, —C(O)OH, —CH₂OH, —C(O)CH₃, —CH(OH)CH₃, and R³ is H,
or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl-C(O)H, -alkynyl-CH₂OH, —C(O)R⁷, —CH(OH)R⁸, —R¹⁰—C(O)R⁹, —R¹⁰—CH(OH)R⁹, —C(CX₂) (aryl) where X is a halide, —C(CX₂) (alkyl) where X is a halide, —C(CHX) (aryl) where X is a halide, —C(═NOH) (aryl), —CH(CH₃) (aryl), —CH₂— (aryl), or —C(CH₂) (aryl);
or R³ is —H or X where X is a halide, alkyl, alkenyl, alkoxy, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂) (aryl) where X is a halide, —C(CHX) (aryl) where X is a halide, —C(═NOH) (aryl), —CH(CH₃) (aryl), —CH₂— (aryl), or —C(CH₂) (aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl; or heteroaryl, R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl, or
reacting a compound having the structure: 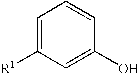
with a compound having the structure: 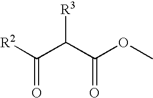
to produce a compound having the structure: 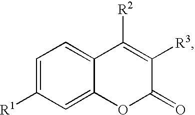
wherein R¹ is bound at carbon δ and is —H, —OH, —O-alkyl, —NH-alkyl, —N(alkyl)₂, —NH₂, aryl, heteroaryl, -alkyl-C(O) (OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, 13 O-alkyl, —O-alkenyl, —O-alkynyl, —O-aryl, —O-cycloalkyl, —NH-alkyl, —N(alkyl)₂, halide, —C(O)R⁴, —CH(OH)R⁴, —R⁵—C(O)R⁴, —R⁵—CH(OH)R⁴, -aryl-C(O)H, -aryl-CH₂OH, -aryl-C(O)OH, -alkynyl-C(O)H, -alkynyl-CH₂OH, or -alkynyl-C(O)OH; or
R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,
or R¹ is bound to carbon α and is —N(alkyl)₂, R² is —C(O)H, —CH₂OH, —C(O)OH, —C(O)CH₃, —CH(OH)CH₃, and R³ is H,
or R¹ is bound to carbon α and is —O-alkyl, R² is —CH(OH)CH₃ or —C(O)OH, and R³ is H,
or R¹ is bound to carbon β and is —O-alkyl, R² is —C(O)H, —C(O)OH, —CH₂OH, —C(O)CH₃, —CH(OH)CH₃, and R³ is H,
or R¹ is bound to carbon β and is —N(alkyl)₂, R² is —C(O)H, —C(O)OH, —CH₂OH, —C(O)CH₃, —CH(OH)CH₃, and R³ is H,
or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl-C(O)H, -alkynyl-CH₂OH, —C(O)R⁷, —CH(OH)R⁸, —R¹⁰—C(O)R⁹, —R¹⁰—CH(OH)R⁹, —C(CX₂) (aryl) where X is a halide, —C(CX₂) (alkyl) where —X is a halide, —C(CHX) (aryl) where X is a halide, —C(═NOH) (aryl), —CH(CH₃) (aryl), —CH₂— (aryl), or —C(CH₂) (aryl);
or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂) (aryl) where X is a halide, —C(CHX) (aryl) where X is a halide, —C(═NOH) (aryl), —CH(CH₃) (aryl), —CH₂— (aryl), or —C(CH₂) (aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl, or
reacting a compound having the structure: 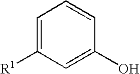
with a compound having the structure: 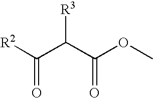
to produce a compound having the structure: 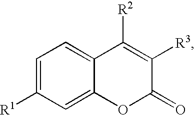
or
reacting a compound having the structure: 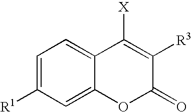
wherein X is —Br, —I, or —OTf
with any one of
(i) a compound having the structure: 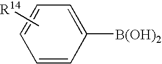
or
(ii) a compound having the structure: 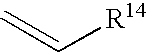
or
(iii) a compound having the structure: 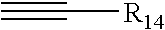
in the presence of palladium of a zero oxidation state to produce a compound having the structure: 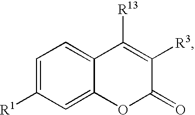
wherein R¹³ is: 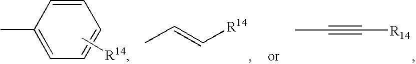
wherein R¹ is bound at carbon δ and is —H, —OH, —O-alkyl, —NH-alkyl, —N(alkyl)₂, —NH₂, aryl, heteroaryl, -alkyl-C(O) (OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl-C(O)H, -alkynyl-CH₂OH, or -alkynyl-C(O)OH; or
R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,
or R¹ is bound to carbon α and is —N(alkyl)₂, R² is —C(O)H, —CH₂OH, —C(O)OH, —C(O)CH₃, —CH(OH)CH₃, and R³ is H,
or R¹ is bound to carbon α and is —O-alkyl, R² is —CH(OH)CH₃ or —C(O)OH, and R³ is H,
or R¹ is bound to carbon β and is —O-alkyl, R² is —C(O)H, —C(O)OH, —CH₂OH, —C(O)CH₃, —CH(OH)CH₃, and R³ is H,
or R¹ is bound to carbon β and is —N(alkyl)₂, R² is —C(O)H, —C(O)OH, —CH₂OH, —C(O)CH₃, —CH(OH)CH₃, and R³ is H,
or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl-C(O)H, -alkynyl-CH₂OH, —C(O)R⁷, —CH(OH)R⁸, —R¹⁰—C(O)R⁹, —R¹⁰—CH(OH)R⁹, —C(CX₂) (aryl) where X is a halide, —C(CX₂) (alkyl) where —X is a halide, —C(CHX) (aryl) where X is a halide, —C(═NOH)(aryl), —CH(CH₃) (aryl), —CH₂— (aryl), or —C(CH₂) (aryl);
or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX) (aryl) where X is a halide, —C(═NOH) (aryl), —CH(CH₃) (aryl), —CH₂— (aryl), or —C(CH₂) (aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl, or
reacting a compound having the structure: 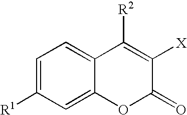
wherein X is —Br, —I, or —OTf
with any one of
(i) a compound having the structure: 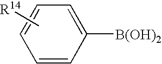
or
(ii) a compound having the structure: 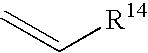
or
(iii) a compound having the structure: 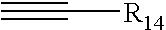
in the presence of palladium of a zero oxidation state to produce a compound having the structure: 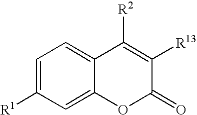
wherein R¹³ is: 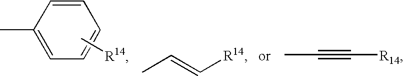
wherein R¹ is bound at carbon δ and is —H, —OH, —O-alkyl, —NH-alkyl, —N(alkyl)₂, —NH₂, aryl, heteroaryl, -alkyl-C(O) (OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl-C(O)H, -alkynyl-CH₂OH, or -alkynyl-C(O)OH; or
R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,
or R¹ is bound to carbon α and is —N(alkyl)₂, R² is —C(O)H, —CH₂OH, —C(O)OH, —C(O)CH₃, —CH(OH)CH₃, and R³ is H,
or R¹ is bound to carbon α and is —O-alkyl, R² is —CH(OH)CH₃ or —C(O)OH, and R³ is H,
or R¹ is bound to carbon β and is —O-alkyl, R² is —C(O)H, —C(O)OH, —CH₂OH, —C(O)CH₃, —CH(OH)CH₃, and R³ is H,
or R¹ is bound to carbon β and is —N(alkyl)₂, R² is —C(O)H, —C(O)OH, —CH₂OH, —C(O)CH₃, —CH(OH)CH₃, and R³ is H,
or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl-C(O)H, -alkynyl-CH₂OH, —C(O)R⁷, —CH(OH)R⁸, —R¹⁰—C(O)R⁹, —R¹⁰—CH(OH)R⁹, —C(CX₂) (aryl) where X is a halide, —C(CX₂) (alkyl) where —X is a halide, —C(CHX) (aryl) where X is a halide, —C(═NOH) (aryl), —CH(CH₃) (aryl), —CH₂— (aryl), or —C(CH₂) (aryl);
or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂) (aryl) where X is a halide, —C(CHX) (aryl) where X is a halide, —C(═NOH) (aryl), —CH(CH₃) (aryl), —CH₂— (aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl, or
reacting a compound having the structure: 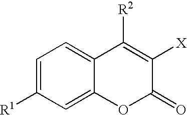
wherein X is —Br, —I, or —OTf
with any one of
(i) a compound having the structure: 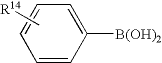
or
(ii) a compound having the structure: 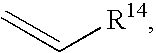
or
(iii) a compound having the structure: 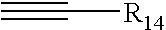
in the presence of palladium of a zero oxidation state to produce a compound having the structure: 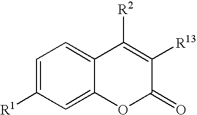
wherein R¹³ is: 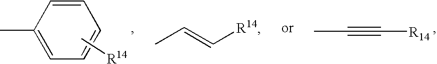
wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl-C(O)H, -alkynyl-C(O)OH, or -alkynyl-CH₂OH; or
R² and R³ together form a ring substituted with ═O,
where R⁴ is methyl, ethyl, alkenyl, alkynyl, a substituted aryl or an unsubstituted aryl, R⁵ is alkyl, alkenyl, alkynyl, substituted aryl or an unsubstituted aryl, or cycloalkyl, and R⁶ is hydrogen, methyl, a C₃-C₇ alkyl, alkenyl, alkynyl, aryl, or cycloalkyl,
or R¹ is bound to carbon α and is —N(alkyl)₂, R² is —C(O)H, —CH₂OH, —C(O)OH, —C(O)CH₃, —CH(OH)CH₃, and R³ is H,
or R¹ is bound to carbon α and is —O-alkyl, R² is —CH(OH)CH₃ or —C(O)OH, and R³ is H,
or R¹ is bound to carbon β and is —O-alkyl, R² is —C(O)H, —C(O)OH, —CH₂OH, —C(O)CH₃, —CH(OH)CH₃, and R³ is H,
or R¹ is bound to carbon β and is —N(alkyl)₂, R² is —C(O)H, —C(O)OH, —CH₂OH, —C(O)CH₃, —CH(OH)CH₃, and R³ is H,
or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl-C(O)H, -alkynyl-CH₂OH, —C(O)R⁷, —CH(OH)R⁸, —R¹⁰—C(O)R⁹, —R¹⁰—CH(OH)R⁹, —C(CX₂) (aryl) where X is a halide, —C(CX₂) (alkyl) where X is a halide, —C(CHX) (aryl) where X is a halide, —C(═NOH) (aryl), —CH(CH₃) (aryl), —CH₂— (aryl), or —C(CH₂) (aryl);
or R³ is —H or X where X is a halide, alkyl, alkenyl, alkoxy, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂) (aryl) where X is a halide, —C(CHX) (aryl)
where X is a halide, —C(═NOH) (aryl), —CH(CH₃) (aryl), —CH₂-(aryl), or —C(CH₂) (aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl; or heteroaryl, R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl, or
reacting a compound having the structure: 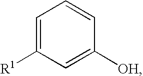
with a compound having the structure: 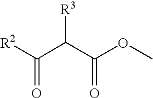
to produce a compound having the structure: 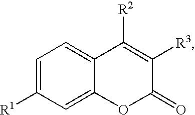
wherein R¹ is bound at carbon δ and is —H, —OH, —O-alkyl, —NH-alkyl, —N(alkyl)₂, —NH₂, aryl, heteroaryl, -alkyl-C(O) (OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵ —C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl-C(O)H, -alkynyl-CH₂OH, or -alkynyl-C(O)OH; or
R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,
or R¹ is bound to carbon α and is —N(alkyl)₂, R² is —C(O)H, —CH₂OH, —C(O)OH, —C(O)CH₃, —CH(OH)CH₃, and R³ is H,
or R¹ is bound to carbon α and is —O-alkyl, R² is —CH(OH)CH₃ or —C(O)OH, and R³ is H,
or R¹ is bound to carbon β and is —O-alkyl, R² is —C(O)H, —C(O)OH, —CH₂OH, —C(O)CH₃, —CH(OH)CH₃, and R³ is H,
or R¹ is bound to carbon β and is —N(alkyl)₂, R² is —C(O)H, —C(O)OH, —CH₂OH, —C(O)CH₃, —CH(OH)CH₃, and R³ is H,
or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl-C(O)H, -alkynyl-CH₂OH, —C(O)R⁷, —CH(OH)R⁸, —R¹⁰—C(O)R⁹, —R¹⁰—CH(OH)R⁹, —C(CX₂) (aryl) where X is a halide, —C(CX₂) (alkyl) where —X is a halide, —C(CHX) (aryl) where X is a halide, —C(═NOH) (aryl), —CH(CH₃) (aryl), —CH₂— (aryl), or —C(CH₂) (aryl);
or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂)(aryl) where X is a halide, —C(CHX)(aryl) where X is a halide, —C(═NOH)(aryl), —CH(CH₃) (aryl), —CH₂— (aryl), or —C(CH₂)(aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl, or
reacting a compound having the structure: 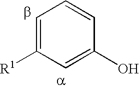
with a compound having the structure: 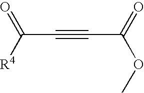
in the presence of triphenylphosphene to produce a compound having the structure: 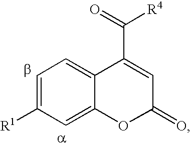
wherein R¹ is —N(CH₃)₂, —N(propyl)₂ wherein one propyl is covalently bound to carbon α and the other propyl is covalently bound to carbon β, or is >NH which is covalently bound to either carbon α or carbon β; and R⁴ is methyl or aryl, or
reacting a compound having the structure:
with a compound having the structure:
in the presence of Aluminum Chloride (AlCl3), to produce a compound having the structure:
wherein R1 is —N(CH3)2, —N(propyl)2 wherein one propyl is covalently bound to carbon α and the other propyl is covalently bound to carbon β, R1 is —N(CH3)2, or is >NH which is covalently bound to either carbon α or carbon β, and R⁴ is methyl or aryl, or
(a) reacting a compound having the structure: 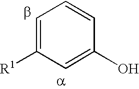
with a compound having the structure: 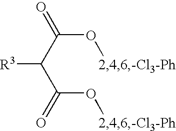
to produce a product;
(b) contacting the product of step (a) with Tf₂O (triflate) and Et₃N (triethylamine) to produce a product;
(c) contacting the product of step (b) with trimethylsilyacetylene, Pd(PPh₃)₂Cl, Copper Iodide and Et₃N to produce a product;
(d) contacting the product of step (c) with K₂CO₃ to produce a product; and
(e) contacting the product of step (d) with H₂O, HgSO₄ and H₂SO₄,
so as to produce a compound having the structure: 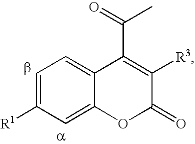
wherein R¹ is —N(propyl)₂ wherein one propyl is covalently bound to carbon α and the other propyl is covalently bound to carbon β, R¹ is —N(CH₃)₂, or is —NH which is covalently bound to either carbon α or carbon β, R³ is H, and R⁴ is methyl or aryl.

186. A composition comprising the compound of any one of claim 1 and a pharmaceutically acceptable carrier.

187. A composition comprising the compound of any one of claim 184 and a pharmaceutically acceptable carrier.

188. A method of identifying a compound not previously known to inhibit human hydroxysteroid dehydrogenase as an inhibitor of human hydroxysteroid dehydrogenase comprising:
a) transfecting a cell which does not express human hydroxysteroid dehydrogenase with a gene encoding for human hydroxysteroid dehydrogenase so that the cell expresses human hydroxysteroid dehydrogenase;
b) providing the cell in a medium;
c) contacting the cell with a reference compound that undergoes a detectable increase in fluorescence when reduced by human hydroxysteroid dehydrogenase under conditions permitting the reference compound to enter the cell;
d) detecting an increase in the fluorescence of the medium;
e) contacting the cell with the compound not previously known to inhibit human hydroxysteroid dehydrogenase under conditions permitting the compound to enter the cell; and
f) detecting a change in the fluorescence of the medium, wherein a reduced fluorescence of the medium detected in step f) compared to step d) indicates that the compound not previously known to inhibit human hydroxysteroid dehydrogenase is an inhibitor of human hydroxysteroid dehydrogenase, thereby identifying the compound as an inhibitor of human hydroxysteroid dehydrogenase, or
a) providing a human hydroxysteroid dehydrogenase in a medium;
b) contacting the human hydroxysteroid dehydrogenase with a reference compound that undergoes a detectable increase in fluorescence when reduced by human hydroxysteroid dehydrogenase under conditions permitting the reduction of the reference compound by the human hydroxysteroid dehydrogenase;
d) detecting an increase in the fluorescence of the medium;
e) contacting the human hydroxysteroid dehydrogenase with the compound not previously known to inhibit human hydroxysteroid dehydrogenase; and
f) detecting a change in the fluorescence of the medium, wherein a reduced fluorescence of the medium detected in step f) compared to step d) indicates that the compound not previously known to inhibit human hydroxysteroid dehydrogenase is an inhibitor of human hydroxysteroid dehydrogenase, thereby identifying the compound as an inhibitor of human hydroxysteroid dehydrogenase, or
a) providing a human hydroxysteroid dehydrogenase in a medium;
b) contacting the human hydroxysteroid dehydrogenase with a reference compound that undergoes a detectable decrease in fluorescence when oxidized by human hydroxysteroid dehydrogenase under conditions permitting the oxidation of the reference compound by the human hydroxysteroid dehydrogenase;
d) detecting an decrease in the fluorescence of the medium;
e) contacting the human hydroxysteroid dehydrogenase with the compound not previously known to inhibit human hydroxysteroid dehydrogenase; and
f) detecting a change in the fluorescence of the medium, wherein a reduction in the decrease of fluorescence of the medium detected in step f) compared to step d) indicates that the compound not previously known to inhibit human hydroxysteroid dehydrogenase is an inhibitor of human hydroxysteroid dehydrogenase, or
a) transfecting a cell which does not express human hydroxysteroid dehydrogenase with a gene encoding for human hydroxysteroid dehydrogenase so that the cell expresses human hydroxysteroid dehydrogenase;
b) providing the cell in a medium;
c) contacting the cell with a reference compound that undergoes a detectable decrease in fluorescence when oxidized by human hydroxysteroid dehydrogenase under conditions permitting the reference compound to enter the cell;
d) detecting a decrease in the fluorescence of the medium;
e) contacting the cell with the compound not previously known to inhibit human hydroxysteroid dehydrogenase under conditions permitting the compound to enter the cell; and
f) detecting a change in the fluorescence of the medium, wherein a reduction in the decrease of fluorescence of the medium detected in step f) compared to step d) indicates that the compound not previously known to inhibit human hydroxysteroid dehydrogenase is an inhibitor of human hydroxysteroid dehydrogenase.

189. The method of claim 188, wherein the human hydroxysteroid dehydrogenase is aldo-keto reductase 1C1, aldo-keto reductase 1C2, aldo-keto reductase 1C3, or aldo-keto reductase 1C4.

190. The method of claim 189, wherein the human hydroxysteroid dehydrogenase is a 3α-hydroxysteroid dehydrogenase, a 17β-hydroxysteroid dehydrogenase, or a 20α-hydroxysteroid dehydrogenase.

191. A method of quantitating the amount of a reductase in a sample comprising:
a) providing a sample;
b) contacting the sample with a compound that undergoes a detectable change in fluorescence when reduced by the reductase under conditions permitting reduction;
c) detecting a change in the fluorescence of the sample; and
d) quantifying the amount of reductase in the sample by comparing the fluorescence detected in step c) against a predetermined relationship between fluorescence and reductase amount. or
a) providing a sample;
b) contacting the sample with a compound that undergoes a detectable change in fluorescence when oxidized by an oxidase under conditions permitting oxidation;
c) detecting a change in the fluorescence of the sample; and
d) quantifying the amount of oxidase in the sample by comparing the fluorescence detected in step c) against a predetermined relationship between fluorescence and oxidase amount.

192. The method of claim 191, wherein the compound is the compound of claim 1.

193. The method of claim 191, wherein the compound is the compound of claim 184.

194. The method of claim 191, wherein the oxidase or reductase is a hydroxysteroid dehydrogenase.

195. A method of diagnosing a subject as suffering from a cancer of a tissue comprising:
a) obtaining a sample of the tissue which sample comprises a cell of the tissue;
b) providing the sample in a medium;
c) contacting the sample with the compound of claim 1, wherein the compound undergoes a detectable increase in fluorescence when reduced by human hydroxysteroid dehydrogenase under conditions permitting the compound to enter the cell of the tissue;
d) detecting an increase in the fluorescence of the medium; and
e) comparing the fluorescence detected in step d) with a predetermined fluorescence,
wherein fluorescence of the medium detected in step d) greater than that of the predetermined fluorescence indicates that the subject is suffering from the cancer of the tissue.

196. The method of claim 195, wherein the tissue is prostate tissue or colon tissue and the human hydroxysteroid dehydrogenase is aldo-keto reductase 1C3.

197. The method of claim 195, wherein the tissue is lung tissue the human hydroxysteroid dehydrogenase is aldo-keto reductase 1C1.

198. A compound of the structure: 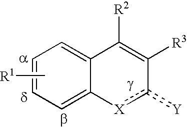
wherein
Y is O, X is O, and bond y is a single bond, or
Y is absent, X is CH and bond y is a double bond,
wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl-C(O)H, -alkynyl-C(O)OH, or -alkynyl-CH₂OH; or R² and R³ together form a ring substituted with ═O,
where R⁴ is methyl, ethyl, alkenyl, alkynyl, a substituted aryl or an unsubstituted aryl, R⁵ is alkyl, alkenyl, alkynyl, substituted aryl or an unsubstituted aryl, or cycloalkyl, and R⁶ is hydrogen, methyl, a C₃-C₇ alkyl, alkenyl, alkynyl, aryl, or cycloalkyl,
or R¹ is bound to carbon α and is —N(alkyl)₂, R² is —C(O)H, —CH₂OH, —C(O)OH, —C(O)CH₃, —CH(OH)CH₃, and R³ is H,
or R¹ is bound to carbon α and is —O-alkyl, R² is —CH(OH)CH₃ or —C(O)OH, and R³ is H,
or R¹ is bound to carbon P and is —O-alkyl, R² is —C(O)H, —C(O)OH, —CH₂OH, —C(O)CH₃, —CH(OH)CH₃, and R³ is H,
or R¹ is bound to carbon P and is —N(alkyl)₂, R² is —C(O)H, —C(O)OH, —CH₂OH, —C(O)CH₃, —CH(OH)CH₃, and R³ is H,
or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH,-alkynyl-C(O)H, -alkynyl-CH₂OH, —C(O)R⁷ —CH(OH)R⁸, —R¹⁰—C(O)R⁹, —R¹⁰—CH(OH)R⁹, —C(CX₂) (aryl) where X is a halide, —C(CX₂) (alkyl) where X is a halide, —C(CHX) (aryl) where X is a halide, —C(═NOH)(aryl), —CH(CH₃) (aryl), —CH₂— (aryl), or —C(CH₂) (aryl); or R³ is —H or X where X is a halide, alkyl, alkenyl, alkoxy, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂) (aryl) where X is a halide, —C(CHX)(aryl) where X is a halide, —C(═NOH)(aryl), —CH(CH₃) (aryl), —CH₂— (aryl), or —C(CH₂) (aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸is hydroxyl, alkyl, cycloalkyl, alkenyl, alkynyl, aryl, or heteroaryl; R⁹is alkyl, cycloalkyl, alkenyl, alkynyl, aryl; or heteroaryl, R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl,
wherein when R¹ is —N(CH₃)₂ and is bound at carbon δ and R³ is —C(O)CH₃, -alkynyl-C(O)CH₃, -alkynyl-C(O)CH₃, or —CH(OH) (CH₃), or R¹ is —O-alkyl and is bound at carbon δ and R³ is —C(O)H, then R² is OH, a C₂-C₇ alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁴, —CH(OH)R⁴, —R⁵—C(O)R⁴, or —R⁵—CH(OH)R⁴, Y is O, X is O and bond γ is a single bond, and
wherein when R¹ is —N(propyl)₂ wherein one propyl is covalently bound to carbon α and the other propyl is covalently bound to carbon β, and R² is —C(O)CH₃ or —C(O)OH, and R³ is —H, then Y is absent, X is CH and bond γ is a double bond,
wherein when R¹ is —N(propyl)₂ wherein one propyl is covalently bound to carbon α and the other propyl is covalently bound to carbon β, and R³ is -methylcarbonylphenyl, methylhydroxyphenyl, —C≡C—C(O)CH₃, or —C≡C—CH(OH)—CH₃, and R² is —H, then Y is absent, X is CH and bond γ is a double bond,
wherein when R¹ is —OCH₃ and is bound to carbon δ, and R³ is methylcarbonylphenyl, methylhydroxyphenyl, —C≡C—C(O)CH₃, or —C≡C—CH(OH)—CH₃, and R² is —H, then Y is absent, X is CH and bond γ is a double bond,
or a salt or stereoisomer thereof.

199. A process for preparing the compound of claim 1 comprising:
reacting a compound having the structure: 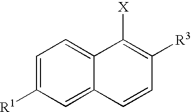
wherein X is —Br, —I, or —OTf
with any one of
(i) a compound having the structure: 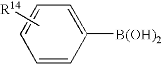
or
(ii) a compound having the structure: 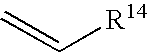
or
(iii) a compound having the structure: 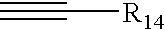
in the presence of palladium of a zero oxidation state to produce a compound having the structure: 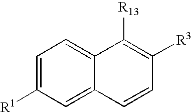
wherein R¹³ is: 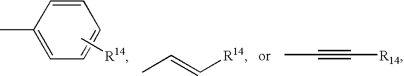
wherein R¹⁴ is any of R² or R³,
wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH(OH)R⁴, -aryl-C(O)H, -aryl-CH₂OH, -aryl-C(O)OH, -alkynyl-C(O)H, -alkynyl-CH₂OH, or -alkynyl-C(O)OH; or R² and R³ together form a ring substituted with ═O;
where R⁴ is methyl, ethyl, alkenyl, alkynyl, substituted aryl or unsubstituted aryl, R⁵ is alkyl, alkenyl, alkynyl, substituted aryl unsubstituted aryl, or cycloalkyl; and R⁶ is hydrogen, methyl, a C₃-C₇ alkyl, alkenyl, alkynyl, aryl, or cycloalkyl,
or R¹ is bound to carbon α and is —N(alkyl)₂, R² is —C(O)H, —CH₂OH, —C(O)OH, —C(O)CH₃, —CH(OH)CH₃, and R³ is H,
or R¹ is bound to carbon α and is —O-alkyl, R² is —CH(OH)CH₃ or —C(O) OH, and R³ is H,
or R¹ is bound to carbon β and is —O-alkyl, R² is —C(O)H, —C(O)OH, —CH₂OH, —C(O)CH₃, —CH(OH)CH₃, and R³ is H,
or R¹ is bound to carbon β and is —N(alkyl)₂, R² is —C(O)H, —C(O)OH, —CH₂OH, —C(O)CH₃, —CH(OH)CH₃, and R³ is H,
or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl-C(O)H, -alkynyl-CH₂OH, —C O)R⁷, —CH(OH)R⁸, —R¹⁰—C(O)R⁹, —R¹⁰—CH(OH)R⁹, —C(CX₂) (aryl) where X is a halide, —C(CX₂) (alkyl) where —X is a halide, —C(CHX) (aryl) where X is a halide, —C(═NOH) (aryl), —CH(CH₃) (aryl), —CH₂— (aryl), or —C(CH₂) (aryl); or R³ is —H, or X where X is a halide, alkyl, alkenyl, alkoxy, or aryl or cycloalkyl, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂) (aryl) where X is a halide, —C(CHX) (aryl) where X is a halide, —C(═NOH) (aryl), —CH (CH₃) (aryl), —CH₂— (aryl), or —C(CH₂) (aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl, or heteroaryl; R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl.

200. A process for preparing the compound of claim 40 comprising:
reacting a compound having the structure: 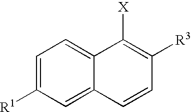
wherein X is —Br, —I, or —OTf
with any one of
(i) a compound having the structure: 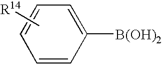
or
(ii) a compound having the structure: 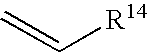
or
(iii) a compound having the structure: 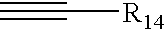
in the presence of palladium of a zero oxidation state to produce a compound having the structure: 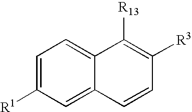
wherein R¹³ is: 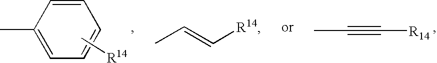
wherein R¹⁴ is any of R² or R³,
wherein R¹ is bound at carbon δ and is —H, —OH, —O-alkyl, —NH-alkyl, —N(alkyl)₂, —NH₂, aryl, heteroaryl, -alkyl-C(O)(OH), -alkyl-OH, or R¹ is bound at carbon δ and is >NH which is covalently bound to carbon α or to carbon β and is unsubstituted or substituted at the nitrogen atom and/or at a carbon atom; R² is H, OH, a C₂-C₇ alkyl, alkenyl, alkynyl, aryl, cycloalkyl, —O-alkyl, —O-alkenyl, —O-alkynyl, —O-aryl which aryl may be substituted or unsubstituted, —O-cycloalkyl, —NH-alkyl, —N(alkyl)₂, halide, —C(O)R⁴, —CH(OH)R⁴, —R⁵—C(O)R⁴, or —R⁵—CH(OH)R⁴; and R³ is H, alkyl, alkenyl, alkynyl, aryl, cycloalkyl, —O-alkyl, —O-alkenyl, —O-alkynyl, —O-aryl, —O-cycloalkyl, —NH-alkyl, —N(alkyl)₂, halide, —C(O)R⁶, —CH(OH)R⁴, —R⁵—C(O)R⁴, —R⁵—CH (OH)R⁴, -aryl-C(O)H, -aryl-CH₂OH, -aryl-C(O)OH, -alkynyl-C(O)H, -alkynyl-C(O)OH, or -alkynyl-CH₂OH; or R² and R³ together form a ring substituted with ═O,
where R⁴ is methyl, ethyl, alkenyl, alkynyl, a substituted aryl or an unsubstituted aryl, R⁵ is alkyl, alkenyl, alkynyl, substituted aryl or an unsubstituted aryl, or cycloalkyl, and R⁶ is hydrogen, methyl, a C₃-C₇ alkyl, alkenyl, alkynyl, aryl, or cycloalkyl,
or R¹ is bound to carbon α and is —N(alkyl)₂, R² is —C(O)H, —CH₂OH, —C(O)OH, —C(O)CH₃, —CH(OH)CH₃, and R³ is H,
or R¹ is bound to carbon α and is —O-alkyl, R² is —CH(OH)CH₃ or —C(O)OH, and R³ is H,
or R¹ is bound to carbon β and is —O-alkyl, R² is —C(O)H, —C(O)OH, —CH₂OH, —C(O)CH₃, —CH(OH)CH₃, and R³ is H,
or R¹ is bound to carbon β and is —N(alkyl)₂, R² is —C(O)H, —C(O)OH, —CH₂OH, —C(O)CH₃, —CH(OH)CH₃, and R³ is H,
or R¹ is bound to carbon δ and is —N< which is covalently bound to both carbon α and carbon β and either R² is —H and R³ is —C(O)H, —CH₂OH, -aryl-C(O)H, -aryl-CH₂OH, -aryl-C(O)OH, -alkynyl-C(O)H, -alkynyl-CH₂OH, —C(O)R⁷, —CH(OH)R⁸, —R¹⁰—C(O)R⁹, —R¹⁰—CH(OH)R⁹, —C(CX₂) (aryl) where X is a halide, —C(CX₂) (alkyl) where X is a halide, —C(CHX) (aryl) where X is a halide, —C(═NOH)(aryl), —CH(CH₃) (aryl), —CH₂— (aryl), or —C(CH₂) (aryl); or R³ is —H or X where X is a halide, alkyl, alkenyl, alkoxy, and R² is —C(O)H, —C(O)R¹¹, —CH(OH)CH₃, —CH(OH)R⁷, —R¹⁰—C(O)R⁹, —R¹⁰—CH(OH)R⁹, —C(CX₂) (aryl) where X is a halide, —C(CHX) (aryl) where X is a halide, —C(═NOH)(aryl), —CH(CH₃) (aryl), —CH₂— (aryl), or —C(CH₂) (aryl); or R² and R³ together form a ring substituted with ═O or —OH; or R² is —C(O)CH₃ or —CH(OH)CH₃, and R³ is aryl;
where R⁷ is cycloalkyl, C₂-C₇ alkyl, alkenyl, alkynyl, aryl, or heteroaryl; R⁸ is hydroxyl, alkyl, cycloalkyl, alkenyl, alkynyl, aryl, or heteroaryl; R⁹ is alkyl, cycloalkyl, alkenyl, alkynyl, aryl; or heteroaryl, R¹⁰ is alkynyl, aryl, or heteroaryl; and R¹¹ is methyl, isopropyl, hydroxyl, alkenyl, alkynyl, cycloalkyl, —O-alkyl, aryl, or heteroaryl.

201. A method of identifying a compound not previously known to inhibit human hydroxysteroid dehydrogenase as an inhibitor of human hydroxysteroid dehydrogenase comprising:
a) transfecting a cell which does not express human hydroxysteroid dehydrogenase with a gene encoding for human hydroxysteroid dehydrogenase so that the cell expresses human hydroxysteroid dehydrogenase;
b) providing the cell in a medium;
c) contacting the cell with a reference compound that undergoes a detectable increase in fluorescence when reduced by human hydroxysteroid dehydrogenase under conditions permitting the reference compound to enter the cell;
d) detecting an increase in the fluorescence of the medium;
e) contacting the cell with the compound not previously known to inhibit human hydroxysteroid dehydrogenase under conditions permitting the compound to enter the cell; and
f) detecting a change in the fluorescence of the medium, wherein a reduced fluorescence of the medium detected in step f) compared to step d) indicates that the compound not previously known to inhibit human hydroxysteroid dehydrogenase is an inhibitor of human hydroxysteroid dehydrogenase, thereby identifying the compound as an inhibitor of human hydroxysteroid dehydrogenase.

202. The method of diagnosing a subject as suffering from a cancer of a tissue comprising:
a) obtaining a sample of the tissue which sample comprises a cell of the tissue;
b) providing the sample in a medium;
c) contacting the sample with a compound that undergoes a detectable increase in fluorescence when reduced by human hydroxysteroid dehydrogenase under conditions permitting the compound to enter the cell of the tissue;
d) detecting an increase in the fluorescence of the medium; and
e) comparing the fluorescence detected in step d) with a predetermined fluorescence, wherein fluorescence of the medium detected in step d) greater than that of the predetermined fluorescence indicates that the subject is suffering from the cancer of the tissue.

203. A method of diagnosing a subject as suffering from a cancer of a tissue comprising:
a) obtaining a sample of the tissue which sample comprises a cell of the tissue;
b) obtaining a cellular fraction from the sample;
c) contacting the cellular fraction with a compound that undergoes a detectable increase in fluorescence when reduced by human hydroxysteroid dehydrogenase;
d) detecting an increase in the fluorescence of the cellular fraction; and
e) comparing the fluorescence detected in step d) with a predetermined fluorescence,
wherein fluorescence of the cellular fraction detected in step d) greater than that of the predetermined fluorescence indicates that the subject is suffering from the cancer of the tissue.

204. A method of treating a cancer in a subject comprising administering to the cancer in the subject an amount of the compound of claim 10 effective to treat the cancer.

205. A method of identifying a compound not previously known to inhibit human hydroxysteroid dehydrogenase as an inhibitor of human hydroxysteroid dehydrogenase comprising:
a) providing a human hydroxysteroid dehydrogenase in a medium;
b) contacting the human hydroxysteroid dehydrogenase with a reference compound that undergoes a detectable increase in fluorescence when reduced by human hydroxysteroid dehydrogenase under conditions permitting the reduction of the reference compound by the human hydroxysteroid dehydrogenase;
d) detecting an increase in the fluorescence of the medium;
e) contacting the human hydroxysteroid dehydrogenase with the compound not previously known to inhibit human hydroxysteroid dehydrogenase; and
f) detecting a change in the fluorescence of the medium, wherein a reduced fluorescence of the medium detected in step f) compared to step d) indicates that the compound not previously known to inhibit human hydroxysteroid dehydrogenase is an inhibitor of human hydroxysteroid dehydrogenase, thereby identifying the compound as an inhibitor of human hydroxysteroid dehydrogenase.

206. A method of identifying a compound not previously known to inhibit human hydroxysteroid dehydrogenase as an inhibitor of human hydroxysteroid dehydrogenase comprising:
a) providing a human hydroxysteroid dehydrogenase in a medium;
b) contacting the human hydroxysteroid dehydrogenase with a reference compound that undergoes a detectable decrease in fluorescence when oxidized by human hydroxysteroid dehydrogenase under conditions permitting the oxidation of the reference compound by the human hydroxysteroid dehydrogenase;
d) detecting an decrease in the fluorescence of the medium;
e) contacting the human hydroxysteroid dehydrogenase with the compound not previously known to inhibit human hydroxysteroid dehydrogenase; and
f) detecting a change in the fluorescence of the medium, wherein a reduction in the decrease of fluorescence of the medium detected in step f) compared to step d) indicates that the compound not previously known to inhibit human hydroxysteroid dehydrogenase is an inhibitor of human hydroxysteroid dehydrogenase.

207. A method of identifying a compound not previously known to inhibit human hydroxysteroid dehydrogenase as an inhibitor of human hydroxysteroid dehydrogenase comprising:
a) transfecting a cell which does not express human hydroxysteroid dehydrogenase with a gene encoding for human hydroxysteroid dehydrogenase so that the cell expresses human hydroxysteroid dehydrogenase;
b) providing the cell in a medium;
c) contacting the cell with a reference compound that undergoes a detectable decrease in fluorescence when oxidized by human hydroxysteroid dehydrogenase under conditions permitting the reference compound to enter the cell;
d) detecting a decrease in the fluorescence of the medium;
e) contacting the cell with the compound not previously known to inhibit human hydroxysteroid dehydrogenase under conditions permitting the compound to enter the cell; and
f) detecting a change in the fluorescence of the medium, wherein a reduction in the decrease of fluorescence of the medium detected in step f) compared to step d) indicates that the compound not previously known to inhibit human hydroxysteroid dehydrogenase is an inhibitor of human hydroxysteroid dehydrogenase.

208. A method of quantitating the amount of a reductase in a sample comprising:
a) providing a sample;
b) contacting the sample with a compound that undergoes a detectable change in fluorescence when reduced by the reductase under conditions permitting reduction;
c) detecting a change in the fluorescence of the sample; and
d) quantifying the amount of reductase in the sample by comparing the fluorescence detected in step c) against a predetermined relationship between fluorescence and reductase amount.

209. A method of quantitating the amount of an oxidase in a sample comprising:
a) providing a sample;
b) contacting the sample with a compound that undergoes a detectable change in fluorescence when oxidized by an oxidase under conditions permitting oxidation;
c) detecting a change in the fluorescence of the sample; and
d) quantifying the amount of oxidase in the sample by comparing the fluorescence detected in step c) against a predetermined relationship between fluorescence and oxidase amount.
